SEQUENCE LISTING (29 sequences)

SEQ ID NO: 1 - DNA, 20 bp, Artificial sequence
  single strand DNA oligonucleotide
tgtcatcacccaattttcca

SEQ ID NO: 2 - DNA, 21 bp, Artificial sequence
  single strand DNA oligonucleotide
ttccaggaacttggtttcctt

SEQ ID NO: 3 - DNA, 20 bp, Artificial sequence
  single strand DNA oligonucleotide
tgaaagggcataccaaaagg

SEQ ID NO: 4 - DNA, 20 bp, Artificial sequence
  single strand DNA oligonucleotide
ggctacgccaagaactctga

SEQ ID NO: 5 - DNA, 26 bp, Artificial sequence
  single strand DNA oligonucleotide
gttctagatgtagacaaaagagtgga

SEQ ID NO: 6 - DNA, 27 bp, Artificial sequence
  single strand DNA oligonucleotide
acatctagatatcatgctagtgtcctt

SEQ ID NO: 7 - DNA, 24 bp, Artificial sequence
  single strand DNA oligonucleotide
gctgatttggatatctatggtttc

SEQ ID NO: 8 - DNA, 28 bp, Artificial sequence
  single strand DNA oligonucleotide
aactcgagttgtatttggatgatttgca

SEQ ID NO: 9 - DNA, 25 bp, Artificial sequence
  single strand DNA oligonucleotide
ttgtgtttgccgctccagtggatat

SEQ ID NO: 10 - DNA, 24 bp, Artificial sequence
  single strand DNA oligonucleotide
gcgccttccattgaagccaagtat

SEQ ID NO: 11 - DNA, 22 bp, Artificial sequence
  single strand DNA oligonucleotide
ggggaatttgggcttgttgagt

SEQ ID NO: 12 - DNA, 22 bp, Artificial sequence
  single strand DNA oligonucleotide
cctttgattcaggggcgatacc

SEQ ID NO: 13 - DNA, 20 bp, Artificial sequence
  single strand DNA oligonucleotide
gatcgccaaatccttagcaa

SEQ ID NO: 14 - DNA, 2388 bp, Lycopersicon esculentum
ggaagaacccttcactttgctcatacccacctcatcaaaatccacctaaatgacttctctcactttgctcaatcatctatatagcctaagaattgtcaagattccatttttatagttgttggtgtctgtaatcttggaatttgaacaatttaaagtgaaaagattcaagtttttagaattttctctgcttttgcagttgcagaggtaaagagttgtcaagattccatttttgtagtttattgtgtttgtaatcttgggtttccagcaatttaaagaaaaaaagattcaatctttttaatttatcagtattttggcagctgcagaagtaaagaattggatagcttgaaacccacaaggcaaaagttctagtcttgtttggttaactttccagggagcccaaaattttgtgaaataagagaaatgtgtaccttgagttttatgtatcctaattcacttcttgatggtacctgcaagactgtagctttgggtgatagcaaaccaagatacaataaacagagaagttcttgttttgaccctttgataattggaaattgtactgatcagcagcagctttgtggcttgagttggggggtggacaaggctaagggaagaagagggggtactgtttccaatttgaaagcagttgtagatgtagacaaaagagtggagagctatggcagtagtgatgtagaaggaaatgagagtggcagctatgatgccattgttataggttcaggaataggtggattggtggcagcgacgcacctggcggttaagggagctaaggttttagttctggagaagtatgttattcctggtggaagctctggcttttacgagagggatggttataagtttgatgttggttcatcagtgatgtttggattcagtgataagggaaacctcaatttaattactcaagcattggcagcagtaggacgtaaattagaagttatacctgacccaacaactgtacatttccacctgccaaatgacctttctgttcgtatacaccgagagtatgatgacttcattgaagagcttgtgagtaaatttccacatgaaaaggaagggattatcaaattttacagtgaatgctggaagatctttaattctctgaattcattggaactgaagtctttggaggaacccatctacctttttggccagttctttaagaagccccttgaatgcttgactcttgcctactatttgccccagaatgctggtagcatcgctcggaagtatataagagatcctgggttgctgtcttttatagatgcagagtgctttatcgtgagtacagttaatgcattacaaacaccaatgatcaatgcaagcatggttctatgtgacagacattttggcggaatcaactaccccgttggtggagttggcgagatcgccaaatccttagcaaaaggcttggatgatcacggaagtcagatactttatagggcaaatgttacaagtatcattttggacaatggcaaagctgtgggagtgaagctttctgacgggaggaagttttatgctaaaaccatagtatcgaatgctaccagatgggatacttttggaaagcttttaaaagctgagaatctgccaaaagaagaagaaaatttccagaaagcttatgtaaaagcaccttcttttctttctattcatatgggagttaaagcagatgtactcccaccagacacagattgtcaccattttgtcctcgaggatgattggacaaatttggagaaaccatatggaagtatattcttgagtattccaacagttcttgattcctcattggccccagaaggacaccatattcttcacatttttacaacatcgagcattgaagattgggagggactctctccgaaagactatgaagcgaagaaagaggttgttgctgaaaggattataagcagacttgaaaaaacactcttcccagggcttaagtcatctattctctttaaggaggtgggaactccaaagacccacagacgataccttgctcgtgatagtggtacctatggaccaatgccacgcggaacacctaagggactcctgggaatgcctttcaataccactgctatagatggtctatattgtgttggcgatagttgcttcccaggacaaggtgttatagctgtagccttttcaggagtaatgtgcgctcatcgtgttgcagctgacttagggtttgaaaaaaaatcagatgtgctggacagtgctcttcttagactacttggttggttaaggacactagcatgatatctacttgttaagctaaaaagaaacatttgacacaaaaaaagaaagttcattatgtatttactttttctttatatcaataaactggaaaatgaggttccaattgaggttcctccagta

SEQ ID NO: 15 - protein, 615 aa, Lycopersicon esculentum
  misc_feature (338)..(338): Any amino acid
MCTLSFMYPNSLLDGTCKTVALGDSKPRYNKQRSSCFDPLIIGNCTDQQQLCGLSWGVDKAKGRRGGTVSNLKAVVDVDKRVESYGSSDVEGNESGSYDAIVIGSGIGGLVAATHLAVKGAKVLVLEKYVIPGGSSGFYERDGYKFDVGSSVMFGFSDKGNLNLITQALAAVGRKLEVIPDPTTVHFHLPNDLSVRIHREYDDFIEELVSKFPHEKEGIIKFYSECWKIFNSLNSLELKSLEEPIYLFGQFFKKPLECLTLAYYLPQNAGSIARKYIRDPGLLSFIDAECFIVSTVNALQTPMINASMVLCDRHFGGINYPVGGVGEIAKSLAKGLDXHGSQILYRANVTSIILDNGKAVGVKLSDGRKFYAKTIVSNATRWDTFGKLLKAENLPKEEENFQKAYVKAPSFLSIHMGVKADVLPPDTDCHHFVLEDDWTNLEKPYGSIFLSIPTVLDSSLAPEGHHILHIFTTSSIEDWEGLSPKDYEAKKEVVAERIISRLEKTLFPGLKSSILFKEVGTPKTHRRYLARDSGTYGPMPRGTPKGLLGMPFNTTAIDGLYCVGDSCFPGQGVIAVAFSGVMCAHRVAADLGFEKKSDVLDSALLRLLGWLRTLA

SEQ ID NO: 16 - DNA, 7998 bp, Lycopersicon esculentum
  misc_feature (4713)..(4713): Any nucleotide
tctagagtcctgaatcaatcaaaataacattttttacccaaaatcaccaaagaaaatccaaatcaactcaacccaacaccaaaattcaatcaagttcatcctcatatcttcctcagtatacacaaatttatacagagacaatcagattctactcagaaccgcattcttacttcaaatcccaatacaggttgaacagattagttaatcctcagcagcaaatctaatactaacacatatggcccattacacctaaaattttaacctatgatctaacataatcttgaacacccttttgccacttcactataacaaagggattcaacacttcatacatataaaaaaactaatcttttctctattcccacaatatagttttcaaaaacaaattagtcgagatacacgcaaattaatcaccagaaatgtacaaaaatgtaaagagggagcaaaagggttacctgaaattggatttggggaaacaaaaaaatgaatcttttgggtgtaaagagatcgcaaataggcaaaagacagttaattgcgtatagccatgtggatccaaattgtttggcgcttcttttgtaagcacaaagctaacccaatatgggataatataatttttattcctgtccaaacaaaaaaaattcttttaaattttgatcttagattaaattaaaaaaacatgagaaaaaaaaaagaaaagaaaaagaatatgcttttcactaggtgtttggaattttgcttgtagcttcgtttattatttataatacaatatgaaatcattataatattggatcggtaattattgttatagacgaacaaaactagtttatatgaatcacaaataatattacgggcctaacatgatattgaattagccgtttgaccaccaatgctccgaatgaggggtaggattctttatttttttctcgaaaagttatttatgtgaaatatattcctaacacattgaaactatttcgaaatctaaccaaatatgttagtggagcttttgacattactccgcgtccgtggataagattgttaggagaaagaatgttgaatataaagtcgaatcaatctgaattcacctcctcacgtaagattactatagaagaaattttttcctattaaatattatgtaaagtcaaaacattttttgcatattcacaatccaatttctcccacctataggagaaacctaaaagataactacccattaaccaaagccagcaagcaccaaaaatttggccagcaaaaaaataccatattgtctcattccactttgaatcacaaaagtagaatcatgccaatccacttctctttccatttcaagcactacaaacaagggaagaacccttcactttgctcatacccacctcatcaaaatccacctaaatacttctctcactttgctcaatcatctatatagcctaagaattgtcaagattccatttttatagttgttggtgtctgtaatcttggaatttgaacaatttaaagtgaaaagattcaagtttttagaattttctctgcttttgcagttgcagaggtaaagagttgtcaagattccatttttgtagtttattgtgtttgtaatcttgggtttccagcaatttaaagaaaaaaagattcaatctttttaatttatcagtattttggcagctgcagaagtaaagaattggatagcttgaaacccacaaggcaaaagttctagtcttgtttggttaactttccagggagcccaaaattttgtgaaataagagaaatgtgtaccttgagttttatgtatcctaattcacttcttgatggtacctgcaagactgtagctttgggtgatagcaaaccaagatacaataaacagagaagttcttgttttgaccctttgataattggaaattgtactgatcagcagcagctttgtggcttgagttggggggtggacaaggctaagggaagaagagggggtactgtttccaatttgaaagcagttgtagatgtagacaaaagagtggagagctatggcagtagtgatgtagaaggaaatgagagtggcagctatgatgccattgttataggttcaggaataggtggattggtggcagcgacgcagctggcggttaagggagctaaggttttagttctggagaagtatgttattcctggtggaagctctggcttttacgagagggatggttataagtttgatgttggttcatcagtgatgtttggattcagtgataaggttagttgtctaaatacttgctttctgttatttaggacataacacagaatacttgctttatttagacagaaagcaagtatttagacaagtacactaaatatcacgaatcgatcaacaagtacacaacacacaccgctccatagtctgaaagtgtggaagtgattatgttttcattcaagaatgcatccaaatattacgtataatgactacatctttaacaattttttttaatcaagtatggttttcattgataataacaaggccaacttggtcatatacaagagatataccaaaaaggtcattaacatagtttatactggccatggtgtatgctttttactattgtatatggtctgcagtgcttccacgtgattggactactgttgttcctggttagatttaaactaatgtctcacatcttttgacagggaaacctcaatttaattactcaagcattggcagcagtaggacgtaaattagaagttatacctgacccaacaactgtacatttccacctgccaaatgacctttctgttcgtatacaccgagagtatgatgacttcattgaagagcttgtgagtaaatttccacatgaaaaggaagggattatcaaattttacagtgaatgctggaaggtttgtcttcattaaggtgtctgcaggtttggcttaaattttggactccttgatttagactcaaatcatatgatgaatatggatgcacagatctttaattctctgaattcattggaactgaagtctttggaggaacccatctacctttttggccagttctttaagaagccccttgaatgcttgactcttggtaagtttttccttgctttaacttcaaatatagtattccttgaattcgcacatatccattggttggactaattcagtaataagtccccaatcatggagtcactttttatccccaagggcttagttcaagtagcaaagtttgtgggacttgtgactttggtcaccggtttgagccctgtggcatgcgaacaaagctagttatttaagtgaagaatggtaaaggggaggggccattatccccgagttttgagcactattgatggtcctgagtgtttgccctcggtcatcaaaaaaatttaaggagtcatctttcacgctgatgtgtgcagcgcgcgacgtgcttaattatcctaccgtagaatcttaatttatgccatcattattcattacagcctactatttgccccagaatgctggtagcatcgctcggaagtatataagagatcctgggttgctgtcttttatagatgcagaggtgaggcgaataatctgtgttacttattatcagctccaatgatttctttacctcttaaagaattcaactccattgtctcttttgcagtgctttatcgtgagtacagttaatgcattacaaacaccaatgatcaatgcaagcatggtaattctgcatattaaccttaggccgtgttgtgtttcgtgatattcagttccacttcctcgtgagttttctttctgcaggttctatgtgacagacattttggcggaatcaactaccccgttggtggagttggcgagatcgccaaatccttagcaaaaggcttggatgatcacggaagtcagatactttatagggcaaatgttacaagtatcattttggacaatggcaaagctgtgagttttctgtcgtaagactatttaacttttcttatgattgattattcccatcataaaatgcaaaagctgtagtctggtgtttgtgttagaaagttgtagccgacactcttttgatgttcaaggactcatgcttcacacactcataactgtggtaaaatcttcttcccatgtgatacgcctttggtgtccctaaatgcatgccgttttaaaatttaaggttacattagcatgtcagagtgttaaaacattataagaaaagaattggctagtaagtatatagaaaaagaagaaaagaatgaggagaacgaagagggaagaacaaattacttctgttaaaatgtcatgcatttgttatagttgattgtaattgatatagtgtactattttgcttatcacaacattgtgtagattcacatcccctttaattgtatttcagttacatccctgaagttattctactgatgtagggtctgtacttttactgttctacatacatgaataaattgtacctaacaatataatgacattgtatgttgccatgtcataatggtgtcttgtgtagcatcttcaatgttgtctttgccagtatctgcgcaagttcattttctctaattcttctttttctttaggtgggagtgaagctttctgacgggaggaagttttatgctaaaaccatagtatcgaatgctaccagatgggatacttttggttagtttacaaggaatccagcaaaacttatatgttttcttaagattctttcgtcttagggccagtactgtggtattcttgcacatattntgattctctttaaatctcgtcatctcatgtcaagtgtcttacatctgtaggaaagcttttaaaagctgagaatctgccaaaagaagaagaaaatttccagaaagcttatgtaaaagcaccttcttttctttctattcatatgggagttaaagcagatgtactcccaccagacacagattgtcaccattttgtcctcgaggtactacggcagttcaattgatttgctatttattttttcatacttgatgttcccattcatgattccttgattttacaggatgattggacaaatttggagaaaccatatggaagtatattcttgagtattccaacagttcttgattcctcattggccccagaaggacaccatattcttcacatttttacaacatcgagcattgaagattgggaggtaaattcagtacactccttgagtgttgttggtagtaacctcttccacatctatgtctctttttcctttttatggaaattaaagtatggccctttatccttactagggactctctccgaaagactatgaagcgaagaaagaggttgttgctgaaaggattataagcagacttgaaaaaacactcttcccagggcttaagtcatctattctctttaaggaggttaagttcgtgattttatgaactcaatagttgttcataatgagcaatattatctgtcttcaatagcaaatccacatgctcttatgcttgctgaaatagttttggccgtggagttacaccatctatgtttacaattgaattcttgtaggtgggaactccaaagacccacagacgataccttgctcgtgatagtggtacctatggaccaatgccacgcggaacacctaagggactcctgggaatgcctttcaataccactgtgagttaatcatcctttacgtatgtagttgcttttattgktctgctggtacaaacagaataaactgttccgtaatgtttctgagcttacaggctatagatggtctatattgtgttggcgatagttgcttcccaggacaaggtgttatagctgtagccttttcaggagtaatgtgcgctcatcgtgttgcagctgacttaggtaaacatgatagtccaaatagttcattttctgagcttgaaaatcctaataacatttgtgcaatatctatttacacatcataagctcgaccaaatatttataatgtggccttcaaaaatgaaaatgaatgagtatttacaacgtcgagcatcaatagccccttaaatttgctagtaaatttcatttagacactctaactataacatattacaattgagcacgtgaacacatgataaagcgcctgttatataagataagtcaaccacacaaaatatgtgacctcctccttcaactatacacatcagctttaattggaacaagggagaaggtttatggcttartcaaagggttgtcsatgtggttccggcctttaattttgaaagttccattattaaggtaacctgtctgtttggtttgacatcttctcttttttctgtcacagggtttgaaaaaaaatcagatgtgctggacagtgctcttcttagactacttggttggttaaggacactagcatgatatctacttgttaagctaaaaagaaacatttgacacaaaaaaagaaagttcattatgtatttactttttctttatatcaataaactggaaaatgaggttccaattgaggttcctccagtatagaaatcacagattttaattttgcaaattctgctaattgtcttctttttcttttctgcaactttctgccaaaggatgtcaatctcttagttatcatgaatttgatctccacctttgcattacatataatgcatccctttatgctcattaaacaagagacattgactttacatatgtatacaagttttggtgttctataatcaaaagaagatattgaaactttaattgccataactaagtgttttacatataagttacatatattaagaatatatcaaaccaaaaacaagactgaagtatagattgctttgtgcagtgcttaaaattcaatgttaagctgaaaagaaacagtttttagctaagttctcttgttcacaaacagattcataaattttaaccaaacagaggtgacttcaaagacatgtatattgtacctgttgtgcaaacatcagaagattgttctcaaccttctcaagaatagcatcagatggtgcaaacaataaaaacacaggaaactatcatttatattcccaatcctcgcgaatagtggtgagggcaggattttcactaatgaggtctaaaatataaagaagtaaaaacacacgaagaagttatagggagattcattgtctacaactgaaccttgtttatactgtgtaatttcagtccatctggtaacaaccagatccatagttatgaaacaaaaagaaaattcccagatatatgttgtatgttacattgatgcagaaaatgacttattaaaacatagctactctagtcccctactgaggctgaatcaataacaatacaccmttcagtacagttgcaactattatggttcattaatatcaaaatgttaccatcatgtcagcaagttaccttagcaatcaagtttaaaaatctcaagaagatccctaaaaatgaggtactcacaagaaatggtgagacaattaactaatgaactctggattatcctgcagggtctggctcgtcatttcacatcgatccaaattctacctctgcttggaatgcggtaaccaaaggatccaagaaatggatattatttcccccggatgtggtgccaccaggggttcatccaagccctgacggtgcanaantagcaagtcctgtttcaatcatagaatggttcatgaacttttacaacgcaaccaagaattggaaaaagagacctatcgaatgtatctgcaaggcgggtgaagttatttttgtacctaatggatggtggcatttggtcatcaattnanaggattcaattgccattacacagaacttcgttagcaggtatgctacttttgtctcctatatttctgtcacttgataaaatttgatattgttttnatgcttgctgacaaataattatatgtactagtttccttaatagcatgtaatggttctgttttataaggtggacactagatataatgcaaaagttcactattggtttagtagctgtaatttaatatcacatatactctcaggttaagctcattaactagaaaaacgaaccctcaactcacgaatgtttgcttgtttcga

SEQ ID NO: 17 - DNA, 7650 bp, Lycopersicon esculentum
  misc_feature: CrtISO genomic DNA sequence, gene t3183
tctagagtcctgaatcaatcaaaataacattttttacccaaaatcaccaaagaaaatccaaatcaactcaacccaacaccaaaattcaatcaagttcatcctcatatcttcctcagtatacacaaatttatacagagacaatcagattctactcagaaccgcattcttacttcaaatcccaatacaggttgaacagattagttaatcctcagcagcaaatctaatactaacacatatggcccattacacctaaaattttaacctatgatctaacataatcttgaacacccttttgccacttcactataacaaagggattcaacacttcatacatataaaaaaactaatcttttctctattcccacaatatagttttcaaaaacaaattagtcgagatacacgcaaattaatcaccagaaatgtacaaaaatgtaaagagggagcaaaagggttacctgaaattggatttggggaaacaaaaaaatgaatcttttgggtgtaaagagatcgcaaataggcaaaagacagttaattgcgtatagccatgtggatccaaattgtttggcgcttcttttgtaagcacaaagctaacccaatatgggataatataatttttattcctgtccaaacaaaaaaaattcttttaaattttgatcttagattaaattaaaaaaacatgagaaaaaaaaaagaaaagaaaaagaatatgcttttcactaggtgtttggaattttgcttgtagcttcgtttattatttataatacaatatgaaatcattataatattggatcggtaattattgttatagacgaacaaaactagtttatatgaatcacaaataatattacgggcctaacatgatattgaattagccgtttgaccaccaatgctccgaatgaggggtaggattctttatttttttctcgaaaagttatttatgtgaaatatattcctaacacattgaaactatttcgaaatctaaccaaatatgttagtggagcttttgacattactccgcgtccgtggataagattgttaggagaaagaatgttgaatataaagtcgaatcaatctgaattcacctcctcacgtttccatttttatagttgttggtgtctgtaatcttggaatttgaacaatttaaagtgaaaagattcaagtttttagaattttctctgcttttgcagttgcagaggtaaagagttgtcaagattccatttttgtagtttattgtgtttgtaatcttgggtttccagcaatttaaagaaaaaaagattcaatctttttaatttatcagtattttggcagctgcagaagtaaagaattggatagcttgaaacccacaaggcaaaagttctagtcttgtttggttaactttccagggagcccaaaattttgtgaaataagagaaatgtgtaccttgagttttatgtatcctaattcacttcttgatggtacctgcaagactgtagctttgggtgatagcaaaccaagatacaataaacagagaagttcttgttttgaccctttgataattggaaattgtactgatcagcagcagctttgtggcttgagttggggggtggacaaggctaagggaagaagagggggtactgtttccaatttgaaagcagttgtagatgtagacaaaagagtggagagctatggcagtagtgatgtagaaggaaatgagagtggcagctatgatgccattgttataggttcaggaataggtggattggtggcagcgacgcagctggcggttaagggagctaaggttttagttctggagaagtatgttattcctggtggaagctctggcttttacgagagggatggttataagtttgatgttggttcatcagtgatgtttggattcagtgataaggttagttgtctaaatacttgctttctgttatttaggacataacacagaatacttgctttatttagacagaaagcaagtatttagacaagtacactaaatatcacgaatcgatcaacaagtacacaacacacaccgctccatagtctgaaagtgtggaagtgattatgttttcattcaagaatgcatccaaatattacgtataatgactacatctttaacaattttttttaatcaagtatggttttcattgataataacaaggccaacttggtcatatacaagagatataccaaaaaggtcattaacatagtttatactggccatggtgtatgctttttactattgtatatggtctgcagtgcttccacgtgattggactactgttgttcctggttagatttaaactaatgtctcacatcttttgacagggaaacctcaatttaattactcaagcattggcagcagtaggacgtaaattagaagttatacctgacccaacaactgtacatttccacctgccaaatgacctttctgttcgtatacaccgagagtatgatgacttcattgaagagcttgtgagtaaatttccacatgaaaaggaagggattatcaaattttacagtgaatgctggaaggtttgtcttcattaaggtgtctgcaggtttggcttaaattttggactccttgatttagactcaaatcatatgatgaatatggatgcacagatctttaattctctgaattcattggaactgaagtctttggaggaacccatctacctttttggccagttctttaagaagccccttgaatgcttgactcttggtaagtttttccttgctttaacttcaaatatagtattccttgaattcgcacatatccattggttggactaattcagtaataagtccccaatcatggagtcactttttatccccaagggcttagttcaagtagcaaagtttgtgggacttgtgactttggtcaccggtttgagccctgtggcatgcgaacaaagctagttatttaagtgaagaatggtaaaggggaggggccattatccccgagttttgagcactattgatggtcctgagtgtttgccctcggtcatcaaaaaaatttaaggagtcatctttcacgctgatgtgtgcagcgcgcgacgtgcttaattatcctaccgtagaatcttaatttatgccatcattattcattacagcctactatttgccccagaatgctggtagcatcgctcggaagtatataagagatcctgggttgctgtcttttatagatgcagaggtgaggcgaataatctgtgttacttattatcagctccaatgatttctttacctcttaaagaattcaactccattgtctcttttgcagtgctttatcgtgagtacagttaatgcattacaaacaccaatgatcaatgcaagcatggtaattctgcatattaaccttaggccgtgttgtgtttcgtgatattcagttccacttcctcgtgagttttctttctgcaggttctatgtgacagacattttggcggaatcaactaccccgttggtggagttggcgagatcgccaaatccttagcaaaaggcttggatgatcacggaagtcagatactttatagggcaaatgttacaagtatcattttggacaatggcaaagctgtgagttttctgtcgtaagactatttaacttttcttatgattgattattcccatcataaaatgcaaaagctgtagtctggtgtttgtgttagaaagttgtagccgacactcttttgatgttcaaggactcatgcttcacacactcataactgtggtaaaatcttcttcccatgtgatacgcctttggtgtccctaaatgcatgccgttttaaaatttaaggttacattagcatgtcagagtgttaaaacattataagaaaagaattggctagtaagtatatagaaaaagaagaaaagaatgaggagaacgaagagggaagaacaaattacttctgttaaaatgtcatgcatttgttatagttgattgtaattgatatagtgtactattttgcttatcacaacattgtgtagattcacatcccctttaattgtatttcagttacatccctgaagttattctactgatgtagggtctgtacttttactgttctacatacatgaataaattgtacctaacaatataatgacattgtatgttgccatgtcataatggtgtcttgtgtagcatcttcaatgttgtctttgccagtatctgcgcaagttcattttctctaattcttctttttctttaggtgggagtgaagctttctgacgggaggaagttttatgctaaaaccatagtatcgaatgctaccagatgggatacttttggttagtttacaaggaatccagcaaaacttatatgttttcttaagattctttcgtcttagggccagtactgtggtattcttgcacatattntgattctctttaaatctcgtcatctcatgtcaagtgtcttacatctgtaggaaagcttttaaaagctgagaatctgccaaaagaagaagaaaatttccagaaagcttatgtaaaagcaccttcttttctttctattcatatgggagttaaagcagatgtactcccaccagacacagattgtcaccattttgtcctcgaggtactacggcagttcaattgatttgctatttattttttcatacttgatgttcccattcatgattccttgattttacaggatgattggacaaatttggagaaaccatatggaagtatattcttgagtattccaacagttcttgattcctcattggccccagaaggacaccatattcttcacatttttacaacatcgagcattgaagattgggaggtaaattcagtacactccttgagtgttgttggtagtaacctcttccacatctatgtctctttttcctttttatggaaattaaagtatggccctttatccttactagggactctctccgaaagactatgaagcgaagaaagaggttgttgctgaaaggattataagcagacttgaaaaaacactcttcccagggcttaagtcatctattctctttaaggaggttaagttcgtgattttatgaactcaatagttgttcataatgagcaatattatctgtcttcaatagcaaatccacatgctcttatgcttgctgaaatagttttggccgtggagttacaccatctatgtttacaattgaattcttgtaggtgggaactccaaagacccacagacgataccttgctcgtgatagtggtacctatggaccaatgccacgcggaacacctaagggactcctgggaatgcctttcaataccactgtgagttaatcatcctttacgtatgtagttgcttttattgktctgctggtacaaacagaataaactgttccgtaatgtttctgagcttacaggctatagatggtctatattgtgttggcgatagttgcttcccaggacaaggtgttatagctgtagccttttcaggagtaatgtgcgctcatcgtgttgcagctgacttaggtaaacatgatagtccaaatagttcattttctgagcttgaaaatcctaataacatttgtgcaatatctatttacacatcataagctcgaccaaatatttataatgtggccttcaaaaatgaaaatgaatgagtatttacaacgtcgagcatcaatagccccttaaatttgctagtaaatttcatttagacactctaactataacatattacaattgagcacgtgaacacatgataaagcgcctgttatataagataagtcaaccacacaaaatatgtgacctcctccttcaactatacacatcagctttaattggaacaagggagaaggtttatggcttartcaaagggttgtcsatgtggttccggcctttaattttgaaagttccattattaaggtaacctgtctgtttggtttgacatcttctcttttttctgtcacagggtttgaaaaaaaatcagatgtgctggacagtgctcttcttagactacttggttggttaaggacactagcatgatatctacttgttaagctaaaaagaaacatttgacacaaaaaaagaaagttcattatgtatttactttttctttatatcaataaactggaaaatgaggttccaattgaggttcctccagtatagaaatcacagattttaattttgcaaattctgctaattgtcttctttttcttttctgcaactttctgccaaaggatgtcaatctcttagttatcatgaatttgatctccacctttgcattacatataatgcatccctttatgctcattaaacaagagacattgactttacatatgtatacaagttttggtgttctataatcaaaagaagatattgaaactttaattgccataactaagtgttttacatataagttacatatattaagaatatatcaaaccaaaaacaagactgaagtatagattgctttgtgcagtgcttaaaattcaatgttaagctgaaaagaaacagtttttagctaagttctcttgttcacaaacagattcataaattttaaccaaacagaggtgacttcaaagacatgtatattgtacctgttgtgcaaacatcagaagattgttctcaaccttctcaagaatagcatcagatggtgcaaacaataaaaacacaggaaactatcatttatattcccaatcctcgcgaatagtggtgagggcaggattttcactaatgaggtctaaaatataaagaagtaaaaacacacgaagaagttatagggagattcattgtctacaactgaaccttgtttatactgtgtaatttcagtccatctggtaacaaccagatccatagttatgaaacaaaaagaaaattcccagatatatgttgtatgttacattgatgcagaaaatgacttattaaaacatagctactctagtcccctactgaggctgaatcaataacaatacaccmttcagtacagttgcaactattatggttcattaatatcaaaatgttaccatcatgtcagcaagttaccttagcaatcaagtttaaaaatctcaagaagatccctaaaaatgaggtactcacaagaaatggtgagacaattaactaatgaactctggattatcctgcagggtctggctcgtcatttcacatcgatccaaattctacctctgcttggaatgcggtaaccaaaggatccaagaaatggatattatttcccccggatgtggtgccaccaggggttcatccaagccctgacggtgcanaantagcaagtcctgtttcaatcatagaatggttcatgaacttttacaacgcaaccaagaattggaaaaagagacctatcgaatgtatctgcaaggcgggtgaagttatttttgtacctaatggatggtggcatttggtcatcaattnanaggattcaattgccattacacagaacttcgttagcaggtatgctacttttgtctcctatatttctgtcacttgataaaatttgatattgttttnatgcttgctgacaaataattatatgtactagtttccttaatagcatgtaatggttctgttttataaggtggacactagatataatgcaaaagttcactattggtttagtagctgtaatttaatatcacatatactctcaggttaagctcattaactagaaaaacgaaccctcaactcacgaatgtttgcttgtttcga

SEQ ID NO: 18 - DNA, 7717 bp, Lycopersicon esculentum
  misc_feature: CrtISO genomic DNA sequence, gene tmic
tctagagtcctgaatcaatcaaaataacattttttacccaaaatcaccaaagaaaatccaaatcaactcaacccaacaccaaaattcaatcaagttcatcctcatatcttcctcagtatacacaaatttatacagagacaatcagattctactcagaaccgcattcttacttcaaatcccaatacaggttgaacagattagttaatcctcagcagcaaatctaatactaacacatatggcccattacacctaaaattttaacctatgatctaacataatcttgaacacccttttgccacttcactataacaaagggattcaacacttcatacatataaaaaaactaatcttttctctattcccacaatatagttttcaaaaacaaattagtcgagatacacgcaaattaatcaccagaaatgtacaaaaatgtaaagagggagcaaaagggttacctgaaattggatttggggaaacaaaaaaatgaatcttttgggtgtaaagagatcgcaaataggcaaaagacagttaattgcgtatagccatgtggatccaaattgtttggcgcttcttttgtaagcacaaagctaacccaatatgggataatataatttttattcctgtccaaacaaaaaaaattcttttaaattttgatcttagattaaattaaaaaaacatgagaaaaaaaaaagaaaagaaaaagaatatgcttttcactaggtgtttggaattttgcttgtagcttcgtttattatttataatacaatatgaaatcattataatattggatcggtaattattgttatagacgaacaaaactagtttatatgaatcacaaataatattacgggcctaacatgatattgaattagccgtttgaccaccaatgctccgaatgaggggtaggattctttatttttttctcgaaaagttatttatgtgaaatatattcctaacacattgaaactatttcgaaatctaaccaaatatgttagtggagcttttgacattactccgcgtccgtggataagattgttaggagaaagaatgttgaatataaagtcgaatcaatctgaattcacctcctcacgtaagattactatagaagaaattttttcctattaaatattatgtaaagtcaaaacattttttgcatattcacaatccaatttctcccacctataggagaaacctaaaagataactacccattaaccaaagccagcaagcaccaaaaatttggccagcaaaaaaataccatattgtctcattccactttgaatcacaaaagtagaatcatgccaatccacttctctttccatttcaagcactacaaacaagggaagaacccttcactttgctcatacccacctcatcaaaatccacctaaatacttctctcactttgctcaatcatctatatagcctaagaattgtcaagattccatttttatagttgttggtgtctgtaatcttggaatttgaacaatttaaagtgaaaagattcaagtttttagaattttctctgcttttgcagttgcagaggtaaagagttgtcaagattccatttttgtagtttattgtgtttgtaatcttgggtttccagcaatttaaagaaaaaaagattcaatctttttaatttatcagtattttggcagctgcagaagtaaagaattggatagcttgaaacccacaaggcaaaagttctagtcttgtttggttaactttccagggagcccaaaattttgtgaaataagagaaatgtgtaccttgagttttatgtatcctaattcacttcttgatggtacctgcaagactgtagctttgggtgatagcaaaccaagatacaataaacagagaagttcttgttttgaccctttgataattggaaattgtactgatcagcagcagctttgtggcttgagttggggggtggacaaggctaagggaagaagagggggtactgtttccaatttgaaagcagttgtagatgtagacaaaagagtggagagctatggcagtagtgatgtagaaggaaatgagagtggcagctatgatgccattgttataggttcaggaataggtggattggtggcagcgacgcagctggcggttaagggagctaaggttttagttctggagaagtatgttattcctggtggaagctctggcttttacgagagggatggttataagtttgatgttggttcatcagtaggccaacttggtcatatacaagagatataccaaaaaggtcattaacatagtttatactggccatggtgtatgctttttactattgtatatggtctgcagtgcttccacgtgattggactactgttgttcctggttagatttaaactaatgtctcacatcttttgacagggaaacctcaatttaattactcaagcattggcagcagtaggacgtaaattagaagttatacctgacccaacaactgtacatttccacctgccaaatgacctttctgttcgtatacaccgagagtatgatgacttcattgaagagcttgtgagtaaatttccacatgaaaaggaagggattatcaaattttacagtgaatgctggaaggtttgtcttcattaaggtgtctgcaggtttggcttaaattttggactccttgatttagactcaaatcatatgatgaatatggatgcacagatctttaattctctgaattcattggaactgaagtctttggaggaacccatctacctttttggccagttctttaagaagccccttgaatgcttgactcttggtaagtttttccttgctttaacttcaaatatagtattccttgaattcgcacatatccattggttggactaattcagtaataagtccccaatcatggagtcactttttatccccaagggcttagttcaagtagcaaagtttgtgggacttgtgactttggtcaccggtttgagccctgtggcatgcgaacaaagctagttatttaagtgaagaatggtaaaggggaggggccattatccccgagttttgagcactattgatggtcctgagtgtttgccctcggtcatcaaaaaaatttaaggagtcatctttcacgctgatgtgtgcagcgcgcgacgtgcttaattatcctaccgtagaatcttaatttatgccatcattattcattacagcctactatttgccccagaatgctggtagcatcgctcggaagtatataagagatcctgggttgctgtcttttatagatgcagaggtgaggcgaataatctgtgttacttattatcagctccaatgatttctttacctcttaaagaattcaactccattgtctcttttgcagtgctttatcgtgagtacagttaatgcattacaaacaccaatgatcaatgcaagcatggtaattctgcatattaaccttaggccgtgttgtgtttcgtgatattcagttccacttcctcgtgagttttctttctgcaggttctatgtgacagacattttggcggaatcaactaccccgttggtggagttggcgagatcgccaaatccttagcaaaaggcttggatgatcacggaagtcagatactttatagggcaaatgttacaagtatcattttggacaatggcaaagctgtgagttttctgtcgtaagactatttaacttttcttatgattgattattcccatcataaaatgcaaaagctgtagtctggtgtttgtgttagaaagttgtagccgacactcttttgatgttcaaggactcatgcttcacacactcataactgtggtaaaatcttcttcccatgtgatacgcctttggtgtccctaaatgcatgccgttttaaaatttaaggttacattagcatgtcagagtgttaaaacattataagaaaagaattggctagtaagtatatagaaaaagaagaaaagaatgaggagaacgaagagggaagaacaaattacttctgttaaaatgtcatgcatttgttatagttgattgtaattgatatagtgtactattttgcttatcacaacattgtgtagattcacatcccctttaattgtatttcagttacatccctgaagttattctactgatgtagggtctgtacttttactgttctacatacatgaataaattgtacctaacaatataatgacattgtatgttgccatgtcataatggtgtcttgtgtagcatcttcaatgttgtctttgccagtatctgcgcaagttcattttctctaattcttctttttctttaggtgggagtgaagctttctgacgggaggaagttttatgctaaaaccatagtatcgaatgctaccagatgggatacttttggttagtttacaaggaatccagcaaaacttatatgttttcttaagattctttcgtcttagggccagtactgtggtattcttgcacatattntgattctctttaaatctcgtcatctcatgtcaagtgtcttacatctgtaggaaagcttttaaaagctgagaatctgccaaaagaagaagaaaatttccagaaagcttatgtaaaagcaccttcttttctttctattcatatgggagttaaagcagatgtactcccaccagacacagattgtcaccattttgtcctcgaggtactacggcagttcaattgatttgctatttattttttcatacttgatgttcccattcatgattccttgattttacaggatgattggacaaatttggagaaaccatatggaagtatattcttgagtattccaacagttcttgattcctcattggccccagaaggacaccatattcttcacatttttacaacatcgagcattgaagattgggaggtaaattcagtacactccttgagtgttgttggtagtaacctcttccacatctatgtctctttttcctttttatggaaattaaagtatggccctttatccttactagggactctctccgaaagactatgaagcgaagaaagaggttgttgctgaaaggattataagcagacttgaaaaaacactcttcccagggcttaagtcatctattctctttaaggaggttaagttcgtgattttatgaactcaatagttgttcataatgagcaatattatctgtcttcaatagcaaatccacatgctcttatgcttgctgaaatagttttggccgtggagttacaccatctatgtttacaattgaattcttgtaggtgggaactccaaagacccacagacgataccttgctcgtgatagtggtacctatggaccaatgccacgcggaacacctaagggactcctgggaatgcctttcaataccactgtgagttaatcatcctttacgtatgtagttgcttttattgktctgctggtacaaacagaataaactgttccgtaatgtttctgagcttacaggctatagatggtctatattgtgttggcgatagttgcttcccaggacaaggtgttatagctgtagccttttcaggagtaatgtgcgctcatcgtgttgcagctgacttaggtaaacatgatagtccaaatagttcattttctgagcttgaaaatcctaataacatttgtgcaatatctatttacacatcataagctcgaccaaatatttataatgtggccttcaaaaatgaaaatgaatgagtatttacaacgtcgagcatcaatagccccttaaatttgctagtaaatttcatttagacactctaactataacatattacaattgagcacgtgaacacatgataaagcgcctgttatataagataagtcaaccacacaaaatatgtgacctcctccttcaactatacacatcagctttaattggaacaagggagaaggtttatggcttartcaaagggttgtcsatgtggttccggcctttaattttgaaagttccattattaaggtaacctgtctgtttggtttgacatcttctcttttttctgtcacagggtttgaaaaaaaatcagatgtgctggacagtgctcttcttagactacttggttggttaaggacactagcatgatatctacttgttaagctaaaaagaaacatttgacacaaaaaaagaaagttcattatgtatttactttttctttatatcaataaactggaaaatgaggttccaattgaggttcctccagtatagaaatcacagattttaattttgcaaattctgctaattgtcttctttttcttttctgcaactttctgccaaaggatgtcaatctcttagttatcatgaatttgatctccacctttgcattacatataatgcatccctttatgctcattaaacaagagacattgactttacatatgtatacaagttttggtgttctataatcaaaagaagatattgaaactttaattgccataactaagtgttttacatataagttacatatattaagaatatatcaaaccaaaaacaagactgaagtatagattgctttgtgcagtgcttaaaattcaatgttaagctgaaaagaaacagtttttagctaagttctcttgttcacaaacagattcataaattttaaccaaacagaggtgacttcaaagacatgtatattgtacctgttgtgcaaacatcagaagattgttctcaaccttctcaagaatagcatcagatggtgcaaacaataaaaacacaggaaactatcatttatattcccaatcctcgcgaatagtggtgagggcaggattttcactaatgaggtctaaaatataaagaagtaaaaacacacgaagaagttatagggagattcattgtctacaactgaaccttgtttatactgtgtaatttcagtccatctggtaacaaccagatccatagttatgaaacaaaaagaaaattcccagatatatgttgtatgttacattgatgcagaaaatgacttattaaaacatagctactctagtcccctactgaggctgaatcaataacaatacaccmttcagtacagttgcaactattatggttcattaatatcaaaatgttaccatcatgtcagcaagttaccttagcaatcaagtttaaaaatctcaagaagatccctaaaaatgaggtactcacaagaaatggtgagacaattaactaatgaactctggattatcctgcagggtctggctcgtcatttcacatcgatccaaattctacctctgcttggaatgcggtaaccaaaggatccaagaaatggatattatttcccccggatgtggtgccaccaggggttcatccaagccctgacggtgcanaantagcaagtcctgtttcaatcatagaatggttcatgaacttttacaacgcaaccaagaattggaaaaagagacctatcgaatgtatctgcaaggcgggtgaagttatttttgtacctaatggatggtggcatttggtcatcaattnanaggattcaattgccattacacagaacttcgttagcaggtatgctacttttgtctcctatatttctgtcacttgataaaatttgatattgttttnatgcttgctgacaaataattatatgtactagtttccttaatagcatgtaatggttctgttttataaggtggacactagatataatgcaaaagttcactattggtttagtagctgtaatttaatatcacatatactctcaggttaagctcattaactagaaaaacgaaccctcaactcacgaatgtttgcttgtttcga

SEQ ID NO: 19 - DNA, 1506 bp, Synechocystis PCC6803
atgactgtttccccttcctacgacgcaatagtaatcggctccggcattgggggattggtcaccgccacccagttggtatccaagggtttgaaagtattggtactagagcgttacctcatccccggcggcagtgctggttattttgagcgggaaggttatcgctttgatgtgggggcctcaatgatttttggtttcggcgatcggggtactactaacttattaaccagggccttagccgctgtgggacaggcattggaaactctccccgacccagtgcaaattcattaccatttgccgggggggctagaccccaaagttcatcgggagtacgaagcttttttacaggagttaatcgccaaatttcctcaggaagcccagggtatccgccgcttctatgatgaatgttggcaagtatttaactgcctcaacaccatggaattgctctccctggaggaaccccgttacctgatgcgggtattttttcagcatccgggggcatgtctgggtttagtgaaatatttaccccaaaatgtgggggacattgcccgccgccatatccaagacccggatttactgaaatttatcgatatggaatgctattgctggtccgtagtgccggcggacttaacccccatgatcaatgccggcatggtcttttccgatcgccattacgggggcattaactatcccaaggggggcgtgggacaaattgccgaaagtctagtagctgggctagaaaaattcggtggcaaaattcgttacggagccagggtaaccaaaattattcaggaaaataaccaggcgatcggggtggaactggccaacggtgaaaaaatttatggccggcgcatcgtctccaatgctacccgctgggataccttcggggcgttaacgggagatcagcccctacctgggaaggaaaaacggtggcgcagaaattatcaacagtcccccagtttcctcagcttgcatctgggggtagaagcggatctattaccagaaggaacagaatgtcaccacattttgctggaggactgggatgatttagaaaaggaacagggcaccatttttgtctccattcccaccctcctcgaccccagcttggctcccgacggttaccacatcatccacaccttcacccccagttggctcgaatcctggcaaaatctttccccccaggaatacgaagccaaaaaagaagccgattctggtaaattaatcgaccgcctggaagccatttttcccggacttgatcgggcactggattatatggaaatcggcaccccccgtagtcaccgccgctttttagggcgacaaaatggcacctatggccccatcccccgccgtcgcttaccgggtttactgcccatgcctttcaaccgcaccgccataccgggcctatattgcgttggcgatagtacttttccaggccagggtttgaacgccgtcgctttttctggttttgcctgtgcccatcgcctagcggtagatttgggggtacggtga

SEQ ID NO: 20 - protein, 501 aa, Synechocystis PCC6803
MTVSPSYDAIVIGSGIGGLVTATQLVSKGLKVLVLERYLIPGGSAGYFEREGYRFDVGASMIFGFGDRGTTNLLTRALAAVGQALETLPDPVQIHYHLPGGLDPKVHREYEAFLQELIAKFPQEAQGIRRFYDECWQVFNCLNTMELLSLEEPRYLMRVFFQHPGACLGLVKYLPQNVGDIARRHIQDPDLLKFIDMECYCWSVVPADLTPMINAGMVFSDRHYGGINYPKGGVGQIAESLVAGLEKFGGKIRYGARVTKIIQENNQAIGVELANGEKIYGRRIVSNATRWDTFGALTGDQPLPGKEKRWRRNYQQSPSFLSLHLGVEADLLPEGTECHHILLEDWDDLEKEQGTIFVSIPTLLDPSLAPDGYHIIHTFTPSWLESWQNLSPQEYEAKKEADSGKLIDRLEAIFPGLDRALDYMEIGTPRSHRRFLGRQNGTYGPIPRRRLPGLLPMPFNRTAIPGLYCVGDSTFPGQGLNAVAFSGFACAHRLAVDLGVR

SEQ ID NO: 21 - DNA, 21 bp, Artificial sequence
  single strand DNA oligonucleotide
ggaggaacctcaattggaacc

SEQ ID NO: 22 - DNA, 91608 bp, Arabidopsis thaliana
gaattcgaagatcctgcctctgtgacgaaagatgatgtagaaaagatcgctgataggtgagtgcttcatttaattgtcaatttgtgtctgggatttataaacaaaagtttgcttaaacgatccttcttatatgttgcaggtacggtgtcaacaaaggagacgaagcattccaggctgagatttgtgatatttattgccggtgagtcaacaaaggatactgtgttcatcttttctcttagtgtggggaattcattagtaatctttgcatcttaatcttgatttaggtatgtaacttccgtgcttccaactgaaggacagtctcttaaaggggatgaagtggcgaagatagtcaagttcaaaaatgctttagggatagacgaacctgatgcagctgccatgcacatggaggtgctttctagattcttaatcttcatttgaattagccgttagcctttttgtagttgatgcagaaggatcttgattgctgaatcaccaattccttggttttgtttagattggtcgcaggatttttaggcaaaggcttgagactggggagcgtgaaggtgatgcagaacagcgtcgggtgagtcgccagtccctagtatttgttaagtttattgtaggattgttcgaaagagagcacaagcgtgatcacaagttgtatgcttccagtgcaggcatttatgaggcttgtatatgtttcagctcttgtgtttggagatgcttcatccttccttctaccttggaagcgagtattgaaggtcacagatgctcaggtaggcatttccaatttttttcatacttctgattttgcgcaatagttgttttatggaagtttcattttggcaactcactccttcttggtaatctgaagttctgtgctaatgtcttgacctcatttctaggttgagattgctattcgtgaaaatgcaaagcagctgtatgccgaacggttaaaattagttggtagaggtaatgttgttcatgttttacattttgagatgggaagtaatgttacatttgtagaacgggtttgagtttgcttgacttggttatattgattattgtggcacttctttgtccattgggttttgtaatattcgtttatcagcaagttgtctgaataccaaccttaattgttacttcgcatgcgactgtaatggtcccttctcatttaacattgataatagatatggctttttgacaaccaaaaaaagatatgacttagctttttcgtcagcttaatgcatggaaacttacatggttatgtttctaaacatgttcttgcagatattaacgtagaaaaccttgtggaccttagaaaatcacaactatcattcaagctctctgatgaggtactctatttctttgtggcattgacttcctgctatctctggttgtacttcttctgacagtttcttctccctcatagcttgctgaagacctgtttagagagcatacgagaaaagtagtcgtagagaacatttcatcggcactcagcatactcaaatcccgcacacgagcagcgtaaatcattttaacatattgcttacagaatcatgcatagaaaaatcgtatagaataatcattatccatgtgaacagtttggttacacatttaacttttttatttcttataccaatgagttatgactttagctgggtatatgtattcctttgcttgaacattgagtttggtcgggttgaaaaaagagattgaagttttggctttagttttgagttttggttattgcaaaaactgattggtcatatgatctcttttgcttttgtgaattgtcttatagtataagtatttaaaaaaaagtcatgagattttaattttataggcgtttttgtatcttgtgataattaatagtggaaacaaattggatcatctagtatgtctagtttatagtagtttacaatgcatgtcattttaacgacctgattttttccaggaagagtttggcatctgtcgtggaagagcttgaaaaagtactggagtttaacaacctgctagtttccttgaaaagtcattcagaggcagatcaatttgcacgcggggttggccctatttctttgataggtgaagatgcttttgaccttctcttttctgaagaaaaattattggctctcaaccttggatctatgtggcttaggtgatgagtctgattttgagaggagaatggatgatttaaagctcctttatagagcatatgttacagatgctttatctggtgggcgcttagaagaaaataaggtatactcagttttgtataagatattatgtactaaagcttagttgcaagtttttttgagagtaagtgttttcagatgctgtgtcctctatcccagtcgcatgtttttttccatttctaattgtctttctactgtatggtagctcgtggcaatgagccaacttagaaacatactcggtctgggaaaacgagaggctgaagctattagtgttgatgttacgtccaagtcttaccgtaaaagacttgctaacgctgtttctagtggtgaccttgaagcacaagacagtaaagcaaaataccttcaaaagctctgcgaagagctgcactttgatgcacagaaggcaggcgcaatccatgaaggtataagccgaaccttctgttcacaatgaaatcttatgtttttatgttatgcatgaggtatacagttggaattgccacataatcgcccaaatgtaagatgtgtccatgggtatcaatttagttgatggcttgtaaacttgcgggttccttatccttttaagctagcttttagtgttgtagatttggttcttaacagacttctaacaagtctggtgtttcagtgggttacatgctgttattttacagtttgagtatctatttggtctttttttcccaatcatctgacaagcaccttctcttcctcagaaatctatcggcagaagcttcaacagtgtgttactgatggagagctgagcgatgacaatgttgctgctttattaaggttaagagttatgttgtgtattccccagcaaactgttgatacagctcatgcagaaatctgtggaaccatatttgaaaaggtaatccagttatcacctcccttttccctttatcaagggttttttcatggtcttgggtcttggcactgcaatttccctctgcttccctctctgccttcgcattttgttaccaaaaggttttacctctaattacctttttgtttaatctttccaggttgtcagggatgccatttcttctggagtggatggttatgatgctgaaactcgcaaatcagttagaaaggctgcacatggtcttcggttatccagagagactgccatgtctattgctagcaaagctgtgagtatcacactatttactttctccatgcttggtattaggatttgttaaaattttctgacttctcatagcaagacatgaaaatcaaatcccttcattgatgcaggtccgtagggttttcacaaactatatcagacgagcaagagcagccgagaaccgtactgattcagcaaaggagctcaagaagatgattgctttcaacacgttggttgtgactgaaatggtggctgatatcaagggagaatcttctgataaggcacctgaggaggaccctgttcaagagaaagaagaagatgatgaagatgaagaatggggatcccttgaatcgctcagaaagacaagacccgacaaggaactcgctgagaaaatgggaaagcctggccagactgagataactctcaaagatgaccttcccgacagggacagaatagatctctacaaaacatacttgctctactgtgtaactggagaggtaacaagaatcccttttggcgcccagatcacaacaaagagagacgattcagagtacttgcttctaaatcagcttggtgggattctcggattgagttcgaaagaaatagtcaacattcacgtaggtttagccgagcaggcttttaggcaacaagctgaagtgattttagctgatgggcaattgacaaaggctagagtagagcagctagacgagttgcaaaaacaagttggtttgcctcagccacaagccgagaaggttatcaagaatataaccaccacaaaaatggcaaacgcgatagagaccgctgttaaccaaggaagactgaacataaagcagatacgggagctcaaggaggcaaatgtcagcctggacagcatgatcgctgtgagtctgagagagaaattattcaagaagacagtgagtgacatcttctcatcaggaactggtgaattcgatgaaaccgaagtctaccagacaatcccatccgatctcagtattgatgtggaaaaagccaaaagagttgtccatgatctcgctcagagtagattatcgaattcgctggtccaagccgtggcattactcaggcagagaaactctaaaggagtggtatgcaaatcatcttttccctctcttatcgtgacagaaaacatggatatttcagcttctgggtctaaattgtgtatgtatgtatgctttttttttttggttttacaggtcttgtcgctgaatgatttgcttgcatgtgacaaagctgtgccggctgagccaatgtcatgggaggtctcagaggaattatctgatctatatgctatttattcaaagagtgatcccaaacccgcaccggaaaaagttttgaggctacaatatctgctgggaatagatgattcaactgcaactgccctccgtgaaatggaagatggagcattatcttctgctgcagaagagggcaatttcgtcttttaaatctttggagaaatcacttgagttattatgtattgtgttacggccgagggtggaaaaatgattttgaatttgaaatttgaccaaagtctgtgaaaattttgtatcagaaccataaacttgatatgattaatttgaaaatctgcaaacgagaagtgtatcccttatacaatttagagcgcagctttgaagtgctcatgtggtgtcacgtgacgggtgttcccgcataacgaacctaaaaaatagaataacagttaattcaatcattgagaactttttttttaatgaaatgatttgaccgtaacaccgtaagtgaaagttaattgttaattctatggtggtctcacatgactgcacacaactagaacaatactcaacaatcaacatgtatactagatctgatctcgttatagtatgtttttgtcgaaatttctaattatacaaaagcacgtcttttaaagacaaaaatagaagccatatgatccaatcaattgattcatagagatgatgatatgaatcgatgaaacatagtttttagaaattatttccaagattacaaaaaaagaaaaaaaagaagtgaaaattaataagaaaataggagagagttaaatataagaaaaaaggcagaatcatttaacatgatctgagcaacaccttgacaagggaagcgtaacatccagaagatttagcactattcgatccgtagtctaaactcgaaccagattcaaaccacattgatcttcttctgttgcttaaatcagatgtcctcaatggagaagatggaactgaagacgaggacgacgaagaagaagaagccggagaagatggcacactacgattcttggaaagcaaggaactaaagagagtgttggtttttttagggcttacgtgttttgtagaggatggagaagcgtagaagtaagaaggaggaggtgttagtggtgctgaaaccggaaaattgaaatcagaatcagaattgaaaccggaagaggaggatcgtttggagagtcgtcttggagttccgggctgagattcccatttgaagggaacggcggctgatgatccaccgtagtagtcgccctcgacggccacagagaggatcagtttggtcgtggaaaccctagaacgatccagctgaggcattggacactttgtgaggaggtgacttttcttttccgggtttgctcaactatactataagagaactatgaaaagaaagagccttgagtctcttttttttgttttcttactttattgggtgaactaagaagatttagattcagattcaaaagttttgaattgtttgtgttcttattttattatatagttttgaatgagtaagaagcaatttgataaaatattgtagaatctcgaggtgtgatgcttctgggctctgcttcttggaagccactgagagtattatcctgttccagatcatctgatccaattataacttccaagacacttggtcgatcctacccctagaactctgttttgtgagtgattgtttgatttctctactcatatcttttattttagtattagaaaacatatttctaacattttaattatataagttatagttataacatttaattaatatttcattctcaacgtacgttacaaagttacaaaactacatttaagaaatagcataattagtaagaagatcacaatactagctttcttgctacatgaagttaggctgctactcagtttcggtcctgtaaaatcaaggtccaaatacgaaaaaaaaatctgaatctttttaaaaaatttaaacttgcaaaaatcttttttttcttctcataattatgaaaattatttaaagagttttcagaaagtaaaaataatatatccgagacatgcaatcctattagactttgaaagagttttttcaatgacattgtatgatgtaaaaaatactaacataaacgatcactacacacaagaaaaaaaactttaaaaaatacactagaattggtgcacgggaaaggccgattgaaaataatctaggcccattaaagcgaagcccattctcaaccctatatatttgcggggttgatatttaattttatctttcaatcagggatttcgaaaccgaaccgactctgcttatctctcaggtaaatttctctgctccttatctcttcatgtttctttgcaaattcatcgactaattgcatagatagcctacaacttatatatatatatatattctgaatttgtttttttcttttcttacctttaaggccttgaatgatggagggaggcagctgtggccacaatatcggtctattgggagataaaggggtgtccggttccggatggctatgatgctcttcgggtaggtccgtctataaaacgtaatttgaggaagtttaactacactggccctatcaccatcactgccgttggcgtactatcagaggtccctcgagatttccttgaaaccactggagagtggagtcagtctctttctcaagtcttagttgtttctgaaacaggtcccacatacatggtttcgcttttcattgactcccctgattctaagattccacgtccacataatacctatgaagacgacgaggctcccccttgattattttccggagaaataatgcctactaccattggtgagggaagaagcaaccaaaccttgagatttctcatcttttgaattctggggtttaccggaaatcttggtaaatagaaatgtgaaatgtaccatcctttaataatcttttcttattatcttttatttccgtgatttaatgtagtaaaatcaagttcctgtcacttgaagttgtacaacttacaaaaaaaaaacagaacacagagaaagctactctgtatccaatgctctgtgttatcatgcatatattataaaacccatttcaatgatctttaatcgtcatggtcgcttggaaacaggtcagtgcagagctctccatagaatctagtcacaagatcaatgaagattggactattaagattcttccaataatagagaagctcttccatgtccatcaagtcatctattttcccatcttcaacgaacatatggatcatatagttctcgaatcttctgcaagcctctttcacaccatcttcctttaacatatgtctttgactcgtcctcgctttcttgttcctttgcgtcatagccaaaggaaacatctttcttgaatgcagaagcttggaatcatcgatcagactagctgcagtggcatatttgtagaataaaatttagatcaaaggtactctataaaatcgacaatagttacaaaagaaattgttgttacacggggaatttctgggtctgatctggctcatcctctgctccttattagtattgagaaaacagagacgagacaacaaagaagatgatgaagattgtgtggtattgacattgcgaaaacgagcatggaagaaggagatgaatctgttgcatgattttgaaagctttgatttcaagctaatcaaacttgacttcagtctcattctcgatccttccgaagctgatttattctttgttgttgttgttgtggtataatgtgtaggtttgggagggtggttcgagttacgtgggtttcacgtcttttcctttgacacgaagtcgtgatggtggagtcccatgaacttgagccttaatttgaatgttttttgcaaatttaattttaagctataatctgacctagatattgtcacattatgagtaggaggagtgggtaaatatatcggtgtttactgggatcagtagtagtcgttagggttgtgactgaccccgctcgtttgtcgaaccctagcttcaaatatgagtcgttgacccaaaaaaaaaaagctttggtcaaacacacacgtgtgaggtagggagaccacaagctaattggtgaaaaggagacgacacgccgagttttatgcaaatatgtcttttattattctctacagtttcttctacaccattattgttatatactatgactatggctatattaagagattggacatgaacctgaaatcttgtttaacctcttttgttgaaaccctttgtcacgttcgccgaattatgttttggaagcaatttaccagaaatattctatttagtctaattgatacccaccaatcgacctactaccgacagacaatttttgaggacactttgttgctcaattttcaagtctaataaaaccttttaattctcaatctcatctacgtacacctcaattatttcgattacaccggtttagataggatattttatttaaatttgatttgatttggtttaacttggttattcggttagggagagaagccggaatgagtttggtacaaacaaagacattctatgaagtggatcccaattttgttccgtcttgtcccttttacatcccaattctacactttcttctctcttttatttgtcacatagaaactctgcaaactatacatattttttttctctcttttatctgagaagatcacttattttttgaaattagagatgaggaactataagttaagattgtcagttatgagcccaagtgaatggttccacaagctcaagaacatgacaaaacctagaaaaaagcattctcttcctctttactccataaacactaccaagaagagaaaaccttcctctgagtcaaagtctcttccttactcttcaacttcttacttcttcaataggtcacgctcacgcacatcctttgaatctaggatccttcaaatttcaccaagaaactctcttcacaacatacagagcaaaagaaagactgtttacaagccttctcctccttcctcttctattgtatctgcaggttttaacaaaacatttcatcaaagccatgattctctctctgcctcttccaacttgaaagtcatttcttctgaagatgatattatcatcgacatgaataatagagatttcaagaagaaaactttcaaagagatcacgaagtttgattcaacggagaaagcctgtcgtgcaagtaaccgaaccaaggaaacacatatacctcatcatctctctgtgaaggtcagtaaagagaaagaagatgaagaggaagatgcatgtcgaaccaagaagaaacatcagaaaacattagtttctagtggaagaagatcatcagctaaatctccaaggataaagctcagagcgagatctcctagaattcaagtctcgcctcgtagaagcaaatcaagatcacagaacaaacaaattcttgacagttttgcagtaatcaagagttctatcgatccgagcaaggatttcagagaatcaatggtggagatgatagcagagaacaacatcagaacctctaacgacatggaggatcttctagtatgttaccttaccttgaatcccaaggagtatcatgatctcattatcaaggttttcgttcaagtctggcttgaagtcataaactctacatttgcatcgaagtagaatgaactatgatcaaaagctttaaatctctggttaattgtagttcttttaaagagtttgatgtagccgtggagattttctttgcaagaccagtttttgtgttgtctgaataacgtgataaagacatataaatcactcgcttccttttcgtctctggttatggtttgtgaaggtaatagaacatacttattcagtaatttaaaaatcagtaactaaaacatacttattcaatacatgcctcatggatgataataattcaattttagaattaaaatgtgaaacagtatgtgctgctaaatgtttatgtgatattttcagaaacaatatacaaatccaaagagttttattatattagtttcatgtagtgataaaccttggattagattaggattgatggagtttagtcatggtaacgacttatgtaacaagagcgaaaatgtcaaaagaaaaaaaaaagagtcaagaaaaggccaaaagtgtagtgacaagaagccacacggagacaagtcagaaacccatcaagtacggagtagaaatgggttccagtcctaatgagtaatgaagcaggaaacatgaaaatggagcctgaataacataaatcgttagagagagaaactaacactgacacagaagagtatatggttaaagaggaaaagcagagagagagttttaagaacctaaaggctttttgttgtcgtgatgaagaagatgaggaccactgttttgctgctgcttcccaatgcccatctcctctaccatctatttatactggtagtacttagtacggtatggacagacatttcttgggaattgatcaaaccacaaaacataaacattaaatgatgaaacaccaaactcgattttgatatatagtgcacgggaaaggccggtttaatacaaactgtaggcccattaaagcgaagccccattatcaacgctatatatttgaggcgttgtgattcttattcttctctttcaaacaaggatttcgaaaccctaatcgatattgaggtcaagattcaaacaactctgcttatctctgaggtaaatttttcgaaaccctaatcgatatcgactcattgcgtaaatgaaacgccaagacagtagaaaacgtaatagaaaataatatgaaaccgaaatgaactgctcgtctacatttgactaaggaacctagtttacttaacaaaaccattcactagacaatcatcgtcgtacgctattctatgggtcttaaagacaaaaggctacaaaatgttacccagtttttttacaatacgtcattgttcagcagttgttggtactcgtctactcacaaaaaggtctgatgcctactccagtactccaatacctataagcaaagaatggaaaattgatggagcaattatacgtaagattaagaacaacgagagatataattatgattttaatgccgaaaagaaagtaagaagatcatcagctattaaaacaccaaccctcctaagctcaactaacccattataaagttagactaccatgatgggtgagcattgatttgtttacccatcaatttatcacatcagtggtcaaatatatagaaatcataaaatccattattcctgcatattcaatttctcccttctacagaatatgactcgttgacatagtaaaaaaccaacattgatagtattttatatgctgtattaagaagcattatacatacgctcatagttctacaggtagagtttaccttcaaagctaacttgtgtaagcaactattggcttcttagaacttggctcactatttgtgagggttcatatcattacccctcttctactctacacactttagtttcggggaactgagatgctaatgcagcagtactttattgttattctcttctaaaggtcaccagggtgacgattgtaccttatagactatgactatgtttaactcacctactcaatgacggcgctcttcacgtctcatctcaactcgaaatttatctttaatatcaccctgtgagagaatgtgtctattattagaaacaagataaaattaactccgagaaatgaataaagaacagaaagataagtgaagctgtgtacattagttctgcgcactagaactggattctcatctttgatccgtttccagtttgcaccatacctgaatcacaaaagaatgcatcaaatcaatagaaatgcatgaaagatattcatctaacatctaggagaatcttcaggtttcagtacttttcatagcctttgagtacggccagcgtttctgcagtactccaaggaagttttggccttctagcaccttctgaacatcggtttcttttcaaaggagacacgacaattcttttacgttttgatttattaatcctttcaatgtcatcgcccatttctccatctgaatcatcaatggagtcattccactaatcaaacaaacaaaaataatgtgaaaaacaaactcatctgaacaaacaagtcctatgttatacagttgcatgaaaagcacattatattaactcagctataactccgaaaccgaacatgcaaagaaatcaattcagcgagtcctacacaatactaattcccaccgagtgcagcgggctggtcagacaatatgaaataataggaaaagatcaaagaatgaatcgggatagtgctgagaaaacttagttaatctgtgtgtattgggtgggtattccatcgacctagctatttaaacacaggaagtctgaagaaaaaaacataaatgatatgatgaaacaattagaaaaagaagcaaatttacttcataagtgtgtgctgtgctacgtggctccatcaagctaggccttggtgcagcgtttgttgcagaaggattagcaacatcgttttcttgttctgtaatcgtctcattgtttaaatttgaaggtcttcctttttctaaagctttcattagctcgattttactagctctcagctttctgagcgctctgtcaacaactgcagatggaatgggattgaattgactgggtacatatacagcttttgaccctccagaagtaggtctttccagttcagtatctaagaggctctctacaacaaaaggaatgcatcagaccctttcagtatccacaaaagcacaatcacatcgtaacaaagtaaatagagatgaaagaaacaaagttgagaaagtgtagaccttggtcgtcttcttccatagcttcagacgcctgactgttctgattacccacgccctctcttgcttcaaccattgattctgtctcttcagattcttcactagattcaatctcatccgattcatccatgttgagattctctaaaacaagattgggattcaaatctaaaacaaccttcctcaaacaaaccaaagctttgtctcttgtatcttcatccatcagtgtcttacaagccttgtcgtcagtgtgggccttccataatcttcgacagcatttcaagaggtcaagagtaaccaaacaactaaccttatcgcataaaggcataatcctaccgagccatatcgttttgattgcttcggtgtaagccttttttgcatctttctcataagcgagacacttcactgtgcactccacagctactttacaatacgcttccgtgactgatttgggtactttagaaccttctttgtgtaaaatctccacaagattctctaatcgctcgagattcttctcttccacaataccatcctcaatctccatgaaaatcccttcgagtatagcttccttccactgcgagagtgccattgttgctaaatcaaccaagcaatgtttgggctttttagagaaatcagtactttctcactcgccgattccgtctctcgaattgggttacacaggaaaggtcaattaggtttgtaacacttcaaatttccaatttttcacttttcgattttcttccgattacagaggcgaaaacgaagcgatggaagaaagaaccataactttttgccgtttccatctttaaaaatagaaacgcgtgtcgttttatttactataaacggctcacattcaatatccctagggagaaaacggcaagatactcactgcttctaactttttacattttttaataaacgaaaaatcatcagaaggcatttgcggcggaatcggattcattgggtacagagaagcaagacctaagaagaggaagctgcagcagcagcagaacttgttcaccagtcgctatcttcttctcaaaacgagaacaatgtcttcaatgaaatccgtctcggctttggacaatgtagtggtgaagtcaccaaacgatcggaggttatacagagtgattgaattggagaatggattgtgtgctctgctcattcacgatccagatatttaccccgaaggctctgttccggatcaaattgatgaagatgacgaggacggtgaagaagaagatagtgacgggagctctgaagatgatgatgatgatgaagatgatgaggaagatggggaaggagatgaagaagatgaggatgaggatgaagatgaagtgaaagggaaaggggatcatcagaccaaaaaggttttcaattaaaagctatatctctgttttcttcttgtatcgggttttgatttttaaactagtatattttgtgttgtgtgtctcaggctgctgcagctatgtgtgtttcaatgggaagcttcttggatcctccggaggcacaaggcctggctcactttcttggtattttcttctttctatgagtttgaactacattttgaatgcacttcttctgattttttttactaattgaatcactctcatgtttatgtttggtgaagaacacatgctttttatgggtagcactgagttccccgatgagaatgaagtaagcattgcattattttctatggtttcaatggtttggcattatttatagattcatatgaaataagtattctgcttactttagaattttctatctttgaatcaagaaactaaccgagttagaatgattagatataacaatgaatctttacggagcttggatataatggatatggacttcgttttgttagcatgatgtatcttttaattcttccggttttgacttcttgcagtatgatagttacttgtctaagcatggtggatcctctaacgcatacacagaaatggagcatacatgctatcacttcgaagtcaagagagaatttcttcaaggggccttgaaaaggtacaaaaattgcttgagctgctacttcacatatctagataaacgtcattttgctgtaaaatttaccaaggcagtcgatatttatgtcggtatgcatgcccagagattgttatcattgtaaggaagatgcatagttttttatccttcctggtcgtcattatgtaattacttgtctttcctataccagttgttgtaaaaaaattccgtacgcaatatttgagctttctgttaagtttaatcttttccttttcatttttcttattcaaaatgcttatttgaccttagaactgttttctcgttattattagattctctcagttttttgttgcaccgctcatgaagactgaagctatggaacgggaagtcctggccgttgattcaggtttatcttgcttgtttcgtactcttatttctttgtttgaaattatcaaaagatccatctccaatcaacattcagatatataaatctttctatatagttaaaagtgtcttgctaacaaaatccaaatttgtagaatttaaccaggccctccaaaatgatgcatgtcgcctgcaacaacttcagtgctatacatctgcaaagggtcatccttttaataggtttgcatggggtaaggataccgcagtgtgccttacttgccacatttaagatattcttgagaggatttattgttaatatgtccttcatgtgctctggaaggtaacaagaagagcttaagtggtgcaatggaaaatggggttgatctacgggaatgtattgtgaaattatacaaggaatattaccatggtggactgatgaagctcgttgtcattgggggaggtaaaattctctaggtgtatgatctggctgtgattcttggaaaatctttatattcattcctaaatattggcttgtctttactgtttgagtttattgtagtttaatggttacctttaatatcttgcagaatctctcgacatgcttgaaagttgggttgtagaactatttggtgatgtcaaaaacggatccaaaatcaggccaactctggaggcagaaggtcctatttggaaaggcggtaagttatatcgcctagaggcggttaaagatgttcatatacttgatttgacatggactctccctcctcttcgctctgcctatgtgaagaagccggaagactacctagcacatctattaggacatggtgagaaatcacatcgattttatcttttgtataaagttttattaagtgtgcttgtgcatgtccatattgaaggatgttgtttatcacattgtgttttttagagggtagaggaagtctgcattcattcctcaaagccaagggttgggcaacctcactgtctgctggtgttggggatgatggaatcaaccgctcgtctctagcatatgtgttcggcatgtccatacatctcactgactctggtttagagaaggtatcttgtcacatacttatttatacttaatttcccctaagtaagttggacgcaggggataattaatcattttcaattgtagatataaccctttgtctctgaattttgaagtaatatatttacttgtttcttattttggttgataagctgatatagcctgttactagctgcatatacagtagtcttgttcttgccatatttgcttttgttatgttactatgtcccatgttgaccttatggcaacttttgttaacagatttatgacatcattggttacatctatcaatatctcaagttattgcgtgatgtgtcaccacaagaatggatattcaaggaactccaagatattgggaacatggacttcagatttgctgaggagcagccggcagatgattatgctgctgagctctcaggtttgtgcttatatatagttattgtctattaacttattatgttggcatgtaaagcctaaatacttttagtacctatcatgcattgatgtctacatgtaatgtaaagacttcaacatgggtttcttagttaatcattgaaagtgttaattgtacagagaatatgcttgcttatccagtggagcacgttatttatggtgattatgtgtaccagacatgggatccaaaattgatagaagatcttatgggttttttcacacctcagaacatgaggattgatgttgtttcgaaatccatcaagtcggaaggtattttaaagattcgtcgagtatggaacttttgaaaacaaatttatatacttagtattcttcttttgaaattttgcttgtttcagagttccaacaagaaccttggttcggttctagttacatagaggaagatgttccattgtctttgatggaatcatggagcaatccttcagaagtagataactctttacatctcccctcgaaaaaccaattcatcccttgtgatttttcgatccgagccattaactctgatgtggatcccaaaagtcagtctcctccgagatgtataattgatgaaccgttcatgaaattctggtacaagcttgacgaaacttttaaggttcctcgtgcaaatacatacttccgcataaatttgaagggggcatatgccagtgtgaagaactgccttttgacagaattatatattaaccttctgaaagatgagcttaatgagatcatctaccaggcaagtaactaatttttaggaaaatcttcttagatcattttgtttttgcattgacattaatatctgactttttgcaggccagtatagcaaaacttgaaacttctttatctatgtatggtgataagctagagcttaaagtgtatggctttaatgagaaaattccagctcttctatcaaaaatcttagccatagccaaatccttcatgccaaacctggagcgatttaaggtatgtgaatatttgcatgtatagttgtcatatttttaattctccttttatcttaaatttcaagttttgtgtcattttcttatcctcatcatttcttgatttaacttctttttctttctaaccttgtaggttatcaaagaaaacatggagagagggtttaggaatactaatatgaagcctttgaaccattctacatacttgagactgcaactcttgtgtaaacggatctatgacagcgatgaaaagttgtctgtactaaatgatttgtctctcgatgatctcaacagctttattcctgaactccgctctcaggtaacattctgcatcagtgtgattcctgttttacttaatacacagattttgtgactgttctctggttttgtttaaattgggcaaaggtcatgtaatacctctgttatgttttcagatatttattgaggctctatgtcatggtaatttgtccgaagacgaagcagtgaacatatcaaacattttcaaagacagtttgacggttgaaccactcccaagcaaatgtagacatggtgaacagataacatgtttccctatgggtgccaaacttgtgagagatgtcaatgtgaagaacaaatctgaaacaaactcagtagtcgaggtaaaaaggaaacagtattttcttgtgataagagtcagccaagaagagttgaaaacattgtactttcctcgggtataatcatggttttgaattttaacctgcatctgatttgcagctttactatcaaatcgagcctgaagaagctcaatcaacgagaacgaaagctgtgctggatctctttcatgaaatcatagaagagccattgttcaatcagttgaggttagtcatcacatgtttacctagtttttttaatagatcaaaattgtattcatgtatagtcacacatatatatacatattttggatataggacaaaggagcagcttggttatgttgtcgagtgtggccctcgcttaacgtatcgtgtgcacggtttctgtttctgtgttcaatcttctaagtacggtccagttcatttgctggggagagttgacaatttcataaaagatatcgaagggcttctggtaagtctgtaatcaaaaagaatatattgattttttttgttaaatctttacctcgctccacatatgttaatcggactgtaattgcaggaacaactggatgatgaatcctatgaagattaccgaagtggtatgattgctagattgctggaaaaggatccctctctcttgtccgagacaaatgacttatggagtcagattgttgacaaaaggtaaggatctcaagggtttgaagttatgtgacctgtagtatctatagaactttcaatgatttacttggatccatatccacaacggtcactggcgaatgaaaatggagttaactctctcacaggtacatgtttgatttctcccacaaagaagcagaagaactaagaagtatacagaagaaagatgtgatcagttggtacaaaacctatttcagagaatcatcacccaaatgtcgtaggcttgcggtaagggtttggggatgcgataccaatatgaaagaaactcaaacagatcaaaaggcggtgcaggtcatcgcagacgcagtggctttcaagtcaacttctaagttttaccccagcctttgctaatgagaagaaacatacaaaagagttttgcttcgtcttcccttttttttatcttttatattttgattcactcaattttcttcataccctcattggataggttatacttttgggtgacaaacaccaaaaatatccctttttgttacgatttgagaatacgctttttaaattattatttttatctggtggataatttgatatttattgattagaaaagcattatcttatacaatgtatagatttcatttacccaaaaagaagcagcaaaactaaaaaacagacagaaaaaaaaagttatcaatcgataaaaacttacttcaaataaccatcgaaactctcactaagaaaaatcccatttggaacaattttctccttaatgacaggtatatgtccactggaacaataaatgaaaagaaagtataaaatgacctttgattgtacagtgaatgtcttacataaataaccaacggtaaaaaatcaaatgtgaacgtggatcatgaatagcttgtttgataattacgattatatcccgcggttaatttctctctggtagggcctatcaaacgtagaaagcgaaaactatctttcttcttcctctctctcactatttttctctgtacggtggtgagtgaagaccatcaaaaattgcagaaaataggcttaatctgaatctcttctcctccgtcgaagttttgaattctctctggttctccaattctcctgtgagtctctctctctctctcttccctctttctccttccttatgttctccttgtttcatttcgcgtctcttgaattttgccttcaactgatttctttgatctctgtctcataacggcagatctagggtttatggcttctggctttcaataaatctccttcgttctcccactcttttatctaacgagtttctattttctccattttgcttcagctcgagctgccttaagcttcgcatttacgtttacgttatgcacgaatgaggttcgttttctccagatcgctcttgttttcttcacgttttactgtctaccgttatcattccttgtataatttttggtggattagatcatcgtagatcgtgattgaagaagcgagagataaattaggagagcgaagatgagcgagggccagaagttccagttgggaacaatcggcgctttgagtttgtccgttgtgtcatctgtctcgattgtgatctgtaacaaagcgcttattagcacccttggcttcacattcggtaaccttttcaattcttccttcactgttacaggctcctttgattatccatgttgcgctgctgatattgtttcctctggatctattcttgtgtttgtatactaatgcttactctgcgcgattcgtttcgagccttctcttatttgatgaaattgaggaatagattgttgatcattttacttcatactatgttctgaattgtctgttggttatattttaggtcccttatggtacataaacgagaattgccttcatgattgtatatgtttaagattgtttcttatcgctttagaatttaggtgctcctcttcttttctgtatgactcaatattctttcctaggatacaccattgattcttatgtagtattattatacttgcaagacactgttttatcccattttgatgtctgtgttgccataacagcttttatgtcatcttatgcaattttgatctcataagctatctgttcttactcttgttattttgtatgtacagcgactactttgacaagttggcatcttttggtcacattttgttcccttcatgtggcattatggatgaagatgttcgaacacaagccttttgatccacgagctgtgatgggatttggcatattgaatgggatatccataggactattgaacctcagcctgggctttaattctgtcggtttttaccaggtatagctttttccatttcagttactgcatgatgtgatatccatttttaactagtgttgttctaatttctaacccatcttctgttccgttgataatagatgactaaactagctatcatcccctgcactgttctcttggagaccctcttcttcaggaaaaagttcaggttagcactcaaattctcttccattaacacgtgaataattattgatttggaggaagctgttttacatgacatctatatttttcttatggtcatgcatttctggttgacttaatccaaagttctaattttatatactgtgtaatagttgagcaacttagtgatagcgttatgtctctcgctagtttgtggtcaagggcctagttaatgacacccttattttttttctttgcagtcgaaaaatccagttttcattaaccatccttctccttggtgttggaattgcaaccgtcacagatcttcaacttaatatgctgggttctgtcttgtcgctgctggctgttgtcacaacttgtgttgctcaaattgtatcctatgcctagcctttggtttctcattgctttattctttcacctttctatatatcttgtcttaaaccaattcgatgctgttatcttaaccaattgtacacagatgacaaataccatccagaagaagttcaaagtttcatccacgcagcttctgtatcagtcttgcccatatcaagcaatcactcttttcgtcactgggccatttttagatgggctcctaaccaatcagaacgtgtttgctttcaagtacacttctcaagtagtggtgagactcaatttcccggggattctaccaaacttattaactgatcttcttgcttccatgtcgatcctgcatattaacgagtgcttctttgttgattgcagttcttcatcgtcctgtcttgcctcatatcagtctctgtaaacttcagcacatttcttgtcattggaaaaacatctcctgtcacatatcaggttctaggacatctcaaaacatgcctggttctagcatttggctatgtgttgctgcgagacccattcgactggcgcaacattctcggtattctagtggctgtgattggaatggttgtttattcctattactgctcgattgagactcagcagaaggcaagtgaaacctcaactcagttgcctcaggtatatttcttcatctctctgttccctctaatatatttctgttggtaattagatgagtccctcaaatcatattcctttttatagtaccattgattttgcaattagattgctataaaaaagcatcagtatcataggcttttttcaggtctatctatatatactgacttgttatattgcttaatttggtttactcgggtttgtcagatgaaagagagcgagaaggatccgctaatagcagctgaaaatggaagcggagtgttatcagatggcggcgggggagtgcagcaaaagacggtggctcctgtatggaactcaaataaagattttcaagcctaaaagctttgatcatcgctatttttttatataatatcaaactctctttgtattgtccccaaaaaaacacaaactttttaatttctctttttttttttttttgttccctttttaaatatgcatagcagagaagtaggggagactgaaaaatcaacaaaccttccacaccatatggaaaaaaagaagaaagtagagagtgaggggaagaaaagggaacagctttgatgatccattttattatacacagcagctctcttttttttttttttttttttttggatttagtattacaactctgtattcaaaaggctgtggctatattcatcaacactgaaaacatatatttgatttttagactcttttctatgaatctacacaactcattctagctttcttaccaacttgtcccaattcttattcagttattccatatcttgaccaaaccattttgatgagagtaaaaaaaaaggtttctggtatttattttaaaacatagcgtaaggttcaacaaagcaccagtggtctagtggtagaatagtaccctgccacggtacagacccgggttcgattcccggctggtgcatgagctgtgatgaaattggctttagtattggttgggtccaatgcattctcctgaatatcagatgatttcgcctactctgagctctatttttaatttttatttttgaaatattgcattttaagtttatacaaactattaatatcttgtatctgccaaaatagcctgcaaattccacagaaccaattatgttcaagagcttgagaacagaacgtttacaggattcttcacattaaatactcaaaaaagaaagtttacccttttatgtttgacagctactgccaattacactggatgcatttaggtttcaacacatcaaaaaaaatcaaagcaaaaatatcaattttcttgttctttttatatggatttcccagtgcattccatgtccgccatcaagcctagacaggaagaaagaattgctctgttgccttcagcgccaaaatggttggcatggcacagctcatctaatcaaattttctgcttaatcctaagataccatctgcgttggtcaagatacatcaacagctctcttttggctaacttgttcatggtgtattatgtctgacactttgcttggtggccaatagcgaaacacagatctccctatgatgttctttattggaagtggacccctgcgttttacattcaccacatcatccatgaatcaacagagttcccataaccaagacagaacaaccattaataatatattcgaaagtggttaccagttatgagaatcaaagcttttgttgcggttgtctcctaggacgaagacataaccttcagggacaaactgcaaataaacaggaaaaagaaagagatcaagttagcagcattcttctaataatttttaggtcgagaaaacattcaataagcaagcgcaagtttgcttaccattggttccatttcatagtcaattggctctaagacaaaatcctctgcttgaacagtgtcatttactaagagctttccatcacaaacctatatgatacagcatcaataggttagacaggaatataacaacaacagaaacaaaggttaagagtctgttttgaggaggctggtcatcaagaagccactacataagtaataccgataaagatgaccaaagacttcaatggagaataggatcaatcaacaagtggattagagataagggcttacttcaacccagtcaccttcgctagcaactatcctttttatgaaaacatcagcacaactgtaaccatgttcctgcaattcaaaggagagtatcaagcacctttgaaaaaggtctaagaacatttgaaaacccagaaatagtgatttgagatatataccaccaaaataggaggagccttgaagataactatgtctgaaacctctggcttcctgaaaaagtatgagacctgtccaacaccaaagcaaatagacagatttgatcacaatggtttagtgtgattaagccacatcaccaattaaaagatcaatgtgcatttcttatgtaagaagaaacacatttaagaccatttccaataacaaattttagttccaacaatgcagaattggttaaagccgtcattttaaaaacctctcaagatcaaacatcattctacggtcacaattaagaacagaacaaaccagtaatctcaccttctcggctataacacgatcacccacatcgagagtaggaagcatagatgttgaaggtatagactttggctcggccaaagccgatcggaaaaggagagaaacagtaaccgccgtgaaagcagccttagcatcctccgagcagatattcaacagcttattaacccatccgtttccaccattcgagactttatcactcaactccaacttcactttaggatcacagacctttcctcctctatcaacctcagcaatatccgttgataacgtcgccggaatactacaaggcatccacttggaacccctaaggaacgggatgacagaagaagtcttaaagggagagatcccgagaccggtcatacccgaaaactgtggagctccagtcaaattcatgagagagatcatacccaagacaagtggactcttgcagccttcttcgaggatttccctcgcaatggtgctatacattgatgaagctggtcgagcacgagggcctgagcttggaccccatgtgttggatccgggagatttatcaacaatatctggtatctgattgtggccgcagaacctgggacgaacccatgtttcgaagcatgatctaacgtcgccggtaccgacacgagtcccggcggatgaagcaatgctcctagcaacatagctggagtaggtgaaggtaaccctaatcgccataaatgcagattttgaagaaagtaagggtttcttaaggaagacctagaaaggaataaggaattggaagaatcgatctgccggaaagagaaacggagagcttgttccgccggcgagatcgagtaattgtttcaggcggggaagcgaagaagcgaagaagatggagatctcccagaggagaggagaaggttttggtcgaaagcagagagagtcttcttcttcttgttgttggttgagttgagtcatcaattacatttagggccaaaaatgggcctttaaatatacaatttctattcataatatcccataattaaatacgcaagtatcaaaaatggctataacattgacaaaaataaattagaattatctgataaaaagatggtggagtgggtatttcacaaaattacaattgtgaattttgttgtactgagcaaaattgtttaatacatttttagatgtacaattaataaaagcacacgataaatttcatcgtttttttttcaatcttttgctcgtaagaacataattttttaagccgatttgaattttaaagtaaagacattagcacaacctatgtttcttgtcagttgtcacatattaacatttgctaaagatattttggtgtttataaatcaactataagaaaataatatataacctagatatatgaaataatgaatcatgtatagaccattttgatatccctataatacatggtagacatgtagttatgaactagagacgtaacaaggtaaaggctctgggaccgggcgcaggagtgtgtatgtggaaaactataaagaaagtgaatgaccgcagcacatacgtcatctctctcttaaggttcgcccgaagcttcgttgatatttcttaattaatcagtattatctttaaataattagtcgttaacttagttgggttgagtttgaccacaacctaattaacaaaatggattctgtttcgtttttttttcttttagccaaacactaatctctcgttgtcttcccaaatgcttctatttaatcaaatatttatattgtgctcaagatacatcgaggcttcatacggattagaatttgtctatcggcatgttttgttttgttttttcattttttggcatagttttgtgggggcaatttacttgttgaaccctagtttacatgtattctcttgaatttctcacaattttctcaaatttcgatttcatagaaagttagaaacaaacactcacaattcacaaattcccactggcaaaagatgaaaaagcccaaaaaataaaattacgtatcgccatcaaatagggcctattggtttaaatgtggttttattgtatatcagatattaaaacaaaacaattgtcatcaaagcaccagtggtctagtggtagaatagtaccctgccacggtacagacccgggttcgattcccggctggtgcatgagctgtgaagaaattggctttagcattggttgggtccaatgcattctactgaatatcagttgatattgccccattctgagctctctccttttttttttggaagttattttgcgaaatagaccgagcttctccaaggacgtccacagtccagagagtagatgtttcgtctgataacatggtaacaatgttctaaactctccacgttcttattgttatgcatgttctattgttcttgctagctcagtttggaaaggttggaacttttgttggttctctccaacaatgggtctttgcttaaaggttgaatcgtgttagatgtctttcaaaggtcgaatattttcgttaacagtcttacagagtctcaaaaactaatctctcaatgcttcgtctctcatttcaactccgactccaagtttgggaaattttgtctcatggagaagtccggttgggagaagttgtctcaaaactctaatgggatgacggatgtgagggtgtcttggagaagaagctactgaggaactgccttggtgttgtcgtatcaaaatcattgatcccaaaatcaaactattaacccaattggaatgaacttattaaacatctccatctcctagtaaaataaaacaaaacgtagcaaacatcattgtatacaaaagaaaaatgaaatttattagtctcctttgttccttgaagaacaaacaatgggaatagcaaacaacgaaactcacgacacataaccaacacaaatgtatacgtacgtcctttcttgcacaatggcacctcaaaaaaaagaaagaataaataatcaaaagtgaatcaaatgaattgaaaaaaaaaaaaacctattaacagaaaaatgagcctcatcataatcttgtcgtcaataggcagagctactctcactagcagagagagaaggtccttcagatttgactaagcttgagctattgctactaatctccagcccttgcattcggccgttctgtagaaagtccgacacgctttgagaattggacatctccataggattgaaactagaatgcatctgcatatcatggaggttcattgagccatgatatggtggaatagccatgaaagttgaggaagaatactgaatctgctgcattcccatatcaaacgaatctgaattaccagagatttctcctgtctccatcttcatcctttcaacttctttcctcaacgcttcgtttaaagctgtaacatcaaaaagcaaatattgaagaaatgcaccaaaatgcatacaaagaaattagcttcttttttttttgtgcttaccattacgaagctgagcttgttgttccattgcttgcaacctaagtttcagctctgtgttttcgtttgctagtccatttgtgtctctctggatcaatcaaaacaacattgaaatcaagatctgtctatgtatgtaaaacagaggaaagaactttcctattctgttgggatcaaacgaggaatcgccgaatatggaaacctggtagagagtaagctgagcagagagagtggtagcttcggtttgaagagattgaactttgcgctcaagttcttgaatgtatcgagcttttctctctttggatcgagctgcagattgtcgattcgctagaatcctgtaatcaaatccctaatttcattgcaattcttcaattctgtggaaagagaaacaaatttgaagaagaagaagaaggaccttttggcgcgtttgggatcgatgttccaaagctcagagagtttttcaggaggcatagctttcttagcgtccatgatatcaccggcatacatggcgcatccagcgtcaacggaattgctgtgacggtggcgaggacgagaggaaggaggaggaggaggattagcagcgccggaaacggagttggaggagagggaaggattttgaaagggattgggattagaggtgaggctatcgacatcgatgaaagaagagaagagatcgtcgtcggagggaaggtcgtcggaggaaggtggtgcggcggaggagaggggatccatgaacatgaacatggacatatcgtcggagcgagatcggcggtggaaggaagaagatggaatcggatcggcattaggaattggatcggaggaggggataatagtggcgccgggctttgggactggtggaggatctgatttctccatttttttaagagaatctgagagatgaatgctttggttgtgtgaataattatgattattattcgcaattgagaacaatgtcaatggagataatgatggagatgaagaagacgaagctctttctctctctctctctctctctctctctctatcaaatgaaatgaaatgaagagcgtaggagagaaactcgactctgtgaggagagagaagtgacgtcactaactataaaattgcgaaattgagttgatttattaataattaaaaagaaaactagttgatgaaaagtaaaaggcacattggcacgtgaaccatcgcagcatcttgtctccctcccttttttttttatttttttttcctttttctttgtattttatttaattattcgatttcatgatttaaatttgtttaactttaaatcttctttgtagcattcattacaagattctgtagttgcttcaacgaaatttattaatttaagctgttatggcattacatctccccacgtctgagtgtatatttttgtatctttgagtcaaaattttaacaagtaaaaaataggcagaaagccttgtatgaactgagagtctgagagaatgttttgtgggataataagttggtaacttggtatggaactatctatggattgggaaacaagtaaaattcacttatagtgaaaaatacttattggttggatctaaatcatacggaggccttaattatcaggcccatacaagaagacaagtattgttttgttttaagaaatcctcgagcatataatgcgcctgtagtggacaggaggatgttttgggccttctcttaaattggcaattgggcatcactttgtttaccggtcaaaataattaaaaagatggtcgtattgatcgctttaagtaataagttttttagttaagtctctctaaattgtcttaaacgcaagtagtggaccggcatcatttaatagtgattcaaattaaaaacctaatgatgcttacttttaccagctaaagtttcttttgtcttttttttttttgttttattattatttatcaacatacttgtgaattgtgattctccctttttttatatataaataatgaacatacttaagatatagtttctcccatttattgtcacctaacgggtattttttaaaaccaaattctgttggaaagttcttctctaaacataaataatatatagctcatgtctatgctttttaatagtgcccattgtatttcttttcttatttgagtaatttattccagctgtttcttttttttccctctcgtaatgatacgacatagtgacatacatcttctgtttcttctttttttgttcaagaaaatcataatgactacaggtaattaatcacacttctacaaatcagttgataatcagcaccattttccaataaaagtcgatactttggaaaatttcgtgacttttagtatcgtgggggccaaaatgcacataaagatacgatatcgatgaccaaattgccatatactatatgattccaatataccaatgattgctactatttgctaatggtatacgtttttaaattttaattaatacataaaaaaaaaatcaaggtgggataaaaaggtaactttattgacaatatttggaaattcatcgcacctggcaagagatttacctactgtcattaaaaaaggattaaaaaaacgacaaaaaggtcaaagacaggtcagacaaggaatcattaaacaaaggaagaaaaatagcaagtagaatggttttgacggaagaaggtggtgaagttatggcggcggcgcaacggaaacttatgatgacgctcttccgtcggtctcatccattacctgaaatagtcaaacttagaaaatgtcgtattatcactcttcaatgctcccacccaataccaactactttcctctttctttgtcttgtgtttcttacttggtcatggccttttccttctaaactctctctctctcgagatttcgttttttccttgggtctcctctgtttcctcctctctcgatatgttttcgacccatcatgtctctcgtctcctcatacctctgctcttcttcttcctcttctgttgcttttcttctacttttgctcaagacgacatcaccaatcccgttgaaggtttcttttttgttgttgttgttgttaaaaggctttgtctttggtgttagatcttcagacaatttgtgtctgtgacaatgggttttcttagctttcgactgatttggtactgatcattgatgttttttttggttttagtgagggctttgcgagtgatcaaagaaagtttgaacgatcctgttcatagattgagaaattggaagcatggagacccgtgcaattcgaattggactggtgttgtctgcttcaactccactcttgatgatggttatcttcatgtcagcgaattgtattgtcttcttcactcttgcttttttcttttatttggtcttccttgagatttgtttgagttagctttgtttatacttgtaggcaattgttcagtatgaatctctcagggaacttgtctccggagcttggccggttgtctcgtcttactatcctgtacgtgtttttaacacgatatgttgcttgatcttttgtcatactcaatggtgaataactttctttttgtattgtacaacttcttctctaatgccaggagtttcatgtggaataagatcaccggaagtataccaaaggaaattgggaatatcaagtccttagaactcttgttagtccaggactttgttgtttacttccctgctttatttgctttgggattggctgctaacttcacttttccaggctcctgaatggaaatctgttaaatggaaacttaccagaggagctagggtttcttccaaacttggacagaatacagattgatgaaaaccgtatatcaggaccacttcccaaatcttttgcaaacttaaacaaaacgaagcacttgtgagttctttaatctggtgtcgagttgtacttggctgtttcaatatggtgattccttctccccttgaatatgttacatttattgcttttttttggtcaatcttatctttttccagtcacatgaacaataattctattagcgggcagataccaccagagcttggaagcctaccatccattgttcacatgtgagttggtagcagtagcaggcttaaatgattttacttgaaaaatgctatgttttttacctaaactcagttttcatgtctagaacatgctacacacgctttacctattcaatgtttatgaacatttacttttttttttcttttttcttacagccttcttgataacaacaatttatcaggctatcttccccctgagttatcaaacatgccgcgtttacttatcctgtaagtttatgtcacagtttctgttgatttccctggtcctgtcaccataaggactggttacccatgtgtttcttcttttcttttttttgacctttttcctcatgtttattttggcagacaattagataacaatcactttgacgggactacgattccacaatcttatggaaacatgtctaaacttttgaagatgtaagtgatagtttcattatgtatctgaaatcactgatgtgtgaggctggaagggctgtcatcttctattaatgcattcaacaattcaatacgaaagtcatgcattggatttgaattaacctgtgggaatagtaatcagccatatttgagctaattaggatagtgcattctaaatgtagagatactggttgtcgttttctattttttcctgatctttttgggcataagatacatgtgtatgctatgctaacttatgcatatttgtcgtatgcccacgggcaggagtcttaggaactgcagcttgcaagggccagtgcctgatcttagcagcataccgaaccttggttatttgtaagtcttataacttatctagacgcttccttatatagtcgtgcaagccttagcgaaggcgaattaacctcatctatttctggttgtcggacatgaattccagggacctaagtcaaaatcagttaaatggatctatacctgcagggaagctttctgatagtatcacaaccatgtaaacttttagcgcttttcataattctggcctttgatttaatccgggcaatgtactgggattagtattctctttttggggttggaaatgaagtgcttttaactttgatatcttctgatgaaaatactgcagcgatctatccaataacagtctaactggaaccattccaacaaatttctcaggccttccacggcttcaaaagctgtaagtctgtttgattggtgtagcgagttttgtttggtttctgaaatgctgttgttcgttctatcttggatatgaagatggctgattgtttgttttgttgtctacctaatgtgacaggtcgcttgcaaacaatgctctgagtggttccattccctctagaatatggcaggaaagagagctgaattcaaccgagagtattatcgtgtatgactgttacaaaaacaatcatatctcctatcctgttacctagaagcatgttccaactttctttatattgtctcttgacaaacgtcagttcctttatgttttcagggatctgcggaacaatgggttttcaaatatctctggcagatccgatctccgtccaaatgtgaccgtctggtttgcttctgctctttcctatttctatttattatctgcccatacgtacttatttttgctttgcatatcattctagctactcaatgatgtatataactgctttttggatcttgtttgctcattctgaaatgtattaaactcttttcctcagattagtgtccacttttcgggtatcatttaacaacgtttgttttttcaggcttcaggggaatccgttgtgctcagatggaaatctgcttcgattgtgtggacctataactgaggaagacattaatcagggttcaaccaattctaatactacaatttgttctgactgcccacccccttatgaattttcaccagaacctcttagacgttgcttttgtgctgctcctctgcttgttggatatcggttgaaaagtcctggtttctcggactttgttccttacagatccgaatttgagcaatatatcacctctggtcttagtttgaatctgtatcagctacgccttgactcattccagtggcagaaaggacctagacttcgaatgtatttgaagttctttcctgtttttggttcaaatgccaacaattctttcatattcaatcgtagcgaggttcggcgaataaggggcatgttcactggatggaatatccgagacgaagatctctttggtccttatgagcttatgaatttcacattgttagatgtctacagagatggttagtggcagaatattcgtcctctcatatttattcaaaacagaacgtggttaaaatacaagtaaacgagctgatgtctttctgctacattaaacataaaagtgtcatttgtttatcagcgtccctgataacaatagatcctgtcttacctttttgttcttacgaagctataaaacttttgcatacatggacattgttttgactgtcatgctttttatgttcagtgtttccttcagcttcgccatctggtctaagtaatggtgcagttgcgggaatagttcttggttctgttgcagctgcagtaacgctaactgctatcattgcccttatcattatgagaaaacgtatgagaggatacagtgcagttgctagaagaaagcgatgtaagcattttcttgtggatgccgatttgtgctcgagtaaatgatttatcggaattgtacgttttttttggtttttggtgttacaagtttgattcattcatgtgcagcttccaaagcttctttgaaaatcgaaggtgtgaagagcttcacttatgctgagttggctctggctacagacaattttaatagttccactcaaattgggcaagggggttatggaaaggtatacaaaggtacacttgggagtggaacggttgtggcaattaaaagagcacaagagggatcattgcagggtgagaaggagttcctaactgaaattgaattgttatcgagattgcatcacagaaaccttgtttcgttgcttggattctgtgatgaagaaggcgaacaggtactttgtccaaattcctcttctggttgatgtccaaaacgtttatatgtatctcttattgcttgtattgttaagcgtgcagatgctggtttatgagtacatggaaaatggtactttgcgagacaacatttctggtatgttttacttcttcactgagtcatctatagtggtgcttgttctctatgtgaatcagcaaagactacccaccgatgttcactgttcttaatgtgaattctttggagatgacttataagcagcatagatgatattgtgtgcttttaagttcggctctattcctcgtgaataacaatttacatacaaatatgtgttttggcagttaaattaaaagagcctctagactttgcgatgagactacggattgctttaggttcagccaagggaatcttgtatttacacacggaagctaatcccccgatatttcatcgcgatatcaaagcaagcaacatattgttggactccagattcaccgcaaaggttgcagattttggactctcaagacttgccccagtacctgatatggaaggcatctcacctcagcacgtgtctactgttgtaaaagggactcctgtaagatctcattcctgcattgcttctctaaagtttttttctcaaactcatgactgagagttatttgtcgacttgcagggttaccttgacccggaatatttcttgactcatcaattgacggacaaaagtgatgtatatagtctaggcgtagtgttgttagagctctttactggaatgcagccaatcacacatggcaagaacattgtgcgagaggtaattctgagtagataataagagcgtgtgttttccttttaggcgttatataaattgtgtggaatgtttgtttctgtcagatcaacattgcctacgagtctggttcgatattatcaaccgtggataagagaatgagctcagttccagacgaatgcctcgaaaagtttgcaactttagcgctgcgatgctgcagagaggagacagatgcgaggccttcaatggcagaagttgtaagagaactagaaatcatatgggaactgatgccggaatctcatgtagccaagacagcggatctctctgagacaatgactcatccatcatcgtcatcgaattcttcaatcatgaagcatcattatacatcgatggatgtctccggctctgacctcgtcagtggagttgctccctcagttgcacctagatagaaactttcttcgactcatacaccatacactgtttctttgattattcagtgtgattttgttgttgttctttctttgtacaggaaaaatgattgtaagcgatgtaacggtatatgttgcagacattttaatatgtctgcctttgtgagttatctgttccgcttcttatgtgttttgacttggatcatactggatttttttaccttcgaatcaatataacagtcttataattatacgtacactttcccaatgttatacaaccaataataataccaaaaaacacattacaaagatttggctcaattttgttccttattgaataacatatacatagctaattaatatcataaatactccttcgttgttgtttaacataaatatggcttcactaggggaacgagcgagctgcttaggtgcatggacattacttcgttggtcgaaccaacgagcttgccaccaatgaagacggctgggaccggctttgaacaccctagcctcataagagccttctctatctctcggcattcagggtccttatcaatctcgtggatcttagggttaacaccaagatcttggaagagaacttgaaccgcataggacaaacaacaggagctcttggtaaatataaccacccctttttcggacgacattctcataactttgtccattatcttgcataaggtgaagaagaagaagtatatggattgaaaggagtagctagagtctcaggctatttataacaggagggaagaataactgaataagaatccacatacagagatcacaagaagatcgttgaaaatgttcccaagtccgactttattatatatgagcaagatgattcgacgagaccgaaccttttcttcggcagcctttacccttttgattgtaaggaccaataatatgaatgatcattccatatattttctcttggatacaaagatgcggtctcaacgatcctcctacaagttgctaaaaagagagtgtattcgacgttgaatatagatagatagatgcttcatacttttggaccatttgatttattctgcagattccatgaatatcggacataaggaatcttggtgacccaaactatgcaacaagaaaatcaacttgcattcaacttttcatagctattccaacttggttgggtctcttaaaggcaaaagagctcacatatttttgtatccttaacaaatcttgccgtaatgttgtttcttttgttcagattatggttttaatgtttaatcaaagcttgtacaagtgtaacgtagtatacacataacgtaatcatgatcaaagaagggcaagaatcatatgagaccaaaaaaataatgacaagccaaggcaatgggaaaggaaaacaaattcagaaaactgaaatgaagatgaagaagagcagatgatggagaagagaatcagattgaccataacagtggatcgagcttgtcagtcgtttttcgtgccatatagatatgatgagaatcaaagaggggtatacaacgtgatgattttgaagggtatagttgtcattcttcttactgtagattctgattgaggggtaggtaatttgatttcgctgtagaaattgattggttttgggaaatattcaatccaaggatagtgtcgagcaaccatggatttgtgttttcaaaatcccgtaaagtgtggtgatcgtttgttctccgcattgaatacctctacgtattacaagctgggtacttcgaatttagggtttaatggtccagttttagagaatcggaagaagaagaagaagctgcctcgtatggttacagtgaaatccgtttcttcaagtgtggttgctagtaccgtacaagggacgaagagagatggaggagagagtctgtacgacgccattgttatcggttctgggattggtggattagttgctgcgactcagctagctgtgaaagaagctagggttttagttcttgagaagtatttgattcctggtggcagctctggtttttacgagagagatggatatacttttgatgttggctcttctgtcatgtttggtttcagcgataaggtttgtttgttcatcatcttgtgattttcaggagaactgaacattagtagaagaagcactagtttgtttggtccttggctaaagtaataatggcttaatttgaagggaacaggggaacctaaacttgataactcaggcattgaaggcagttggtcgtaagatggaggttatacctgatcctaccactgtccatttccatctccccaataatctttctgttcggattcatagggaatacgatgatttcatcgctgagcttacgagcaagtttcctcacgaaaaggaagggatccttggattctatggtgactgttggaaggtctcatgtgatttaatttgacttttacgagtgttcactgatatatattacaacttcttaactgtggtttgaacagatcttcaactcattgaactccctggaactaaagtcactagaagagcctatctacctttttggacagttctttcagaagccgcttgaatgcttgacactcggtattcatctaaatcttctgaaatgaatttggacttaaagttgtgtttggtgtgtgtgtagatatttattgctgtgagtccgtatacgtcctgcgtagatacacatagaaaaatctactgacatgaatcttgttgtgttattctctgtgctgtatagcttattacttgccccaaaatgctggggctatagctcggaagtacataaaggatcctcaattattgtctttcattgatgcagaggtgagaaaaaaacattattttttcttaacttgcacaatttttttcactcaaatttatatccagaatatagcatggatatcacccttcatatttgcctgcagtgtttcattgtgagcacagtcaatgctttgcagacgccaatgatcaatgcaagtatggtaggattcttgtttttgcatttggtgtctactcgtgttaccctttattttattttttcttatgtcttcaaggacaggttttatgtgacaggcactatggaggaattaactacccggttggtggagttggtgggattgccaagtctttggcagaagggcttgttgatcaaggcagtgaaatacaatacaaggctaatgtaaaaagtataatacttgatcatggaaaggcagtaagtttctgtatgagtcttgttgaagttttatttttttcattaaccctttgaaattcctttttggttatgaaaatcgttttattttctggaccttaattttcaggttggtgtaaggctagcagatggaagagaatttttcgctaagacgataatttcaaatgctacaagatgggatacgtttggtaagagaaaacaatgacttatgtaactgtcgaatagcttcctaaagaaaaagaatattttacaaatcttgttttgtaagtgtacttaaaacttgtattttgctgtgctttagggaagctgttaaaaggagaaaagcttcctaaagaagaagaaaatttccagaaagtgtacgtcaaggctccatcatttctttcaattcacatgggtgttaaagcagaggttctccctccagatacagattgccatcattttgtgctcgaggtttgatagtttcctactaagatctattcacttgccaggtttatagttattgaatagcgactttcgttttgctgcatccgtaggacgattggaagaatctggaggagccttatggcagtattttcctcagcattccaaccattcttgattcatctttggctccagatggtcgacatatacttcacatatttacaacttcgtccattgaggattgggaggtaaggctactaatattattgctattttccttagctgtagaattatcctattgagtttttattgatttgctcatctttgtttatcaattcgtctatattttggtactagggactccctccaaaagagtatgaggcgaaaaaggaagatgtggcagctagaataatccagagactagagaaaaaattgtttccagggctcagttcatctattacgttcaaggaggttaggcttgttctttattcccctactattttacatgcacattgtagaaatggtgatagtaggtctgcatgatatgagaaatctgtatctgaaacccatgagaataagcctatgccttatttgggtatttagcctgtatatctaaacgttttttgaggcaggtgggaacaccaagaacacacagacgatttcttgcgagggataagggaacttatgggccaatgcctagaggaaccccgaaaggtttattaggcatgccattcaatacaacggtaagctaaagaaagagatagatagatagataacatcaggtttagttagttctggtggtgaatggttttgctaatgaaagaatggttggactaataataggctatagatggtctatactgcgtgggggatagttgttttcctggccagggagttatagctgtggctttttcaggagtgatgtgtgctcatcgggtagctgctgatattggtgagaaaatgccatatttttgcaatatgttaaatagaatgggttgattattatgagcataagaagcaatcacttttgtgttttgatttttagggcttgagaagaaatcacgagtacttgatgttggccttcttggtttacttggttggctaaggacactcgcatagatccaatacattatttccttttcttctttattatgttaatttctattgttttactatttcttcatagaattttacttttctttattttcttatttcctcgtttacttttttctagttctatataaagaagaccttcttgtaagaaatacatctcataataattttcttaagaagcatagagtttttctctcttctactggattatagagtttctggtggattccagaattttatttttcaaattcatagcttgtagacttgtaaaccgatataggtttggcgcttgcattttttagggtttttcgaccgcgtcagatttgtgcatgggaaaggttgattcataaaaactgtaagcccattaaagtaaagcccattgttaactctaaattatcttcattcagagttatatcttcattcattcaggaatttcgaaaccctagtcaattatcgagatcgatccaaccaatttttttttaaggttaaaagtttttgattcgtgaatgatgatggagcctccgcctacgaaggagattgctttggcccctgtatatgtgtattgggacatgaagaggtgtccggtccagatgactatgatgctcgtcgggttggtccgtgtatgaaacggattttaaggaaattaggctacaatggtcctgtcaccatcactgctgttggctcactagcaaaggtccctcgtgacatccttgaagcggtttcttccactggaatctctctttatcacgagttctacagtaagcatttctcatccttattccctttttattccctttattagataaagagagtctctctaagtatatagtttctgaaacaggtcgtaaaagcatggtttcgtgtttccttggccatggtatattgaatccacgtccatctactatgatggtcatatcccgtccaccagtgtacattcctccacgcttctattctaatattagccgccgacgtgaaaatagatacaactccattttcccgtttccacttgagtctcctcgagaagcctcctccaccctctggaaaaagtttcttttagctgatccagggccacttgatgaggaggatagctgcagtgaaacgcctggacttgcctcctggctttgctctgtgtgccgtcgtattggttgtggtccaggtattgctggtcaaggcgttgataattttatcacgcatatctctactcgagaacatgaactcaatgtaagctaagcatccttgccactagtttaactatattccctttctcagttgttcttttctttgcctacatttgaccctgtgttgtctttttcctcgcagcgtcgaggctgtattaccccaaaaggtgatcgcaataactccagattgaaagagagagatatgtgtaaagaagaacataagaaaatgatataatgagaacaattctttttaatccgtaatgtgtgtgactgccaaatattctcaaatgatccaccattttcatgattttttggctgttagaaataaaacgtttagaatttgtaccaaaatacggggatggatttaaacaatcaattgtacgttaagttcaacacatttgaaaatgatttatacgcatgcatcatgagcattattgtattattgtttattttatataggtccctttatatattataaaagagtgcaataaatttggttacgactttgaaactcatcgagctttcaacaatggcgaccattccctcccacaaccttcgtcctcatacgaccaaccaaaggactcagtactctctttccttcagaccacacttttcacgctctactctgatcactttcccggcaagatcatcgccggcgagggctatgtccagaaccgacgaggaggcttcaatatctacaaggctcgagcaagaaagctatgggctgacgacggcagaggacattcgccgacgggatggagaagcaaaagaatccaagaggttgagagacacgtggcggaagatccaaggggaagatgattgggccgggttaatggatccgatggacccggttctgagatctgagctgatccggtacggagaaatggcccaggcctgttacgacgccttcgatttcgatccattttcaaggtactgcgggagctgcagattcacgcgccgtcacttgttcgattcgctcgggataatcgattctggctatgaggtggcgcgttatctctacgcgacgtcgaacatcaaccttcctaatttcttctccaaatcgagatggtccaaggtgtggagcaagaacgccaattggatgggttacgtggctgtatccgacgacaacgaagccacgcgctgccgtttaggacgccgcgacattgccatcgcctggagagggactgtcacgcggctcgagtggatagctgatctcaaggatttcctcaaaccggtatccggaaacggattccgatgccccgacccggccgtaaaagccgaatccgggtttctggatctatacacggacaaagatacatcctgcaatttctccaaattctcggcgcgagagcaggttcttacggaagtgaagcggctggtggaaaggtacggcgacgaggaaggagaagaactgagcatcaccgtgacgggacacagtctcggcggtgcgctggcggtgctaagcgcgtacgacgtggcggagatgggtgtgaacagaacgaggaaaggaaaagtgattccggtgacggcgttcacgtacggaggaccgcgagttgggaacattcgattcaaggagaggattgagaaattgggagtgaaggtgttgagagtggtgaacgagcacgacgtcgtggcgaaatcgccgggactgtttctgaacgagcgtgcgccacaggcgctaatgaaattggcgggaggattgccgtggtgctatagccacgtgggagaaatgctgccgttggatcatcagaagtctccgttccttaagcccaccgttgatctttctacggctcataacttggaagctctcctccatctccttgacgggtcagtcattcttctttttattacttttttactttaaaaagtaattttaagatttccagctaatattttgacaagtcaaaagcctttgaggagacagatctcaatgtccacattttccaacaccatttctttattaatacgattttcaaaaatttggtaaacattattgcctcatcaaatgccgaatggagaataatggggtcatttagtaatctgatttacgtggcacttctttctttcatgggtaggttggagagaatttaattatcgaaaactactaggaaataaatttgtcgtttagagcataatagtattagtatgagttgatgaatctggattattgtttaaaaaaacataggtatcatgggaaaggacagagatttgtgttatcaagtgggagagatccagcgttagtgaacaaagcatctgattttttgaaagaccatttcatggtccctccttattggcgccaagacgcaaacaaagggatggtgaggaacactgacggccgttggattcaacctgatcgcatccgtgcagatgatcaacatgctcctgacatacatcaactcctcacccaactccatcatccatcacaactcttgtaatttatcgatttttttttggtttttaatcttcctaatttgtgtcattgcctttttacattacagataaacaaaattgaacgtccctatcttagtgaaaacccgtgcatagtttaaaataaattttaagttttcatcaaaactggcttgatcatcatctgttaaaatttcatctgacaatgcctagactaagtctcggtctctcacccttcttgatcaacctgttcataagaatcccacctgtttcggtcactcactcttcttgatcaacctgttcataagaatcccaccaagaaacaggacccaaaccatcatcatccatattcccctgcgcagtacacaagattgattgaccactggaccgacgtaggaaaaccaaaacaaaaaatcatggtgtctcaaaataaaagttaagtatctataagagaggagggaatcaggaggcttatctgaccttacttaactagcgttgtttataatgcatatacaagtggaaaagtttcagagaatgagtttgatgaatgaacttacagtgaccaccattggagcaaaaggctttgcgtcctctgctcgctcagttggaactgatggatagctgatgctctctactcgaaacttgatctgccatttacaatgcctcgttataaaaacataccacattcagataattatgcttaaagatacatgcaggaaccaaaaattcacctgacaggcatcgtcaacaatatagtcttctttcggttctccatattcccacacccatatcatttgcttcctgtgaaccaaaaaacattatactttgtgatcaaatgatatataaagatgttggtgcaatctcctactcatagttggagagctggtagtactacataaactgcaggacacatattcttacctattataagggtcaggctcacagcggttgggtttcggcataagtggagcaggcacataaatatcgtcaaagaatccaagtgttactgccaagtatagaggtaccgtaagcatgtaaatgattaaccatatccatgatttcaaagtattaatccaattcataagtagctaataagctaacaatgtactagagtagcaggatgcagacagtgcacaacattggaattcttcagggaacagtataataaagtactcacagcgcaagccattggcgtcagattctttgaactttgcagctatgacctcaccaacaaatggacggaacacaacaattctaagacctacctacaatcacacaaacaaagtattaacaacacgacatgagatattgaatcaaggcaagataaaacttttgaaaataaaattcttaaccaaaaccgtacagtgaaagttagactaacagataacgattccgaagaaccaaaatctcagaatggtttgaaactctagtacggtaatgatgagtctacaaagatgtaactcaatacagtgcagaaagtgtgcaatacacggtgataggtcaacaatgatgccactaacagtgacattgggaggccataaaattgatataaaaaaccatgttcaactgataaaagaatagagatgagaaaagatcagcaatttggatataccttataggtcgcagcaccatcaccgggtaacacaaaccctccttctactgatttaatgtcgtaaatagatacacaaagccccaaatcagctaaaacctttcatagcaaaaatccactttgataaaattagccaggtgtgtatataaataagcaaacaatggataccataaaagtgaaagaaaaagggcaatgagcaaaccttgtccaagaacacattttgaagcactgacttaatggcatcttcgagagggagattaagtagatgcggaggcactcttagtgaatgttctagctcgctaagataaaacatttctgaaaattttgttcaaaattcacaagtcagatgcatggaaaattggaaacaaacaatcaaagaagtagaattcgaataacgaatcagacaaattcggagcattcttggtcattaacatgcatatcatcaaaagttccaaaattcaaatctaaaattcattaaatcatagtacgtaataatagcccaatcctcaagattaagcatgtgcagagcgtacaaaaatctgccattgtcataactatacgagagaacaaaactatctaaaatcgggcaatacctaaaccattgaagataagatggtctcgaagggaactcggagagatggtgatgaagacaacgattagacggaaaagtgagaagttgggaatagattggaagaagagagaacctactaatcgagtgaggaagatgcggaagaagaagaacgatttagggtttagttattaaagtcaagaattaaaacaaaccgatcggtttgtcatcgttacgcttataaaccgaaccgaactgaaaaccgcactctttttattcataccggtgggccaaaaaaggaaattgggccaagtccaaggtatgacaacaatctgttgctgtcagcctgtcactatagtctaagtaatattaagctctgatgaaaagtgtgaaaaaagaagaaccaagatatacactgatgtgggataaagaatgaagctaatcataacattgatatcatgcttgagtagcttgtttaacaaaaagaatctgaaactgaacctaagttgccagattttttttttttttttttcgtatcaagcttgaagattgcactgttgcaagtaggtgtgatggtaagggacgtgtatgttccagtaacgtttgtaattctcttttccaatgtccaaccacggcttcatgaccccatcgtagtgtataactgccgcttgttcgatgtcaaccgctttgactcctgattcgcgacctaaccccatcacatgccatctcttgtccaatgctaatgtttgcctatagaaagtcaaccaacctattggtaagctcccagctttccacaatggtctctttgttccctgccaaaaaaacaaattccccaaaaccattgttaggtttacgcccattacatagacaagatatgttgttgcttgtaaccattcttcttctaggagatgatctttagcactagcttgcttatgttctttgctagttacagcaatgttatttgtgttctaaattcaaaacctttcatcctcgatatagccaacctaaaacctaataattattacaatctttttcatatataatgacgaatcaacaaatctaagaacccatttgaaaactttgaatatggctacagtgttatttccatagggagagcaagtgcagtagagtgtgtgtgtgtatatagcgtaccaggttgaagtattttatgtatgtagaagtcaacttccgtattctccattcttcgagatcaattagattcatcccgaaagcccatgtgcaagctctaggactaaatttcccagcgacccatgtgtctgagaaattaataaatgtgctcattgatcgaaatgaagattcaccttcaagacaagtctctacagctccaaccacctttcctttcatatcaatgctccacagtctacttaaatctctttgaacaactacatcatggtccaagagtaccatcttgttcaaacccgggaatatatccgggagatagaagcgtgcgtgattgagtgtagaaatgaatcttgggtcattagagttttgcttcatcagtaattgatcataatctctaggcaggacatccatatcatcaatgtttaggatttggatagtagctttactttgaatgtttagcagaaaccacattgagattgctgggtaattaagtgaatcagtcacgacatggaagactattctttctggctcctgcaaacagcaatcagaagccacattaataagtggcggaagtaaatgagaagatatagtgtaagacactctaaaattaggtataacatataatgataaccaagtagaaatttgtataagaaatgcatgatacctttgatgaagatatcgtagagttaacaacgactgaagaagccaaaacattgtcagagaagacaacataatgattgaaattagcgtcaaaataattttgctggttaggcatctgccttttttcaggatccagtgaaaagtattctgatgtcagccgcattgagagacagtgaagcccttttggggtggtccttgctgcaagctgcattagatacgctgcttgatttttctgcgcctgaacttgttcttctgtgttataattcatggcacggagtttggtagcgatggcagggcaattgttaaagacacgactagccttatataacacattttccatgggcttcaccctgcggagagcgctgaaagaaacaatcatgtcagaggttactaaaataatattcttaaacagcacgacttttggcactaaaaggttgtagttgtaacagcaaatttctgaatgcaattatggtatgttctccttttgaatcaagcagttgtgcttacccctttgataagtccttgtcctttgttgcatcaccaacagaccgttccagctctttcagccgacctctcaactccttcacaacttgagagttacttccaggtggagcgaaattcaggtaggctttagcttgaataattttgtctctgatctccttcgttttcacatctgttgctctgtcgggctgaacccttgtgttcttttcacccttagataaagggggcttgaaatccgttttattagcaagttggttgactgttggtagaatttgaccctgacacaaaaaaaacccaataaattgtaatcatgaaacatctaataatttgatggaaagtgaaacttggcttttctattccttagataaactaatcaacatggaccgcaatgacttgtaatctgattctaggattcaatatcggagttattccataccctgagcaatcatcaagttcatgttactttatatcagctggactgatcaagttcatgttatcttcaaatcagttgtatttatatatacacaccttttcatcagagctaactgtcatcttctgtgaaacaatcacttgttcttccctgtttttgtaagtattacctacaacccatgattacgacagataaaaaatctgaggagttaatgacatcagagcaaatcaaagcaagattgaaaatatgatcccaccaccatcactttcagcagacgaattaaactccccatccttgaagagaatgagccttggccccttcaagccttctccatcctcctaaatcacagagagaaaaaacttcaacaaaacgcatggtcaaaaggttttaaggagatgacctagaggaacctgtaacttccaacttacatgttcaatagcgctaagtctaaggtcatttgtcgtgaatctctgcagaataatggtagcatgtcaacaacagtgtattgctcgagaaatataaagcataaagagaatgataaaacaagactgagatctgaaaacttggcacaaaatcaacagcatgctgaaaacagatgaatccagagattgaagactaaggaacacatacaattttggataactcttcaataaattctctacgacctgcaagaagacaatcggcaatggaaaatcaaaacaaatgtcagatgaaaaaatcccaagaccaagacaacttctaacaaaagcaagcgaatttcatgtgaatcaatctaaaatcaaagcaatcaagcatttccacacgatcgtcaagtacaatgaaatgaaaataagaaggagaggagacgaaactgaccaacgggagtgatgctcttaagccgattcgatacgaaaataagcggagcgaatacagatatcgatagcagagcgaggatcaaaatcctctgccatcgacgaatttgtttcatgaatctacaaataaaaaaaaatccccaaatctatcttcctcgatgactattcttatcacatcaaaccctgaatcgttctccaggaagaagtagcaagagactcacaaatctcggagatatcctgcttgatcaacaagcaaaagaacgaaacgtgaagaacttagggtttctgggattgcttttctctagtgggagattctattcctcgcttgaatttctggatttggggtcggagagaggagttttagattcagagcttcttcatgcatcatcgggggattgtgctcacttatatatatttaattaacagaaataatcattgtttaatttgtgttaggtctctataaaattaaattaatgttattggtcgaacgaattaatttgtttataactttatagtatatagttttttgggtgttcgtgttgacatgtcaaatgttattagggtgaaaggaagcagtgatgcggatcaggtgcgggcaggtgtacatgtgaagacatgtgattatgccgttgatgaagctgtaagtaatttaatgtgatcagtcttctttgcaaattttatcaatcaccagtcagacgaagacacgtggataaagtttttcctccctcctcttagaagacaagaaaggaacttcagtctcatatcaatggatgattagcctattattatgaaaaagatatctagcatgtcggccaaaaaaaattatggcataaatagaattgtaacgaaaaatgacttttgccgattctgttcattatttttcaacaaaaatgttaactggttaacgaaaaatgtttaactaagtcaaatgggtgttaacgttgactatggatgcttttttttttttgagccaagtaagattacattttgggttttttctttatttaatgaagttgttggcaatttgcttaattcgctattggaacgacgggtcttttcatttattatgtgtaaaatggtggaggtagattatggagtacataacactcggtttaataaatcatgacagtatcatataaagatacttaatatgcgcaaagctttgaatgatcaaaatcgctagctagtgtatcatcaaattgttgccgaaaatcttaactttgacgtcggttaaaacactaatcagcataggacacataaaactacatacgtacatatgaaaatttctctgtttttcctaatcaccattcatctgtatgctactaaaaatcgaggtgtcaatattgaaagtttgaaaccttattagccttcatgcttcgattagcgaatgggtacataagccttctacaggccctttggaacaaaacctcttatctgatgttatctcgtaccccattatacgaatgacgaatcgttacatttggtcgttgattcgaatgttccatattcattgattaatgtaaacgaaatccattattatagttatacaaatcatttttaccatcatctgattataatatgaagagttcaccatcatttacttttcaaaatattaggatagcaaataacaatttctcacccaaaaagttgaaaagaaatttatgtcgttcaactttttttttttacaaaactcttctttcctaattagattaaccaaaataaagaatgtttgacctagttttcctaattaaagtaacacaaagattttgacccaaaaaaaaacgtctttgagcgctcgttttcttgtcgacgagagagctcttaagacagaacacaaagataaaaagagaaacccttgttccaataaaataaaaagaagtgacacatcgcgtctcttaaattttaatagacgacgacggccacttcttgtccgaatcgtacgtcaattcttcttccatctctctcgcaccataacatttattcttcagatcaccagcgaccgagcgaaagaaacgacacttcttcgccgttttcgtccacatgcagttttggggatagcacgttgtgagtgattaagcccaacagataaagagaaagatgatgattaaggtgaagaaggagacgatgagggcatgcttaagctgctctatctgcgacaatatcctccgtgacgccaccactatctccgagtgcctccacacatgtcagttcctctttttaacccaccaaatgtttcgaaattcgctgacattggctttgatttcttctcaatttggatgattatattatagctctgtcttcttcgtctcgtcctataatcgatacaattgaaaagtgggtcaatgaatagtttttgttgaccaaaaagaatatgcaattttgactgcttcaatctctccttcttcgtctttctagtgatagttgattcagcattttcatgtatagtttccttgtgggtttcagcttcttacttgtatgcaattcaatttgagttttttcttataccaagtatgaaatttgctcacatttattataagtcatgttgttagttgctatctactggaatggaataactttttagattactttcgtacgcagtttgtaggaaatgcatctatgagaagatcacggaggatgagatagagacttgtcccgtatgcaatatcgacctcggtagcaccccattggagaagctgaggtaagctattcgtgttctgtatttcagactctaccagttctcatcaaaatatggtttagtttggttaaatttctttgctccttgttcatctaatttgtggactcaatccaattgcttttcttgttggtgaaatgaactgctataacacaatctctggaatcttgttcatttaatacacgtagcacaatggtttttatgagagagagaccatttataggatgttgtaggagaatctaaatagatctcttttgtactttcctttttcttactcgttattgtactgatgggagccacttgtgtgcatggttttaggcctgaccacaatttgcaagacttgagggccaaaatctttgctctaaaacgaagaaaagtgaaagctcctggaattgtgtccttaccaggaaagaggaaggagagatctatatcttctttggttgtgagcacacctatggtgtcagcacaagctggaacaacgagaagaagaactaaagcacctacaagaaaagagttacgtaatggttctttggctgagagaactgtgaagaaggaagaatcttctggggatgagcttctggagagcacaagctcacccgacactcttaacaaattcactcagaataaaagacagtcaaagaaggtgatcactgaactactctattttctcgtaatgacttcatatttgacatgatttggttacatatttatttttgtttggtacacttcaaaagtaaatattcagtaaaaatatttgatactgttattggtctcacattcatttgagttattttgttttttgcagtcatgtaaagagtccatctccaataaagaaaacaaggatggtgatgaaccctgggattccaaaatggattggaaacctttgaactttctggttgaagtggcaaatgggacaaagcccttgaagtcttctgcttctcaagggtcaggttcaaaatctgagcacgcgaatgtatctcgcaatcaatttcaagggagtaaaaccaaaactaagaataaaaagagaaaatgtaaacgtgaggatgacaagagtaataatggtgatcctacaacatcagaaactgttactcctaaaagaatgcgtacaactcaacgcaaaaggtctgccaccactttaggcgattcaaggaatttgccacaaccagatgagtcaagtgcaaaacaggagaggagaaatggtcccgtttggttctcacttgtggcgtccaatgatcagtgagatttttgctctcatatctgaaactatttcctttcatgatctttctctctttcttcattttgtttctcttctcttggtatattgacaagggcttactggttacagggaaggaggaacttctttacctcaaattccagcaaattttttgagaataaggtaggttacagtttaggtattctttgttttatatatcctgtgttgtcgctgcatcttacatagtccctcatttttctgttatgaggaattcttttgttggggaagataatccaatgtctcattctgtgatagggatggaaatacaacagtttctttcatccaaaagtacctcatgaggaagctagatctcgagagtgaaaatgaggtaagggtctttatatgcactatggcacgagaaaggatagaacgtttgaatataatggaacttatatgtttggttgataaaaaatggacatacttatggtttatgttcagatagagataaagtgtatgggagaagcggtgatcccaacgctgacgctttacaatttagtggatctatggctccagaaaagttcaaaccaccaacggtttgctgcattggtaggttcttctgcaaaggattttacgatggttcttgtctatgctcggaagctaccagagtgcaacatgtaaaggactttaaagactcatgttgttgcagaagaagattctgtggagacagggtaatcttttcttttcggtttatggaatctctcaaagcattacaagagagaacattactagaactacaggagcagtcgaatgtgttgtgaaattagtattaatatttttttctatagacgaaatatacactcatgagtcgcttttctgtgcaagaaaatacaaaatttgtttcccttttctttcttcttcttctactatgtgtcctctctggacttgcatgtaacattgttgtcactttctaatcaaatgttgtagtttaagatgttttagattgatttatatgcatttatttgggtactacatcatttctatggaaactaaggttttcaagaattactgttcaagtgttgactgtggatgacgaatgtagtagaactgtgatgacctatatggaggtagaaaactaccaaattaactacataaaacttatctgacaaaagacgaatgaagttcattatcactattttgtgttatcgtgagtaccactatgtggtgacaaccatcatgcttaatatgagtaacatatttatataacaaattgttatgatgttcaaaaaatgttacgagtatgcaatttaacagattctttttaattcatgctttacatatagccatgatgcttaacgatatccaaaagaaaacaaaaaaaaatcattgaagagattgcacctagactagattgcctctcggatgaccttgtacatgtgttgctcttaagaaacgtaatctatgcatcataactaaaggaaagttatacatgtgttttaatatcaattaaaattggttgattacatacttaaactagtaaatcacaatctatgaataatccggactctttttaacatagacacatgacacatcaacaagtagtgtgcaacatattctagaggatggtcatcttaggtagtattcattcaaatgattttggatcaaaaagtaattgaaatcccacgtttattggttcggttcactccttgattagtcaagtttcatattaacataattttcatcgaaattctctttagcattattcgaaaaaccaaatatactgtaatactagcaagaaaaacaatagttttgacatatcttgcttcataaccgaatatctcattacaaaataatccatttttcacgtttagataatcaaacgaaccctcacagaattttctacacaatgcaaaaaacaaaaacgaatttcatcgagtatatcacgagttatggtcaaatgaatcgagtctaaaagcacaacaaatatgaccaaagcatctaatccacatccactaacttactcatcccaccatctacactccttctttctctgccgtcgataaaatttgatccaacgattaatattcaaacctgagaaaagagtagcgatccgcgtagtcattgttttaaccaatcagaagccagatatgaattgccgtctaatacacatctaactatataaatcggacgtttccaatgatcctcttcacaatcctcataatcactttcgaaattacatttacgctttcttgcaatcaaattttccgatcttaagttcagaagacgatgtcagaggtggaaatagagaacgctgctacgatcgaaggaaacaccgctgcggatgcgccggtgacggatgcggccgttgagaagaagcctgcagcgaaaggacgaaagacgaagaatgttaaggaagtgaaggagaagaagactgttgctgccgctccgaagaagagaactgtttcatctcatcctacttacgaagaggtaaaaatgagattcgatttcgtttattcgtgtttcttattaagccggattctgattttggaatttagaaattgatttttgtgttctttgttgatgcgatttcagatgattaaggatgcgattgttacgttgaaagagagaactggatctagccaatacgcgattcagaagttcatcgaggagaagcgtaaggagcttcctccaacattcagaaagctgttgcttctcaatctgaagagactcgttgcttctgggaagcttgtgaaggtcaaagcctcgtttaaactcccatcggcgtcggctaaagcatcatcccctaaggcggcagcggagaaatctgctcctgcgaagaagaaaccggcgactgtggcggttaccaaggcgaagagaaaggtcgctgcggcttccaaggctaagaaaacaatcgccgttaaacctaagactgctgctgctaagaaagtgaccgcgaaggctaaggctaagcccgttcctcgtgccactgctgctgcaactaagaggaaagctgttgatgcgaagcccaaggctaaggctagaccagccaaggcagccaaaacggccaaggttacatctccggctaagaaagctgttgctgccacgaagaaagttgctacggtggccacaaagaagaagactccggttaagaaggttgtgaagccaaagacggttaagtctccagcaaagagggcttcttctagggttaagaagtgaagttagggtttgtaggtagaagaatggttaacgatagtttagacttgtataattcaatcatctttatgcgactttgtttgcttttcttctttcagtgttcttgttattcacagttcctttggactaccccttaaatcatatatagatttcatcaatgaagcaaactcgagtttctttgcagctaatttgtgtcaggctagtgctgttcttgttgagttatagtaaacatggatgttattatacaatcaacaaacttttacaacattgaagttatacaatgaacaagtttctgttcttccttgtcaactctgcccttgttttctatgtttaccattttcagcttttggtgttgttgatgagatgttgtttagatcttttttcaactttctgaatggaatatttgagttcctgttgaatgctaaaccttgaaggtgaaagaacaatgtctttccagattgaacctgcttcacaagcagggttgggttgtctatagagctcgtaaatggagcctgcgttctcgtttaaccctcctactcgcttcaaacaatctatctcttctgtatctactagcaatgtcttttccttttctttccaacctatcagctgtttcacaatcacacttcacctattagtatttttcttatctactattatctatgcaccattgataagatgttcttgttaactcttagtgcaggagaactataactagcctaggggtgagttggggctttctaagttggtctaggatcttggcttaggcattggtcgagagtttgaaatttctcttgttcaaatcggttaagttctttgttgcattgagttaaagtataagtttatcggttccaatttcaaaactaacctttggagagccttccagcgcgttagtgcagtagagcctagcattgtcgacaaggctgcaatatgtaggaaatgcttccgcaaaccgtttgtgagatctcagctgtgatctcaccctaactgctcttctgcacatgatagctctcctacaatctcagccaaaaacactcttttaagttaataagagacaaacttgaaagatagattgaaagataagatggtgctttgtactcacctaatgcctcttataactgctaaatacgcatcgcacacaactccaactagttctattctatacggttttctcctacgaccatcctcttgaatctgttgcctttcaccaattcgttcccaatagttctctgttataactccatcatcaccaaccttgtatccagctcccattctgtaatggtgtcggtgtacgttcctcgccattgttattgtctgcaccacaaaaggtacccaagagagtgtcccatccattatcacatcccttccttcgttcagagccgtcactagcagggatgaagctgcatctgttgatgactgatgaacctatatattctcacaaaacaatttcaaacctcagaatcaacattttcatgactccgacaggttcttgaagatgtttgtggatggcttaccaattcagctgtctggatcatatcgacatggccacgagagcttaaggctctgtaaatgacatcagactctttgaaagcatcagcttcaatcaccacagagtctgctcctgcccagaatgctctgcttccatagtgaaaaccatttggttagcttctttgcttcagctacatagatatatacgtttctctatatgtatagtagaagtgataattatataatgagatctttatttactctttgagtatgtccttaagcacagtgcttttccctgcacccatcccaccacccataaggagtagcactggacttctgtctttgtgagccactggtgccataacttctgtgcactgcgagtcatccacagacgcgagccccattgctctcatttcctctaccaaagtgttgaacactcttgccacttttaagttcttagtcaccctcttaatcctcagctccctagatacatatacacacaaaatcaattattcgtcaatcactaaaacagaacacacggctttctaatacaaaataaatcctgtttctgtttcttttgttcattaccttgtagctgccatcacaatttgtttcagcttcctctttggctcagcatcagcgctcagtacctgagattcatatattatgacattgagtgctttggtctacaatgtgttgtaataagagaaaggtttttttcttttcacacaaacctgagtaatcatgagatcggcgtggctccagtgaaacgcaaagtagctgagaatgcatctttcaaactcctcaacaagcttaatgaagagggtatcagcatcaggttcttcagagaaaaagctgtaaatgtcttcttcacaacattcagatttacttatgtattctgctgctaacttgcagagatttggacactctcttctgtccttgaatcccatttgccttgctacgtttcatgtcccaaatcaggaaaaaaagcgttaacttttggtacaaggcattccttgctcatggaggataatgtgtttatatattttaccgacgtaatgagagaatctctcgagtttctcatggcctttgtgtttgtgagatgatttgagacgagggatgattttggtgtcacgtagcctccgcagacggtaatgcactgcggctgctatcattaaacctatcgatgacaccaccaatatctgagagtatgtcgaagttctgttgcctgaatattctgtcaaaaacaacaacacaatcaggaataagatttttttcttcttcttcaaaacctgtaatatcaaaaacttcacctctctgcatgacgatggattgattagtgaatggtgtgtgttttgttgaaactcagattgttcttcgtcgccattgttgttgtttcttgagtcttcattgtgttttcattttccgtaatctgtggcgaagattaaggaatctgatttctttaccaccatccaatttggtactaaaaatagctgaggagatcaattggtcttggaaaactctatttataggcagagatcccctcaaatattcaacaaaactgtatgcaaaaccaagtaccatattttaggtgtccaaaatcaagtaaaagattatcagattcgacccaatgattttttccgtagttcattgagaaattggtcaaataaaaattgatcaagttcatgtcgaaaaagatgtaaaaggaagtagcatgttacattaacctaaaaccatagtaaattataatttctaactactgagagcaaatcctctaccaaaaaatatcctaaaaacctaagaaaccccacaaatagtacaactgttacattacataggagcagtgcaagttgttctgaaatggcctgtctgattacaccgaccacaatgaaccacacgcctcacacggtcacgatcctctgcccttactctcctcttcctcggagcacctttcatagcctttggtgtttttattacattttcactgtctctctctgaatcattttctttccactgaactttgtctgatattggctctagagtttctgcataagctcttctgtaattctccactgtgaaacaactctctgtgtatctataaacgtcctcttcgcaagacaagagagctccaacggcgtggctacagggcaaaccataaacctgccaccggccgcataaacaagaacagttttcgatatttacaacgacgtttccttcacaagtcatgacttcaaattctgcttcgtttgctctgtaaactcggtgagcacgagattgttctatagcagcaagcatttgtttctcagcagaaggaacaagcacattagaccaatgcaagcttgtttcacggcgttctttcaacatatttatcaaatggcggtggatacactccatcgtttggattataggaagacctgaggtatcttcaacccaattgctgagtgactcggtaataacatttgcagttaattgtccaaaccttgttccttcaaagtatgatgatgcccatcgagcaggagacttgttttggatccataacgaagcttccggggatatctgctcgatcttgttgatttttgatttgaactcgagaactgtgagacaatgcgcagcctcccaaaaaaggtctacaagaacagagctttggaattctctttggaatcgttctgtgagataatggagacaaaagccatgaaatgctgctgggaaattagcctctaccccatcaacaacgggtctctctccgctggacaatatggtaagctttggcatattctcatctagaatcttacgaagctcggagagaaaccgatgccagttgtcatcgttctcttcgttaacaatagcgaatgccaaaggaaacacagctccatctccatcaaatccagtggcaagcagcaacgtccctggatatttgctctttaaaactgtactgtccagtgcaataagaggccggcaagcatttaaaaaaccagagattgatgcttggaaagaaataaacaagtgttgaaagcacccatcaatgggattcacatgaaccacagcgacgctcccaggattagaccttctgatttcatcacagtattggggaagaaggcgatattcttcttcaaatgacccacgaagagtagacccgcgaagagtagccattatgcgctctttccccctccatgcctgcttgtaggacagtgaaataccatgaactcgataaatctcctctagtatctcctttggtttgaaatgaggattttctttcagcttttcagcaacaacatcagcaacccactggactgaagcttgctgatgaccaagatgcgaaatcccaccacaagtatgactcccgtgaatagtccttatcgtaaaagttggagcattagaaactttggcgcagtgaatcctccatgggcaacccttgctgttgcactttgctgtgaagcgagtcttgtcagatttaatcgttcgcatctcaaaacgcagagaaattgctgcatttttaatcgctctcctacaagcataggcatcagaaaattccataccaacgaccatttcatggtcagtaccagcaccggacatggaccagcattgagagctaggagaaggcatgactgatttctgagaaaactcggtgctttgactcatctccaactctaatttatcatcggtatcttgagactctaccttatggatagcgcccgtggcttcaataacagttgatttatcaatctcagagtcatcattatcaggaaaggagcgatagccgtcattttggttaacatctaaacatgccacttctttcgtttcatcgatccctctgtggtcctggaggttgttccagagctcatttccgttacaggaggcaccaatctggaggttgatatcgagaagttgttcgaaggcggaattgtagtcatgaccaacgaagagttcatagttgtgtcgactacccgtgaaatgatcttgtctaaacgagatatcacggttctcagacttcacaagctcgtcgtttgccattctaataactaaaagaaaacactgcagaatctaatcagattattacagaagatcctgaaactcacttcttgctgtgtcctttagaaaattacctgtcaacataaaagacaatgcatggaggagacaaaacatcatcaaaaacaaacccaattcgaaaatttaccaacttgatcatgcaagcacattcagcagaatacaaacaagcccaaaatccaatgctttcgaatataagaaacagagaacagaagcaaattgcagaaaagcaaacaaacaaaaaaaaaggaggattttgaaacttacaatagaaaaatttgagaagactctaaatccattgaaactcaattaaagaaacgagtacacaattaaagggaatcgcacattgaaagagaaggaatttggcgggtaagaaattgagtgtagggtttaactgtttcaaggagaaaaatattattctcgacaaaggagaagaagaggcgacaggattgatatcatcaggttaaaaaggtaactctgtaaccttgttaggtgttttaagggtatgttggtcatttcatatccgtcgcttaagttggaaatagcatctcattggtataaacgcctaagatttatttactttattcgaggtcacattggcccaatccatgatttactaattagtgttaatgacttcaacgcgtctacgatgacgtaattttgtttttgtagattttgatacgttctacattattaatggtgattggcgaaattaacacaaaactcctcggattgaagtaaatcttcaagtttggtactaacataacttccacaaaatcccgaccaaactatcgcatcttaacgactttaacaatatgaagacaaacagaaaatagtgccaaacttctctttatgtgaactatttatcactaatgttgtaagatcaccttctctttcatatctcccaaagaaaaaaatattgccaaccaatcatttacctgaaatttcttcatccactaataagattcagctgaaacaaaaaatctccatcattacccgatgttatcttctggtttacaagtctctatgcaaatccttgagagccatcaaaggtctccaaaatcacgccgccaaagaaaatatgtgtgtcaggcgagtttgtttcaaagattcaagatgaagccttcacacaacacctcccaagttcaagttctgcaagcaattcagaaaccactttttccattgagattgtttctatcagctgcttgttgctcctcccatcctttatcatcccttttgaaagaagattgattctagatgactctgaattttcagccaggagttgttcgaaaaacttctcttcatcattgagtttcgaggccttcatcagatccaccacctgatgcctttttgcaacatttctacctgctccacagcccattgcagttgctgctccaaccgcatttgcaattgttaaggtattaacaagcggcatgttgcgtatataacctaatgcaattgcagccacaaaactgtctccacatccaacagtgtcaaccacttccacctgatgatgatcatgttttcgtaaagaaaattagatcactacacaaacttacttactctatgatacatacataataattcaattttcaacaccttgatgtgaatgccgaaaatgaacgatatttatgatctgtaagcaggtaccttaaacgcaggtgccactgagacactagatttcgtcactagaattgaaccctttgggcccatctttacaatcacccatttcgttccctttccgttcctcagaatttcttgtcctgctttaacagggttcctgatgccagttagagcctccacctaagtaattcattaaagaggtcaacaataatttgacacagtagagaaattttcagcattagcgagagatgcacaatgtacctcctcagatgttagaagaagaacatcactcattcttaagaaatgagcaagtgctctacgctcatcaggagttcccttggacagactctttccccgtggtccaggatcgaagaagatagctgtcccaactttggcagcgtaatctatcgttgacataataaaactagggctgaaatcgtcaaagtcataaccattgcagaaaagaacttttgactgtctgattgccatctttacttcatcagataggtcagtgatccaactgaaagcaggttcctccttgaaatcagctcgactaaaaaaagagaaacaactagagagaataagttcctatagtagtaaacaagaatattacccatcagaaatttgtttcacaaatagacaaaagaattaggaataaaatgggacagaaattccctgagtcaacaaattcaaagaactaggcattggaggcaagtatgacaatatcacccactagcgtttgtctcaatattagaaacaagatctctaaacaagaaagccatatctttctagacatatagcattaagaacactatcagcaagaaaaatattccgatagaaaatctggtatctacacttttttgtttccaaaaaagttctatcaaatgaaaagttatatttctaggtccccatcaccatccccatcacagttcatgaatgagatgatgcattcactatatacccttagcaaccgtttccccacataaaccagctctaccatccatagatattatcaagttttggcgtggcaaaaagcagtacctgcaaaacccatgcctctgcaaaggatccacaagaacccagcaaatgagagtttcacagaatgagctggtatctttttcatttgttcctccattcaatgcaaccgtacctattccttcctcatgaagcacatcaagaagaaactcaccatagatttcatccccaacgtgaccaatagcaacacaatgaagccccaatctagcagctgctatagccatgttacagttgccacctgcttcccagtatttctataagcaaaacagggaacatatacttcactattaacatcaacagactcaacaatactttaaccttaattcaactgaaacgagaaaacaaagattcaattttctcatcaaagagattgagctaagaagttctttcgatcaatccgaactaaggcagaacaaattatacatacaatgttcgaaattatcaccattgaaacactaagaagcagttaaagtcaagatttagaacccaaaccttatctgggggagacatagagagttcgtccatgagggccttgcgttctccacgagaaggagggggcaattcgtgaacactgagaacaatgtcgacgcagagattacccaaggttgagacatctataggtttctcgaccaccgcgacatctccagtttccccgaaagaacagatggaggaggagccatcggcgtatataaccggagatacatcagcggcgctgctccggcagcggaggcaaacagagctggtcacatgagggactctagcgaatggcgggaggagaggattagggctttgggtggagaaattggagagggataacgcatagtgaggagagaaggagtgaaattttagggaaaaagcgtggtgatacattcagaaaaacccaggaaaaaagaatgaagaagaaagagagttgattttgaatctgcttcgtcgttctctctcagcttctccttcgtcttcaatcttcttgaaatgagatactttattgttcttcaccatttgaggaagaagaaggaaggctttcttgttctgtggtaaactaaaccggagtgaaattggaatatgaaattataccgaaccgggtttgatgtggttatttaaaccgccttgaaattaaaacgggaggttttataagaaagggtttttcatctttacctgatttatacttgtcgacggcggacaccaacagctttcttcatcgtttagagctcacttcaagccatggccgccgacgaactgatgccgtctcacaggtcacacaggactcccaaatcaggtcctaccgcgaggaagaaatctgaactagataagaagaagcgtggaatctccgttgacaagcagaaaaaccttaaggtgggttactcctacttgtcctctctttgtcaatgtcttttaagagtttcttcgttgattggggtgtttataggaacaatcgattttacttggttctgcaggcgtttggtgttaaatcggttgttcatgcgaagaaagcaaaacatcacgctgcggagaaggagcaaaagcggcttcatcttccgaaaattgatcgtaattatggcgaagctcctcctttcgtcgtcgtggttcaaggccccccaggagtactgtctttgagactgatgttgcattttcactctcggttttgcttattttctcacaagttttgattgttcctgtaggttggaaagtctctcgtgattaaatctcttgtgaaggaatttacaaaacagaatgtacccgaggttcgaggacctattaccattgtacaaggttctgtgctgtatttgtttattgcttcttcttcttttctttttttgtgtatgcatctatcttgaattttagggttggaatttactcctgctctcattttggctggtcatggatttaggtaaacagagaaggtttcaatttgtggagtgcccgaatgatatcaatgcgatggtggattgtgcaaaggttgctgatctagccctacttgttgtagacgggagttatggttttgagatggtgagacactatttttctgtagctttctcctctttccataatgatctagattgtgaagaatctttcttatgtatacaagatcactgttgggccttttttgtgatctacatttttttgtgataaacaactaaggtttgacacgtgaactgtcttttaatctgtttcaccctcaggaaacctttgaattcctcaatattatgcaagtgcatggatttcctagagttatgggtgttctcactcaccttgataagttcaatgatgttaagaagctgagaaaaacaaaacatcatctcaagcatcggttttggactgaaatatatcatggagctaaattgttttatttatctggtctcattcatgggaagtaagtatctgaattggtgcatcaccaacatgaatgacatgtgcaaggagcgtctgaggttcacctttgatttatcttttattggtaggtatacgccgcgtgaagttcacaacctcgcccgctttgtaattgttatcaagcctcagccattgacatggcgaacagcacatccttatgtgttggttgatcgccttgaagatgttacccctccggagaaagttcagatggataagaaatgcgatagaaatatcactgtgtttggttacctacgtggttgtaacttcaaaaaaaggatgaaggtatgtcatcactgtccttagatactggttcatctgaacctttttctatggcctgaatccttgacctatttgtttgcttagcagtatgtggcgcaaaatactcaatattttgggaaaagaccaatttttaaattgagcctagagatgagctgatttgatgtcaatttgatgttatatatacagtcttccttactggtattcgtccatagattcttagcgattcacatatatttagaactacctaaagtaacccgttgtattcacacgtagctagttctctcatcgagtgaagttctaagaggaggagcataaatttgtataccttctgtgttgtactgaaattttctttgcttctttatctagtggaatgattttgtgtgcaaaatggtatcttagctgttacagtttacctttctttcatgtttaacatgcatctgatataatttcttatgattatcaggttcatattgctggagttggtgacttcattgtagctggggtgactgctttaactgatccttgtcctttaccttcagctggcaagaaaaaagggctgagggacagggataagcttttctatgctcctatgtccgggattggagatcttgtgtatgacaaagatgctgtttacatcaacataaatagtcaccaagttcagtactctaaaactgacgatggaaagggagaacctactaataaaggttattttatggtgttttattgtcatatgcttggtctgtatgtaactgcagtagtctcttttgagtgtcttgctcatttcatgtttaaagaatttctacatattttaaatttggtttacatcattctccatgatagaaacccaatagtttttgctcactttttgtagctctagcatctctgattgtgacattttttcttttggcaggaaagggcagagatgttggtgaagatttggtaaagtcgttgcagaacacaaagtattctgttgatgagaaactagataagacattcattaacttttttggcaaaaagactagtgccagttcagaaacaaaacttaaggctgaagatgcgtatcactctttgccggaaggttctgacagtgagtctcaatctggcgatgatgaggaggatatagtaggtaatgaaagtgaaatgaagcaggaaactgagattcatggtggaaggttgaggaggaaagctatcttcaagacggtaatttccttactggatctgattttcgtaatgcttctatttacattgcagtcacatcttcagttggataatactagtgactatgagttcaagagttagtgaatgtgaatttgcttttttcattagtttgtattcttcttgcttcattcaccactactgtcagacaatttggaaagaaatggtgaaacagtgtaagtaggttaagtcattgcagttctagtgggatcactgtagtgaaaaagtttttgggaagcggtatgagtatctatctgatgttgactcattatatcttcacaattaagtggttgtctccatgtctggctgtgtgtgacgcaggacttgaatgaagatgattttgaggaagcagacgatcttgaattggattcatatgacccagatacatatgattttgaggaagcagacgatgctgaatcagacgataatgaagttgaagatggtggagatgactctgcttccgattcagccgatggtgaaccaggggattatcagatagatggtatgttaattctatgtttgaatatactttcgtatagataatgtcatgagagttatgggacatctatgagaaggttttagattttcttctgaaagtacattgtcctttctcttttgagtttttgttcatttctaaacatttttaccttacatggtctgtcagataaggactctggtaacatatcacaatggaaagcacccttgaaggagatagccagaaagaagaaccccaacttgatgcaaattgtgtatggagcatcatcattagctactcccttgataaatgagaaccatgacattagtgatgatgacgaaagtgatgatgaagacttctttaagccaaaaggagaacaacacaaggtttgtcatctcaagtttgacaaaatttggtgatccttttaatctcttggaaaggaatattaatcgttattttttcatataaatccagaatttaggtggtggattggatgtgggatatgtcaactcagaggattgttctaaatttgtgaattatggatacctaaagaattggaaagagaaagaagtatgtgagagcattcgtgatcgatttaccactggtgattggtcaaaagctgctctgagagacaaaaatttaggtactggcggtgagggagaagatgatgaactttatggtgattttgaggatctagagacgggagagaagcacaaaagccatgagaacttggaatcgggtgcaaatgaaaatgaagatgaagatgcagaagtcgttgagcgtaggctaaagaagctggctcgtcgagcaaaatttgatgcagaatatcctttttacccgaatgacatcatttaacttatgtaagcacgatatcatttgctgtctttcatctctgttttaggccaataagttgtctactgctgtctgtgtttattattctctgaaacaactcacatgaaaatatggtgaagaattatagcttagtttatcaaatgggtcactaagttctaagttcatatatcgctctggtcagggagtttcatggttgctgtttagatattcttcctctgcatgtcttatatgtcatatttggttttatgctactgtttctatttgcattttggcaagttgcttttggttcaaccttaacttcgttgtacaagcaataaatccgagttagaggaggatgacaatgataaaggtgatgggaacaatcctcgtagtcaagccgatgaaccaggatacgctgataaagtaagtagattttgctgcaggcagctaagatatatatccactgttttaaaactcactatttgctttgcagttgaaggaagcgcaggaaattacaaaacagaggaatgagttagaatacaatgatcttgacgaggaaactcgaattgagttagcaggattccggactggaacatacttgaggctggagattcacaatgttccttatgagatggttgaattctttgatccttgtcatccaattctagttggaggtattggtttcggcgaggacaatgttggatatatgcaggtaagctacaaagattttcttttcttctggcccaaatgaaggtccaaatgggcacactgctcttttcagcttaaaagatgaaatatctttttcttatttaaaggtaattgaaagatgatattttatttgatagatgcttatcgtatcacatgtatctttctacttaggcccggttgaagaaacataggtggcataagaaagtactaaagacaagagatcctattattgtgtctattggatggagacgctatcagactattcctgtatttgccattgaagatcgcaatggcaggcatcgaatgctcaagtatactccagaacacatgcactgccttgcttcgttctggggtcctcttgtcccacccaacactggctttgtcgctttccagaacctgtcaaacaatcaggtatgtgattgcactctgctgcttgagattttgatcccaagttaacttagagtgtgtttgaacttttgtgatagagaatcgatcttctgtaattcaaagtgcgtcttcatgaaatgggtagtgtgagtttaggactgaccaatagtgaataatcgtatggatacttagtgtgttttgaatgagaaaagttagaagatgctttcttcctcctggtcgatgtcacaacctaaaatgacagttatgctttcatattgttggagcaggatttagcacacatatcagtccattctgctccattgtttattgttacgatatcatgcaactgttcaaaattatttctattagttagaaacccctaatggcccacatatcatgtaaggggaatttatcggttgtttctttggttagatctgttgtattcttctcctgctgagtagctctcctggtgtgtgcttcaggcaggatttaggataacagcgacttctgtagttctggagtttaatcaccaggcccgtattgtaaagaaaatcaagctggttgggactccgtgcaagatcaagaaaaagactgcatttatcaaagacatgttcacttctgaccttgaaatagctcgatttgaaggttcatctgttcggacagttagtggcattagaggacaagtaaaaaaggtatgcttttgatctccttgtaattcctagtttttccacatatgttggctctccggttttctaataagctttcttttgtcaggctggaaaaaacatgcttgataacaaggctgaagaagggattgcgaggtgtacctttgaagatcaaatccatatgagcgacatggtattcttaagggcttggactacagtggaagttccacaattttacaatcctctaacgacagccttgcaaccccgcgataagacctggaatgggatgaaaacttttggcgaactccgtagagagctgaatattcctattccagtgaataaggattcactctacaaggtaaagccataaagtgcggtagcattgatatatatttgaaggtctaaacctaaagtttatgtggggaactaaagagcggctatctcgctaatttctctctcttttgtgtaacattttgcaggcaatcgaaagaaagcaaaagaagttcaatccactacagattccaaagcgtctagaaaaagatttaccgtttatgtcgaaacccaaaaatataccaaagcggaaaagaccatcactagaggataaaagagcagttataatggaaccgaaagaaagaaaagagcatactatcatccagcaattccagctgcttcaacatcacacggtaatgttttaaaaactaatagcatcatactatcatgcagcataaaatctttgattgatcttgtctgacttgatggagaaaaaaaaaacatgcagatgaagaagaaaaaggcaacggatcagaagaagaggaaagagtatgaagcagagaaagctaagaatgaggaaataaataagaaacgtaggagagaagagagacgggacagatatcgtgaggaagataaacagaaaaagaagacgagaagaagccttgattaatttattaataccttatttcaacttttgtgtaatactaaaagttttgttttcatctctattgttcttctgtgttgttttttactttaaaacttatgaatttatcaatcacatttctcgaatgatcactttctaccccaaatttttgaaatactgaaacttttcttggttactctaattatcactacgttttgtgcaagcaaaaatagattagactgttaggatttgctaatgaatgttaaaagaaaaatgtaattaaacagaactggtaaaaagctgacaaaactggaatgggtgggaacatcgtcttccccttccccttcttcatcttcttcgtataaaccctaaaccctaaattctctctatcggttagctatgaacaaaacagtcgtaagatgtcttctttctcgttcacaccatcctttgattcatttctccaccaatttatctcttctccacagagttttcacctgtagccgttatctcactgctaggtttatgtccactcctccgccggatgacatgtttggtttcgacgatcctttctcccccagtgattcgcgggaagttgtggatttgactaaagagtactcctttttgcacgattctcttgtcgactatggaaacgttaatgttcatcaagtagtgcccattattactcaatcctctattgacgctagagccattgcagatgctgtttctggtgtcgacgatgtgtttgggagaaaatcccagaagtttcttaggcaatttagagagaaattgagtgagagtttagtgattgaggtgttgcgtttgatagcaagaccatctgctgttatcagtttcttcgtttgggcaggtaggcagataggttataagcatactgcacctgtgtataacgctttggttgaccttattgtacgtgatgatgacgaaaaagttcccgaagaattcctgcaacagattagagatgatgataaggaagtgtttggcgaatttctcaatgtgttggtaaggaaacattgcaggaatgggtcatttagtattgcgcttgaggagttagggaggctcaaggattttaggttcagaccttcaagatcgacttataattgtttgattcaggcatttctcaaggctgatcgtttggactctgcatctttgattcatagggaaatgtctcttgcaaatcttaggatggatgggtttacacttagatgctttgcttattctctctgtaaagtaggaaagtggagggaagctctaacattggtggaaacggaaaattttgtgcctgatactgtgttttatacaaaattgatatctggtctgtgtgaagcttcgctttttgaagaggctatggatttcctgaataggatgcgtgctacttcctgccttccaaatgttgttacatattcaactttgctgtgtggatgcttgaacaaaaaacagctgggtaggtgtaaaagggtgcttaatatgatgatgatggaaggttgttatccaagtcccaagatttttaattctcttgtacatgcttattgcacatcaggagatcattcatatgcatacaaattgctcaaaaaaatggttaaatgtggtcacatgccaggatatgttgtctacaatatattgattgggagtatttgtggtgacaaagactcccttaattgtgatctgttggacttagctgagaaagcttatagtgaaatgcttgctgcaggggttgttctgaataagattaatgtcagtagcttcacacggtgtctttgtagtgctgggaaatatgaaaaggcatttagtgttatccgtgaaatgattggtcagggatttataccagataccagtacctattccaaagtccttaattatttatgtaatgcttcgaaaatggagttggcgtttttgttatttgaagaaatgaaaaggggtggccttgttgctgatgtctacacgtatactattatggtagatagtttctgtaaagctggtctaattgaacaggctcgtaagtggttcaatgaaatgagagaggttggttgtacacctaacgtcgtcacatataccgctcttatccatgcttatcttaaggctaagaaggtcagctatgcaaatgagctgtttgaaacgatgctgtctgaagggtgtctccctaatattgttacatattctgccttaattgatggccattgcaaagctggacaagtggagaaagcctgccagatttttgaaagaatgtgtggcagcaaagatgtcccagatgtagatatgtacttcaagcagtatgatgacaacagcgagaggccaaatgtagttacatatggagctttactggatggtttctgcaagtcgcatagggtcgaagaagctcgtaagttgttggatgctatgtctatggaaggttgtgaaccgaatcagattgtatatgatgctctcattgatggactctgtaaggttgggaaactagacgaagcacaagaagtgaaaactgagatgtctgagcatggattccctgcaaccttatatacttacagttctctaattgatcgttatttcaaagtcaagcgccaagatttagcctcaaaagtattatctaagatgcttgagaattcctgtgcgcccaatgtggttatatacactgagatgattgatggcttatgcaaggttggtaaaactgatgaagcttataagcttatgcaaatgatggaagaaaaagggtgtcagcccaatgttgtgacatatacagcgatgattgatggatttggaatgattggtaaaatagaaacatgccttgagctcttagagcgaatgggttccaaaggtgttgctccaaattatgttacttacagggtcttgatagatcattgttgcaaaaatggtgcactggatgtggcccacaatcttctcgaagaaatgaaacagacacattggcctacacacacagcaggatatcgcaaagtcattgaaggattcaataaagagttcatagagtctctagggcttctcgatgagataggccaggatgatactgccccttttctttctgtatataggcttttgattgataatcttattaaagctcaaagactggaaatggctcttaggcttcttgaagaggttgcaacattttcagccacgttggttgactacagtagcacatataactcactgattgaaagcctttgccttgctaataaagtggaaacggcttttcagttgttctcagaaatgacaaagaaaggtgtcattccggagatgcaatcattctgtagtctaatcaagggacttttccgaaacagcaagatcagtgaagcactattgcttctagatttcatatctcatatggtttgtcctctctgactctttcttctaagtttgaaaactgcagtagtcgttaatgtttgtcaagctttatttctttgcgatgttcacttgaatctatgtgtatttgactagagctaattatctagtggaagaaaaggcatttaccgtggtagtttatattgtttatttgttctctccatcattgtgagcagtgataatgcagattggtactttttctggtattcattacttaattgtctcaattttgcaggaaattcagtggatagaagaaaagaaaacatctgatgggacttaggcatattggtttatgatgtctagttctgatattctcagctagttggtgcatctgatgttgagcatcctagtcaaaccctgaaaattcgaaggttggaagctatggataatgcatgaggcgtgtaggatttttggaccaaaattctttgtaagttcattggttttagatcacatagtcagaaatcatgtcttttccggtttttcctatgaatatgtttatgtatgcaggttctgctttgtacatgatgtgtaaaattatacttgtaatgactctcaacagagttgaatggcctcctttgaacactctatgcatcagatatgcagagctcagcttctctctcagatcacagtatttgcttcttctggggtgactcagtttcccactctttagtagtccaaacatttttagatgctgtgttttctgtttataagaacctttacttcactggtcaattctcagctaatatatgaaagataaattcatacttgttttgtaatcgatacatgatatgttctttactgttgcacctttccatattgatgatgatgttagggatgttgtatcttgaaaagaacgtatttgcatagatgagtagtagtatgcaatcgatgcatgctttgtattgaaagtttattactctattgctaatccaggcataacgtgttacagttcctgcggaatgagcttggatgaacacggacacacaggaaagggaccaagaagatctcaaagcattaacaaagtatctctgtccgaagacacaaggtaaggcactcatcgttttgttggatttttacctgagaagacagtccatatagatgtaggtggttacatgacgatagttttgcaggtaaaatatggtgacaaaaccatcaaaagcttttgtgcaaaatggaggagttctatctaacgagaagctgggtttttttgcttgaaacagtttggttgctatcacacttagcatgacaacacactacctaatgaatgaagaaggcccaataaggtgtcagatatgatatgcataaagccagtgtttgtgtataatacgggacaagggccctttactgtacatatagatttattttggtttaccctacagtctacagatcaatgtatactttattttgttttttgttattaaataagatgagagatcttcccctctgttaatatgtctggcttgttttggattttatgttaatgcactggctttctgacattggaacactatctattatggaagatcattttaaaggattaatctatctatgtatgtatgtcgtttagggaaagaaaacaatattggcaaaagataacaatagctaaaacttgtaaagaaattcaacttttccttttttcattagtatagaaaatcccaacttggtaaaagaagacttgagagtcaaatgttgttactataaaacaaatatgaccaccattgaccggaaaacgagaagattttttcgtaattcgttgttaatttctctcgttggtcagatatatttcgatatctattattattatttggtgattttttcatatctggaaattgagtaagtaacgatttttttttttaaaaaaaaaaaagagagagagagagattaggaaacaagaaagcagaaagagaggagtgattagagagagagagagatcaataaccaaagaagaaggaagggaaggggattagattacaaaaacgccaaagctgttaactttttctttttggccatttcctatttctcccttcgtctccatcaagcaaccctctctctctctctctctctatcatcatcatcgcagaggagacgatcttacgcgacgaatccttgagagattgagagggacaagcgagttttgtaggtgtctcagaccggttatttttttctcttttcttctttttatctgaatgattttggatttcgtagacaattgtgtggtctctagattcatgctttctaggcctttttaacgcatctttgctttcttagatttcatgttttacaattcgatttcgtttctgattttcttttcatctgggttttgtttctgatttgtaaatgttcattcttttgtggattgtgatccttgagtattgttttcttctggccgtatttgctggcaatatgaggttgtcgtccctttttttgctttttggtttttgcatgtattattatccatcttctgcccataaacaatgttctcaatgtagttggatcttgtctttttttttctgagaattttgattttatgaaacttgtaattacattttttttttctcaatcaaattgctatcaagatgtaattacattgctgaaactgtgtatagtagaagacatgtttttggctcacacatctggtcctatctcttttgcatctaaattcagttacttgatctatttcaggacccaaggattgttgacggtctatattgaacaagtaaacaaggatgcgtaagtggatctgttgtacatgtcaaatagaagactcaaatgaagagcaacaactgaaaagttcacagcagcaatctgatggtatgttatctagatgccacctagtacctcaagtattctaattatcacgtactagtggcttccattcccacctttgtataacagttatcatatcttccaactgaggggaataagttgacatgttgtcaacactttaaacatcttatgacttcgtgttgtgtatcttaaaaattgattttctgtttatgcttattgtgttgagtatcttcatgtatgcagcaaatcataagaattcaaaaccagcacctgttgcaaaacatgaggttaagaaggaagctcttcccatcgaggtccctcccttgtctttggatgaggtcaaggaaaagactgaaaattttggatccaaggcactgattggtgaagggtcttatgggagagtatattatgcgacactaaatgatggtgtggcggttgctctgaagaaacttgatgtcgcacctgaagctgaaacagataccgaattcttgagtcaggttattaatctgcattttgttaatgaagcttgtgtttacatcctctttcaacttgtcatattagttgtttgctcttccttaggtttccatggtctctaggctgaagcatgaaaatctcattcaattgcttggtttttgtgttgatggaaacctccgcgtccttgcatatgagtttgcaacaatgggatcgttgcatgacatcttgcatggtaagattagctttttggactttaagattctctgctcagttctctaactaaataaaccccctgttttgaccaaaagtattcatatcggtgccaaacgataatttgtccatgtaaggttgcttattgtgcgattacaggtaggaagggagttcaaggagcacaaccaggtccaacacttgactggataacgagggtgaagatagcggttgaggcagctaggggtttggaatacctccatgagaagtctcagcctcctgtaatccatagggacataagatctagcaatgtccttctttttgaagactacaaagcaaagattgctgatttcaatctctcgaatcaagcacctgataatgctgcccgtcttcactcaacaagagtcttggggacttttggttatcatgctccagagtaatgtttcttaagcccttattgttttgtattcgtatgaatgtctaataaacagctaaaatgttaatccgccctcttatgcagatatgcaatgactggacagttaactcagaagagtgatgtgtacagctttggtgttgtacttctagaacttcttacagggaggaaaccggtggatcataccatgcctagaggtcaacagagtcttgtaacctgggtgagtatacatacgttctggactataatgtttcgtattaattgtccccttggctttacaatttcattttattggctgtgctcaggcgacaccaagactcagtgaagataaagtgaaacagtgcattgatccaaagctaaaagccgattaccctccaaaagcagttgctaaggtacctcgatacatacaatagtttcatattcatgtctattaaatttccgttttcgatatattcgtgtctttgagattgacagaatatgttatgttggcaatataatgcagctagcagcggtggcagcattgtgtgtgcaatacgaagctgagtttagaccaaatatgagcatagttgtgaaggcgctacagcctctcctgaaacctccagcagcagctccagcaccagaatcttgaaacttttaaatcttttttttcgctgcaactatcagaaagaatctacatagtaccatttgccataaatgataatgaggttggttttgagagtgataactactattattaaaagatgatattatgtttcatttgatgatttgattttgcacacaaattttgttgtgctagaaagagagagagataattgatggcaaagtttattgttgccgtttgctgagtgggagagaaaggaactctgattttatttatatgatccctcttattcctatttctctctaccttttggattaataacctcaaaaattcgaataacttgatcattgaatatttgcgttagtcgtgtatgataattacaatgttttagttttcttaacaaatcgttaccttcttcttctttttgtcgtctcttatttatgaaaaaaaaaagaaaaaaaaaaaaagaaacaagcagcataacaaagctaaaaggcccaactgaaaagtaaataaacaaaggcccaaccaagcccaagagaaaaccaatgtcagttttgtttttttgtttttgaatttcaccggttattcattaaagaattgtttggcccgccaacaacatacgtgtccatatccacagtctctcacctctcgcagtgcccaaacctcttcttcttcttcttcttcttggcttctcctcctccttttccaatttcaaaacccaaaaacaaatcagcatggccatggctacgcacttcaccttccctttcaactatgttgtttccgaaggctctcatggaagaagaagtttcgtccggaagctggtcagagctgtagcctccggagattccgtggctccggcgatttcggaagaatcaaaagtgaaattgggtggctcggatttgaaagtgacgaagcttgggatcggagtctggtcttggggtgataacagctattggaatgatttccagtgggatggtcagttctcttaatttcgattctcaattttatacattttgtgaaacaaaagtattgaaatgaattaaaagttaatctctttttttttattctctgtgatggtagacagaaaattgaaggcggctaaaggtgctttcgacaccagtcttgacaacggtattgattttttcgataccgcagaagtttatggttccaaggtaagcgtaagaaagctgatcactcttcgtaaattggtttattctgaaactaatttggaaatctccatcttttatagttttccctgggtgctataagctctgaaactcttttaggaaggtaatttgtttcgaaaacatataacagtgtgccacactatcctcatatatattcctcttgactgtcattggtgagattttcgaacagattcatccgtgagagaaaagagagatatcccggagctgaggtttcggttgcaaccaagtttgctgcattgccatggcgatttggtcgtgaaagtgttgtcactgcccttaaagattccctttcccggcttgagctttcatctgttgatctctatcaactgcattggtatgcacccctaattcatgttggagtaaacaacactagaaggtaacaatctcactattttcttaattatatttcaggccaggactatggggaaatgaaggttttttcttaagagcagcactgaatctacttttaactagagttcgtgtcacttttggtgattatatcaaatccattgacattgtgagttatgtttttaggatatcttgatggtcttggggatgctgttgaacaaggacttgtcaaggctgttggtgtttctaactacagcggtatcatttcaatctgttttttccttctcacggatgcatatcttatgaactttcaaattcattatacttactgttgtgcgatttttcagagaagcggcttcgtgatgcttatgaaagactcaaaaagagaggaattcctctggcctcaaatcaagtcaactacagtctgatatacagagctccagagcagactggtgttaaggctgcttgtgatgagcttggagttactttaattgcatattcccccatcgctcaaggtaatctgcctttatctcctttgcgttgtgttcatgtctatcgttctctagaaaagttgcatcaggctcttatagtattgacgcaaaacaacctggctaatgaacacatacattttccgttatttgctcttttgtcttttttccaggtgctctaactggtaaatacaccccagagaacccaccctcaggtcctcggggtcgcatttatacgcgtgagttcttaacaaaggtaatagctttcgctgcatatatctaagttatttaacacaggcgtgtatgtctgcaaaaccatagttacatctgaataaccctcgagtcttctaagaaacagatttttggattttgataacttgtatatctcactgttataaacctgtcgattttctcctctaatcctgcttttttcttagtaagttctgttttgagacaaagtatacacttttatacaaaacacaacaccaatcaatgtacctatacagaaattatcaaaaggtagcataatagtgccaggtttcttcactaactttgacttgtgacgttacagcttcaacccctgttgaatagaatcaaacaaattggggaaaactacagcaaaactcccacacaggttctttcccttttccttatgcactctgtttttctttttccctgtccaaaaacgttaagaaaaatacaaaagagtaattgtgtgatccatattaagctatccaagtgtacaccctcgattttgcatcaaatcatatgccttcttgttttgttgtgttgtgctgtgcagatagcattgaactggttggtggcacaagggaacgtgatacccatcccaggagccaagaacgcagaacaagccaaagagtttgctggagcgataggttggagtctgacagacaatgaagtgagtgagctacggtccctggcctctgagataaaacccgtcgttggtttccctgttgagtatctctgaactcttaacactctgtttgtagcatttgtaaagctcaaaagttacaatatgaactaaaagtagtatatagatgatggtaaagtgttgtaatacatatgcaaataaaatctttttgcctctttgagaactttacagtagccaaaaagaaagaggagggcctttggcatatgcttcaatagatctagttagagtcagacgtcaaaattcatctgcgttttaacctttctgttgactagaagaaccatttgatgcttacctcttttcatatcagcttcaagcaggctttcttctcactatctttgtgctttcaaggatttagatttagtctatagcttctttctgagtcaatatcttattaatttgagaaaatatctattgaatatagttgttttttaagaaaacttggaaaacgagtctgttatcataggtacacaagtgatgccgaaaaggtaacatcatcttgatagttctcaaaagctcagtttcattttccattttttacaagagacaaatgaagaaagcaagcagagcattgttacgttgtgttgctttcactcaagcaacactgaaagaagtggctgcgctctcgacaaatttcctggctcccttgaaccacctcttgtctccagcttgtgctttgcagatgtaaagcttccctccattcacggttgctgtgatcagctgatgcttcccaccttcgtctccatcagccgttcttgtcaacacagacaagtaatagtagggtttcccaccaacttcctgagatgatgactccagaatgtttgctgttgccactgcattgttgtcaaagcctccctgtaagaacatcagattcacacacacacagagttagaacttgataactcactccacctaaggtaagaatctttcttgtaataatcatcgactactagagaggtctttgaaggttatcagttaaaaaagcttttgagttttgttttatcagttaacaacgagggagacaatgtgttctctctttagtgtttgattctcttattctattctatcagcttctggtttacttataacaaataacaatgggaagtttacctcagaggcagtctcaccgaagtaagcttgtttccctaggaggtaattaacctgcataaagcccaaatcaccactcagtctcacacttcattagattttgtaacatcaatacctttacagatgatggagtaaagcaaagacctgagagaggaactcttcgggagaaccgtaatcagtgatggacttcttgtcggtaggagtgaccatgacattgagattgctagtagcatcgaagttgtcttcgaacctaaggacttgtcctggatactcaatctctttgcttgggttccattttgctggaacctgcactttgaacccatctccattgtatggcaagaagtctgtgttcgtctttggcttcccaaacacgtttgctgcaacaaaccaaaaaataaaaataggatgaaaaaaacacaatatgttcttcaagattcttcttcaagaaaatgaaatgatccaaggttattttattttataaacgcaccagcttcaccgtaggcggcatcagcaggagatactttggaaccaacagcagcggcgccgacgaggagagtgagagcaagacggcgggagacggcggagttatcgtcttcatgagactgttgagctttacagatgatctgaacaggtttggagagcgacacgtgacgctgggatgaggaggaagatgatgatcgtgcggctgatgaagccaatgcgctctggtgtaggaaacacgcactgtacgccattatctctgtctctctttctctctttctctcttctctccttgtgtgtgattaagttatgaaagtgttttttgaggattgttggtgtgttaatgtatggaggtgaaaccaagagtgaagctgattggttggtttcggatattggaggatctctctgttgatgtccagataaggctgttgaggtgttgtattgtgccacgtggcagatacgctttcttcttgttccacgtcagattcataagccttcgattccatctattttagtattttactctaaagccattccatgatcaaatcttttttctgctcacactaagacactacagtactggatcaataatggcttttcttctttcagttacatgcgtatatcttaaaacgtttataacgatagaacaacggaagcactaagtgagaatgaatacaaattatctaacaaaattgttgaaattaagaatctgttacacttatgtagacaagtgccatttcaaaacttggtttcatgacaaacaccgaaatgctcattaaaacaccaaagtggaatgggcatcatacttcccttcttctttcggttttgcttaggttggtgcagtatttgagccacactgctcattgatcggaaggtatgctcggtttttcctcaatcaaacctgacttgtttccctggctcaatacattagtggaaggcattttctcttccatattgttaaccgaatcaaagttgttagctaatttgcgctttttcttttttggtttcttagctggagcagtgtttgagtcaggttgctcattcccactgacaagattcgaaggaatgctctctttctccttacccggatccaacctgttgactgactcaggatctttttgctcatttccgtttgccagatcagaaggcttgttttcttccatgttccccaagtccaggttgttgactgatttgcactttttggaccttgatcgcttcttttttggttgcttagcagcttccattggagcagtggttgggtcaggttgttcatccccattagcaagattcaaaggcaagctctcttccatgttaccataatccgagttgttaggtagcctaagctctgtttgctcattctcattagccagatccgaaggcgtgttctctttcatattccctaaatccaagttgttggctgacttgggctctgtttgctcattctcatttgccagatcggaaggcttgctcttttccatcttccccaaatccaggttgttgcctgatttgcgctttttagacctggatcgcttcttttctggttgcttagccgctgaagcatcttcacctggagcaatgtttaggtcatgttgctcatttccattgacgagattagaaggaatgctcactttctcgctacccagatccaacttgctgactgacttaggctcttttttcttattcccatttgccaaatcagaaggcgtgttctcttccatgttccctaaatccatgctgttggctgagttaggctctgttttctcattcccattggccagatcagaaggcgtgttctcttccatgttccccatgtccaagttgctagctaacttcggcaatgtttgctcattcccaatagccagatcagaaggtgggttctcttccatgttcccgaaattctcttcaatgttccccaaatccacgttgttgagcgacttgcaccttttggactctgactgcatcttcttttgttttgtagccgccgaaacatcttccactggagaagtgtttgggtcaggttgctcattcccatcagcgaaatttgaaggcaagctctcttccatattacccaaacccaacttgttggctaattcgagctttgaccccattggagtagtgttcgggtcatgctcccgaagctcattggggttagtggaaggtatgtcatctctgggactcgttgcatctaccactgaagtaataggttgcacagctacagtttgaactgaagattgtgcgaggctatcatatgagagaacagaagctattgcatacatagaagcaccgagatgaggcggcaaattttcctttggatgtttaacctacaaatgcatatggaaggttgaaaaggatgacattgccaaaataaaataatgaaaatcttaaatcgtagtgataactcatgtaccttgaacaaaatggacgccattagtctctctctcaagatgtacatttgagcaatttcaaaagctgtaaccttgaaaggcagccaccgatccactactacttttactgaattttccggcccaagcattattttctctccaggcctaccaacttctttatctatgtccatcgcgtctcctacatttcctactttgtggtcttcctcgtcatcactcttaacagcatcatagctctctgtagtagaaacggctatctctcttgaaaacagtaacacaggtatagtgggaagaacagtgcagctccttatcaccacaccccaatctcctcgagtgatctcatcaaaaacaattaaagcttcgtcaaacttggtagaggacatgtcaacattgtttgatagcgagggcacacggactttggctccagcaatagtctctattacagaccttgtccgattcttggaaagtgggcacatccttcccaacatagggtacaatccaactgctataacagcacggagaatacccggatcatgagcattcaggctacagtttgagctgctgctaggtatgaccccatgtctattaagttctccctgaagcttgcgacacagatcatccagcctcttcatgacaacttgggagatgaagtacttagaacaaaactccttagcttgaccacttgcttttgcatttttccagcactgaaaagccgcgacagtggcaagatgatcactgtggtctccataaagcgatgcaagttcatgttttgcggcagcagccttttttctatcacctggcgacaagggcatggtaaacggatccttctcgtcagctgcacatgccagaataagtgctggatccaaacagttcactaatatggcgaaataaatcatcttgctaatccgaggatgaactggaagctgaccaaatttctgtccaagttcagtcagctcttcttcaggagtcaaagctccaatatctttaaggatgattaatgcattttcaatactttgagcaacaggcgggtccatcaatttctgaaggaaatcatttacgttgcaattcggatccagcatcttaacctgaacatattgatattatagaaggacatcagtaagaaatcaagaacgcaagaagtctcaaacaggacgctaccatgcatgaatgacaattaaacatcatagaaaaaatctacgttagaaagaacagcacctgcaagcagagttcatccactggcattctcataacttcaggaactctatattcgggcaaagacgctgcccggagtttagaatatagatgataacagatgccagcttggcaacggcctgctcggcctgccctctgttttgcatttgcttttgatacccaagaagattggagcgttgacacgtcattatagggatcataactcttttccttcatccgaccactatctatcacataaaccacatcatcgatggtaactgctgattcagcaatatttgttgcaagtacaattttgcgacaaccacgaggggggcgattaaaaactttcttctgttcctcagctggaacccttgagtgaagacatagaatgatgaatttagcactatgtgcaaaaaaccgatcatcaagcaatttctcttttgttttgcttatttcctcccacccaggtagaaaaacaagaatggcaccatcttttgaatcgctgcatattttcttcattaacttcactataagacccacatctacttcttctggtttgattgtcgccatatacttatcaagtaagtcttgtgcttgctgagaattggactggatgttacccgcatgttccctgattatttgagcagtttcaaattgattctccttttcagctaattctaatgctgttataccttctttggacttcagtgtacagtcagcgccgactgaaaggagcttacagacatcgctaaccctgccctttccagcaaagaccataagtggtgttaggcctgtcgttgagttctggtaattgtaagcctcatgacttccttcagatgaaactagatctacgagacaatcaaactcatcatttgtccatgccaagtcgattgcttcatccaaagaaactttatcttcatctttaaaatcacgtttgacagcagaaagtagatgactattcttgtctgaattcaggacagagagcgcatcatccagaaaaaaggttctcacctgtagatacggacgcaaaaatattgttttccaatacaatgagactaagaagcaaattctataaagagacggatagagagagacataaagagagaggaattc

SEQ ID NO: 23 - protein, 587 aa, Arabidopsis thaliana
MDLCFQNPVKCGDRLFSALNTSTYYKLGTSNLGFNGPVLENRKKKKKLPRMVTVKSVSSSVVASTVQGTKRDGGESLYDAIVIGSGIGGLVAATQLAVKEARVLVLEKYLIPGGSSGFYERDGYTFDVGSSVMFGFSDKALKAVGRKMEVIPDPTTVHFHLPNNLSVRIHREYDDFIAELTSKFPHEKEGILGFYGDCWKIFNSLNSLELKSLEEPIYLFGQFFQKPLECLTLAYYLPQNAGAIARKYIKDPQLLSFIDAECFIVSTVNALQTPMINASMVLCDRHYGGINYPVGGVGGIAKSLAEGLVDQGSEIQYKANVKSIILDHGKAVGVRLADGREFFAKTIISNATRWDTFGKLLKGEKLPKEEENFQKVYVKAPSFLSIHMGVKAEVLPPDTDCHHFVLEDDWKNLEEPYGSIFLSIPTILDSSLAPDGRHILHIFTTSSIEDWEGLPPKEYEAKKEDVAARIIQRLEKKLFPGLSSSITFKEVGTPRTHRRFLARDKGTYGPMPRGTPKGLLGMPFNTTAIDGLYCVGDSCFPGQGVIAVAFSGVMCAHRVAADIGLEKKSRVLDVGLLGLLGWLRTLA

SEQ ID NO: 24 - DNA, 20 bp, Artificial sequence
  single strand DNA oligonucleotide
gccctgggaagagtgttttt

SEQ ID NO: 25 - DNA, 24 bp, Artificial sequence
  single strand DNA oligonucleotide
ttgctccgtgtccgttgttaactt

SEQ ID NO: 26 - DNA, 24 bp, Artificial sequence
  Single strand DNA oligonucleotide
ggcgatcgtgtgagctcattgctt

SEQ ID NO: 27 - DNA, 20 bp, Artificial sequence
  single strand DNA oligonucleotide
tcttgggtttccagcaattt

SEQ ID NO: 28 - DNA, 564 bp, Lycopersicon esculentum
ggaagaacccttcactttgctcatacccacctcatcaaaatccacctaaatacttctctcactttgctcaatcatctatatagcctaagaattgtcaagattccatttttatagttgttggtgtctgtaatcttggaatttgaacaatttaaagtgaaaagattcaagtttttagaattttctctgcttttgcagttgcagaggtaaagagttgtcaagattccatttttgtagtttattgtgtttgtaatcttgggtttccagcaatttaaagaaaaaaagattcaatctttttaatttatcagtattttggcagctgcagaagtaaagaattggatagcttgaaacccacaaggcaaaagttctagtcttgtttggttaactttccagggagcccaaaattttgtgaaataagagaaatgtgtaccttgagttttatgtatcctaattcacttcttgatggtacctgcaagactgtagctttgggtgatagcaaaccaagatacaataaacagagaagttcttgttttgaccctttgataattggaaattgtactgatcagc

SEQ ID NO: 29 - DNA, 300 bp, Lycopersicon esculentum
tactggaggaacctcaattggaacctcattttccagtttattgatataaagaaaaaggtaatacataatgaactttctttttttgtgtcaaatgtttctttttagcttaacaagtagatatcatgctagtgtccttaaccaaccaagtagtctaagaagagcactgtccagcacatctgattttttttcaaaccctaagtcagctgcaacacgatgagcgcacattactcctgaaaaggctacagctataacaccttgtcctgggaagcaactatcgccaacacaatatagaccatctat

(19) United States
(12) Patent Application Publication
(10) Pub. No.: US 2005/0022269 A1
(43) Pub. Date: Jan. 27, 2005

(54) Polypeptides having carotenoids isomerase catalytic activity, nucleic acids encoding same and uses thereof

(76) Inventors: Joseph Hirschberg, Jerusalem (IL); Tal Isaacson, Arava (IL); Dani Zamir, Gedera (IL)

Correspondence Address: Anthony Castorina;G E Erhlich (1995) Ltd, Suite 207, 2001 Jefferson Davis Highway, Arlington, VA, 22202, US

(21) Appl. No.: 10/483,408

(22) PCT Filed: Jul. 18, 2002
(86) PCT No.: PCT/IL02/00600

Related U.S. Application Data
(60) Provisional application No. 60/306,144, filed on Jul. 19, 2001.

(51) Int. Cl. C12N 15/82; C12N 15/29; A01H 1/00; C12N 9/90

(52) U.S. Cl. 800/298; 536/23.6; 435/320.1; 435/252.3; 800/282; 800/286; 530/370

(57) ABSTRACT

An isolated nucleic acid which comprises a polynucleotide encoding a polypeptide having an amino acid sequence at least 50%, similar to SEQ ID NO: 15 (carotenoid isomerase of tomato (Lycopersicon esculentum)), as determined using the Standard protein-protein BLAST [blastp] software of the NCBI, the polypeptide having carotenoids isomerase catalytic activity, the polypeptide encoded thereby and their uses.

### FIELD AND BACKGROUND OF THE INVENTION

The present invention relates to (i) polypeptides having carotenoids isomerase catalytic activity; (ii) preparations including same; (iii) nucleic acids encoding same; (iv) nucleic acids controlling the expression of same; (v) vectors harboring the nucleic acids; (vi) cells and organisms, inclusive plants, algae, cyanobacteria and naturally non-photosynthetic cells and organisms, genetically modified to express the carotenoids isomerase; and (vii) cells and organisms, inclusive plants, algae and cyanobacteria that naturally express a carotenoids isomerase and are genetically modified to reduce its level of expression.

As part of the light-harvesting antenna, carotenoids can absorb photons and transfer the energy to chlorophyll, thus assisting in the harvesting of light in the range of 450-570 nm [see, Cogdell R J and Frank H A (1987) How carotenoids function in photosynthestic bacteria. Biochim Biophys Acta 895: 63-79; Cogdell R (1988) The function of pigments in chloroplasts. In: Goodwin T W (ed) Plant Pigments, pp 183-255. Academic Press, London; Frank H A, Violette C A, Trautman J K, Shreve A P, Owens T G and Albrecht A C (1991) Carotenoids in photosynthesis: structure and photochemistry. Pure Appl Chem 63: 109-114; Frank H A, Farhoosh R, Decoster B and Christensen R L (1992) Molecular features that control the efficiency of carotenoid-to-chlorophyll energy transfer in photosynthesis. In: Murata N (ed) Research in Photosynthesis, Vol I, pp 125-128. Kluwer, Dordrecht; and, Cogdell R J and Gardiner A T (1993) Functions of carotenoids in photosynthesis. Meth Enzymol 214: 185-193].

Although carotenoids are integral constituents of the protein-pigment complexes of the light-harvesting antennae in photosynthetic organisms, they are also important components of the photosynthetic reaction centers.

Most of the total carotenoids are located in the light harvesting complex II [Bassi R, Pineaw B, Dainese P and Marquartt J (1993) Carotenoid binding proteins of photosystem II. Eur J Biochem 212: 297-302]. The identities of the photosynthetically active carotenoproteins and their precise location in light-harvesting systems are only partially described [Croce R, Weiss S, Bassi R (1999) Carotenoid-binding sites of the major light-harvesting complex II of higher plants. J Biol Chem 274: 29613-29623; Formaggio E, Cinque G, Bassi R (2001) Functional architecture of the major light-harvesting complex from higher plants. J Mol Biol 314: 1157-1166].

Carotenoids in photochemically active chlorophyll-protein complexes of the thermophilic cyanobacterium Synechococcus sp. were investigated by linear dichroism spectroscopy of oriented samples [see, Breton J and Kato S (1987) Orientation of the pigments in photosystem II: low-temperature linear-dichroism study of a core particle and of its chlorophyll-protein subunits isolated from Synechococcus sp. Biochim Biophys Acta 892: 99-107]. These complexes contained mainly a β-carotene pool absorbing around 505 and 470 nm, which is oriented close to the membrane plane. In photochemically inactive chlorophyll-protein complexes, the β-carotene absorbs around 495 and 465 nm, and the molecules are oriented perpendicular to the membrane plane.

Evidence that carotenoids are associated with the cyanobacterial photosystem (PS) II has been described [see, Suzuki R and Fujita Y (1977) Carotenoid photobleaching induced by the action of photosynthetic reaction center II: DCMU sensitivity. Plant Cell Physiol 18: 625-631; and, Newman P J and Sherman L A (1978) Isolation and characterization of photosystem I and II 25 membrane particles from the blue-green alga Synechococcus cedrorum. Biochim Biophys Acta 503: 343-361].

There are two β-carotene molecules in the reaction center core of PS II [see, Ohno T, Satoh K and Katoh S (1986) Chemical composition of purified oxygen-evolving complexes from the thermophilic cyanobacterium Synechococcus sp. Biochim Biophys Acta 852: 1-8; Gounaris K, Chapman D J and Barber J (1989) Isolation and characterization of a D1/D2/cytochrome b-559 complex from Synechocystis PCC6803. Biochim Biophys Acta 973: 296-301; and, Newell R W, van Amerongen H, Barber J and van Grondelle R (1993) Spectroscopic characterization of the reaction center of photosystem II using polarized light: Evidence for β-carotene excitors in PS II reaction centers. Biochim Biophys Acta 1057: 232-238] whose exact function(s) is still obscure [reviewed by Satoh K (1992) Structure and function of PS II reaction center. In: Murata N (ed) Research in Photosynthesis, Vol. II, pp. 3-12. Kluwer, Dordrecht]. It was demonstrated that these two coupled β-carotene molecules protect chlorophyll P680 from photodamage in isolated PS II reaction centers [see, De Las Rivas J, Telfer A and Barber J (1993) 2-coupled β-carotene molecules protect P680 from photodamage in isolated PS II reaction centers. Biochim. Biophys. Acta 1142: 155-164], and this may be related to the protection against degradation of the D1 subunit of PS II [see, Sandmann G (1993) Genes and enzymes involved in the desaturation reactions from phytoene to lycopene. (abstract), 10th International Symposium on Carotenoids, Trondheim CL1-2]. The light-harvesting pigments of a highly purified, oxygen-evolving PS II complex of the thermophilic cyanobacterium Synechococcus sp. consists of 50 chlorophyll α and 7 β-carotene, but no xanthophyll, molecules [see, Ohno T, Satoh K and Katoh S (1986) Chemical composition of purified oxygen-evolving complexes from the thermophilic cyanobacterium Synechococcus sp. Biochim Biophys Acta 852: 1-8]. β-carotene was shown to play a role in the assembly of an active PS II in green algae [see, Humbeck K, Romer S and Senger hours (1989) Evidence for the essential role of carotenoids in the assembly of an active PS II. Planta 179: 242-250].

Isolated complexes of PS I from Phormidium luridum, which contained 40 chlorophylls per P700, contained an average of 1.3 molecules of β-carotene [see, Thomber J P, Alberte R S, Hunter F A , Shiozawa J A and Kan K S (1976) The organization of chlorophyll in the plant photosynthetic unit. Brookhaven Symp Biology 28: 132-148]. In a preparation of PS I particles from Synechococcus sp. strain PCC 6301, which contained 130±5 molecules of antenna chlorophylls per P700, 16 molecules of carotenoids were detected [see, Lundell D J, Glazer A N, Melis A and Malkin R (1985) Characterization of a cyanobacterial photosystem I complex. J Biol Chem 260: 646-654]. A substantial content of β-carotene and the xanthophylls cryptoxanthin and isocryptoxanthin were detected in PS I pigment-protein complexes of the thermophilic cyanobacterium Synechococcus elongatus [see, Coufal J, Hladik J and Sofrova D (1989) The carotenoid content of photosystem 1 pigment-protein complexes of the cyanobacterium Synechococcus elongatus. Photosynthetica 23: 603-616]. A subunit protein-complex structure of PS I from the thermophilic cyanobacterium Synechococcus sp., which consisted of four polypeptides (of 62, 60, 14 and 10 kDa), contained approximately 10 β-carotene molecules per P700 [see, Takahashi Y, Hirota K and Katoh S (1985) Multiple forms of P700-chlorophyll α-protein complexes from Synechococcus sp.: the iron, quinone and carotenoid contents. Photosynth Res 6: 183-192]. This carotenoid is exclusively bound to the large polypeptides which carry the functional and antenna chlorophyll α. The fluorescence excitation spectrum of these complexes suggested that β-carotene serves as an efficient antenna for PS I.

As mentioned, an additional essential function of carotenoids is to protect against photooxidation processes in the photosynthetic apparatus that are caused by the excited triplet state of chlorophyll. Carotenoid molecules with π-electron conjugation of nine or morecarbon-carbon double bonds can absorb triplet-state energy from chlorophyll and thus prevent the formation of harmful singlet-state oxygen radicals. In Synechococcus sp. the triplet state of carotenoids was monitored in closed PS II centers and its rise kinetics of approximately 25 nanoseconds is attributed to energy transfer from chlorophyll triplets in the antenna [see, Schlodder E and Brettel K (1988) Primary charge separation in closed photosystem II with a lifetime of 11 nanoseconds. Flash-absorption spectroscopy with oxygen-evolving photosystem II complexes from Synechococcus. Biochim Biophys Acta 933: 22-34]. It is conceivable that this process, that has a lower yield compared to the yield of radical-pair formation, plays a role in protecting chlorophyll from damage due to over-excitation.

The protective role of carotenoids in vivo has been elucidated through the use of bleaching herbicides such as norflurazon that inhibit carotenoid biosynthesis in all organisms performing oxygenic photosynthesis [reviewed by Sandmann G and Boger P (1989) Inhibition of carotenoid biosynthesis by herbicides. In: Boger P and Sandmann G (Eds.) Target Sites of Herbicide Action, pp 25-44. CRC Press, Boca Raton, Fla.] and by mutants in Chlamydomonas and Arabidopsis [See: Muller-Moule P, Conklin P L, Niyogi K K (2002) Ascorbate deficiency can limit violaxanthin de-epoxidase activity in vivo. Plant Physiol 128: 970-977]. Treatment with norflurazon in the light results in a decrease of both carotenoid and chlorophyll levels, while in the dark, chlorophyll levels are unaffected. Inhibition of photosynthetic efficiency in cells of Oscillatoria agardhii that were treated with the pyridinone herbicide, fluridone, was attributed to a decrease in the relative abundance of myxoxanthophyll, zeaxanthin and β-carotene, which in turn caused photooxidation of chlorophyll molecules [see, Canto de Loura I, Dubacq J P and Thomas J C (1987) The effects of nitrogen deficiency on pigments and lipids of cianobacteria. Plant Physiol 83: 838-843].

It has been demonstrated in plants that zeaxanthin is required to dissipate, in a nonradiative manner, the excess excitation energy of the antenna chlorophyll [see, Demmig-Adams B (1990) Carotenoids and photoprotection in plants: a role for the xanthophyll zeaxanthin. Biochim Biophys Acta 1020: 1-24; and, Demmig-Adams B and Adams W W III (1990) The carotenoid zeaxanthin and high-energy-state quenching of chlorophyll fluorescence. Photosynth Res 25: 187-197]. In algae and plants a light-induced deepoxidation of violaxanthin to yield zeaxanthin, is related to photoprotection processes [reviewed by Demmig-Adams B and Adams W W III (1992) Photoprotection and other responses of plants to high light stress. Ann Rev Plant Physiol Plant Mol Biol 43: 599-626]. The light-induced deepoxidation of violaxanthin and the reverse reaction that takes place in the dark, are known as the “xanthophyll cycle” [see, Demmig-Adams B and Adams W W III (1992) Photoprotection and other responses of plants to high light stress. Ann Rev Plant Physiol Plant Mol Biol 43: 599-626]. Cyanobacterial lichens, that do not contain any zeaxanthin and that probably are incapable of radiationless energy dissipation, are sensitive to high light intensity; algal lichens that contain zeaxanthin are more resistant to high-light stress [see, Demmig-Adams B, Adams W W III, Green T G A, Czygan F C and Lange O L (1990) Differences in the susceptibility to light stress in two lichens forming a phycosymbiodeme, one partner possessing and one lacking the xanthophyll cycle. Oecologia 84: 451-456; Demmig-Adams B and Adams W W III (1993) The xanthophyll cycle, protein turnover, and the high light tolerance of sun-acclimated leaves. Plant Physiol 103: 1413-1420; and, Demmig-Adams B (1990) Carotenoids and photoprotection in plants: a role for the xanthophyll zeaxanthin. Biochim Biophys Acta 1020: 1-24]. In contrast to algae and plants, cyanobacteria do not have a xanthophyll cycle. However, they do contain ample quantities of zeaxanthin and other xanthophylls that can support photoprotection of chlorophyll.

Several other functions have been ascribed to carotenoids. The possibility that carotenoids protect against damaging species generated by near ultra-violet (UV) irradiation is suggested by results describing the accumulation of β-carotene in a UV-resistant mutant of the cyanobacterium Gloeocapsa alpicola [see, Buckley C E and Houghton J A (1976) A study of the effects of near UV radiation on the pigmentation of the blue-green alga Gloeocapsa alpicola. Arch Microbiol 107: 93-97]. This has been demonstrated more elegantly in Escherichia coli cells that produce carotenoids [see, Tuveson R W and Sandmann G (1993) Protection by cloned carotenoid genes expressed in Escherichia coli against phototoxic molecules activated by near-ultraviolet light. Meth Enzymol 214: 323-330]. Due to their ability to quench oxygen radical species, carotenoids are efficient anti-oxidants and thereby protect cells from oxidative damage. This function of carotenoids is important in virtually all organisms [see, Krinsky N I (1989) Antioxidant functions of carotenoids. Free Radical Biol Med 7: 617-635; and, Palozza P and Krinsky N I (1992) Antioxidant effects of carotenoids in vivo and in vitro—an overview. Meth Enzymol 213: 403-420]. Other cellular functions could be affected by carotenoids, even if indirectly. Although carotenoids in cyanobacteria are not the major photoreceptors for phototaxis, an influence of carotenoids on phototactic reactions, that have been observed in Anabaena variabilis, was attributed to the removal of singlet oxygen radicals that may act as signal intermediates in this system [see, Nultsch W and Schuchart hours (1985) A model of the phototactic reaction chain of cyanobacterium Anabaena variabilis. Arch Microbiol 142: 180-184].

In flowers and fruits carotenoids facilitate the attraction of pollinators and dispersal of seeds. This latter aspect is strongly associated with agriculture. The type and degree of pigmentation in fruits and flowers are among the most important traits of many crops. This is mainly since the colors of these products often determine their appeal to the consumers and thus can increase their market worth.

Carotenoids have important commercial uses as coloring agents in the food industry since they are non-toxic [see, Bauernfeind J C (1981) Carotenoids as colorants and vitamin A precursors. Academic Press, London]. The red color of the tomato fruit is provided by lycopene which accumulates during fruit ripening in chromoplasts. Tomato extracts, which contain high content (over 80% dry weight) of lycopene, are commercially produced worldwide for industrial use as food colorant. Furthermore, the flesh, feathers or eggs of fish and birds assume the color of the dietary carotenoid provided, and thus carotenoids are frequently used in dietary additives for poultry and in aquaculture. Certain cyanobacterial species, for example Spirulina sp. [see, Sommer T R, Potts W T and Morrissv N M (1990) Recent progress in processed microalgae in aquaculture. Hydrobiologia 204/205: 435-443], are cultivated in aquaculture for the production of animal and human food supplements. Consequently, the content of carotenoids, primarily of β-carotene, in these cyanobacteria has a major commercial implication in biotechnology.

Most carotenoids are composed of a C₄₀ hydrocarbon backbone, constructed from eight C₅ isoprenoid units and contain a series of conjugated double bonds. Carotenes do not contain oxygen atoms and are either linear or cyclized molecules containing one or two end rings. Xanthophylls are oxygenated derivatives of carotenes. Various glycosilated carotenoids and carotenoid esters have been identified. The C₄₀ backbone can be further extended to give C₄₅ or C₅₀ carotenoids, or shortened yielding apocarotenoids. Some nonphotosynthetic bacteria also synthesize C₃₀ carotenoids. General background on carotenoids can be found in Goodwin T W (1980) The Biochemistry of the Carotenoids, Vol. 1, 2nd Ed. Chapman and Hall, New York; and in Goodwin T W and Britton G (1988) Distribution and analysis of carotenoids. In: Goodwin T W (ed) Plant Pigments, pp 62-132. Academic Press, New York.

More than 640 different naturally-occurring carotenoids have so far been characterized, hence, carotenoids are responsible for most of the various shades of yellow, orange and red found in microorganisms, fungi, algae, plants and animals. Carotenoids are synthesized by all photosynthetic organisms as well as several nonphotosynthetic bacteria and fungi, however they are also widely distributed through feeding throughout the animal kingdom.

Carotenoids are synthesized de novo from isoprenoid precursors only in photosynthetic organisms and some microorganisms, they typically accumulate in protein complexes in the photosynthetic membrane, in the cell membrane and in the cell wall.

In the biosynthesis pathway of β-carotene, four enzymes convert geranylgeranyl pyrophosphate of the central isoprenoid pathway to β-carotene. Carotenoids are produced from the general isoprenoid biosynthetic pathway. While this pathway has been known for several decades, only recently, and mainly through the use of genetics and molecular biology, have some of the molecular mechanisms involved in carotenoids biogenesis, been elucidated. This is due to the fact that most of the enzymes which take part in the conversion of phytoene to carotenes and xanthophylls are labile, membrane-associated proteins that lose activity upon solubilization [see, Beyer P, Weiss G and Kleinig hours (1985) Solubilization and reconstitution of the membrane-bound carotenogenic enzymes from daffodile chromoplasts. Eur J Biochem 153: 341-346; and, Bramley P M (1985) The in vitro biosynthesis of carotenoids. Adv Lipid Res 21: 243-279].

However, solubilization of carotenogenic enzymes from Synechocystis sp. strain PCC 6714 that retain partial activity has been reported [see, Bramley P M and Sandmann G (1987) Solubilization of carotenogenic enzyme of Aphanocapsa. Phytochem 26: 1935-1939].

There is no genuine in vitro system for carotenoid biosynthesis which enables a direct essay of enzymatic activities. A cell-free carotenogenic system has been developed [see, Clarke I E, Sandmann G, Bramley P M and Boger P (1982) Carotene biosynthesis with isolated photosynthetic membranes. FEBS Lett 140: 203-206] and adapted for cyanobacteria [see, Sandmann G and Bramley P M (1985) Carotenoid biosynthesis by Aphanocapsa homogenates coupled to a phytoene-generating system from Phycomyces blakesleeanus. Planta 164: 259-263; and, Bramley P M and Sandmann G (1985) In vitro and in vivo biosynthesis of xanthophylls by the cyanobacterium Aphanocapsa. Phytochem 24: 2919-2922]. Reconstitution of phytoene desaturase from Synechococcus sp. strain PCC 7942 in liposomes was achieved following purification of the polypeptide, that had been expressed in Escherichia coli [see, Fraser P D, Linden hours and Sandmann G (1993) Purification and reactivation of recombinant Synechococcus phytoene desaturase from an overexpressing strain of Escherichia coli. Biochem J 291: 687-692].

Carotenoids are synthesized from isoprenoid precursors. The central pathway of isoprenoid biosynthesis may be viewed as beginning with the conversion of acetyl-CoA to mevalonic acid. D³-isopentenyl pyrophosphate (IPP), a C₅ molecule, is formed from mevalonate and is the building block for all long-chain isoprenoids. Following isomerization of IPP to dimethylallyl pyrophosphate (DMAPP), three additional molecules of IPP are combined to yield the C₂₀ molecule, geranylgeranyl pyrophosphate (GGPP). These 1′-4 condensation reactions are catalyzed by prenyl transferases [see, Kleinig hours (1989) The role of plastids in isoprenoid biosynthesis. Ann Rev Plant Physiol Plant Mol Biol 40: 39-59]. There is evidence in plants that the same enzyme, GGPP synthase, carries out all the reactions from DMAPP to GGPP [see, Dogbo O and Camara B (1987) Purification of isopentenyl pyrophosphate isomerase and geranylgeranyl pyrophosphate synthase from Capsicum chromoplasts by affinity chromatography. Biochim Biophys Acta 920: 140-148; and, Laferriere A and Beyer P (1991) Purification of geranylgeranyl diphosphate synthase from Sinapis alba etioplasts. Biochim Biophys Acta 216: 156-163].

The first step that is specific for carotenoid biosynthesis is the head-to-head condensation of two molecules of GGPP to produce prephytoene pyrophosphate (PPPP). Following removal of the pyrophosphate, GGPP is converted to 15-cis-phytoene, a colorless C₄₀ hydrocarbon molecule. This two-step reaction is catalyzed by the soluble enzyme, phytoene synthase, an enzyme encoded by a single gene (crtB), in both cyanobacteria and plants [see, Chamovitz D, Misawa N, Sandmann G and Hirschberg J (1992) Molecular cloning and expression in Escherichia coli of a cyanobacterial gene coding for phytoene synthase, a carotenoid biosynthesis enzyme. FEBS Lett 296: 305-310; Ray J A, Bird C R, Maunders M, Grierson D and Schuch W (1987) Sequence of pTOM5, a ripening related cDNA from tomato. Nucl Acids Res 15: 10587-10588; Camara B (1993) Plant phytoene synthase complex—component 3 enzymes, immunology, and biogenesis. Meth Enzymol 214: 352-365]. All the subsequent steps in the pathway occur in membranes. Four desaturation (dehydrogenation) reactions convert phytoene to lycopene via phytofluene, ζ-carotene, and neurosporene. Each desaturation increases the number of conjugated double bonds by two such that the number of conjugated double bonds increases from three in phytoene to eleven in lycopene.

Relatively little is known about the molecular mechanism of the enzymatic dehydrogenation of phytoene [see, Jones B L and Porter J W (1986) Biosynthesis of carotenes in higher plants. CRC Crit Rev Plant Sci 3: 295-324; and, Beyer P, Mayer M and Kleinig hours (1989) Molecular oxygen and the state of geometric iosomerism of intermediates are essential in the carotene desaturation and cyclization reactions in daffodil chromoplasts. Eur J Biochem 184: 141-150]. It has been established that in cyanobacteria, algae and plants the first two desaturations, from 15-cis-phytoene to ζ-carotene, are catalyzed by a single membrane-bound enzyme, phytoene desaturase [see, Jones B L and Porter J W (1986) Biosynthesis of carotenes in higher plants. CRC Crit Rev Plant Sci 3: 295-324; and, Beyer P, Mayer M and Kleinig hours (1989) Molecular oxygen and the state of geometric iosomerism of intermediates are essential in the carotene desaturation and cyclization reactions in daffodil chromoplasts. Eur J Biochem 184: 141-150]. Since the ζ-carotene product is mostly in the all-trans configuration, a cis-trans isomerization is presumed at this desaturation step. The primary structure of the phytoene desaturase polypeptide in cyanobacteria is conserved (over 65% identical residues) with that of algae and plants [see, Pecker I, Chamovitz D, Linden H, Sandmann G and Hirschberg J (1992) A single polypeptide catalyzing the conversion of phytoene to ζ-carotene is transcriptionally regulated during tomato fruit ripening. Proc Natl Acad Sci USA 89: 4962-4966; Pecker I, Chamovitz D, Mann V, Sandmann G, Boger P and Hirschberg J (1993) Molecular characterization of carotenoid biosynthesis in plants: the phytoene desaturase gene in tomato. In: Murata N (ed) Research in Photosynthesis, Vol III, pp 11-18. Kluwer, Dordrectht]. Moreover, the same inhibitors block phytoene desaturase in the two systems [see, Sandmann G and Boger P (1989) Inhibition of carotenoid biosynthesis by herbicides. In: Boger P and Sandmann G (eds) Target Sites of Herbicide Action, pp 25-44. CRC Press, Boca Raton, Fla.]. Consequently, it is very likely that the enzymes catalyzing the desaturation of phytoene and phytofluene in cyanobacteria and plants have similar biochemical and molecular properties, that are distinct from those of phytoene desaturases in other microorganisms. One such a difference is that phytoene desaturases from Rhodobacter capsulatus, Erwinia sp. or fungi convert phytoene to neurosporene, lycopene, or 3,4-dehydrolycopene, respectively.

Desaturation of phytoene in daffodil chromoplasts [see, Beyer P, Mayer M and Kleinig hours (1989) Molecular oxygen and the state of geometric iosomerism of intermediates are essential in the carotene desaturation and cyclization reactions in daffodil chromoplasts. Eur J Biochem 184: 141-150], as well as in a cell free system of Synechococcus sp. strain PCC 7942 [see, Sandmann G and Kowalczyk S (1989) In vitro carotenogenesis and characterization of the phytoene desaturase reaction in Anacystis. Biochem Biophys Res Com 163: 916-921], is dependent on molecular oxygen as a possible final electron acceptor, although oxygen is not directly involved in this reaction. A mechanism of dehydrogenase-electron transferase was supported in cyanobacteria over dehydrogenation mechanism of dehydrogenase-monooxygenase [see, Sandmann G and Kowalczyk S (1989) In vitro carotenogenesis and characterization of the phytoene desaturase reaction in Anacystis. Biochem Biophys Com 163: 916-921]. A conserved FAD-binding motif exists in all phytoene desaturases whose primary structures have been analyzed [see, Pecker I, Chamovitz D, Linden H, Sandmann G and Hirschberg J (1992) A single polypeptide catalyzing the conversion of phytoene to ζ-carotene is transcriptionally regulated during tomato fruit ripening. Proc Natl Acad Sci USA 89: 4962-4966; Pecker I, Chamovitz D, Mann V, Sandmann G, Boger P and Hirschberg J (1993) Molecular characterization of carotenoid biosynthesis in plants: the phytoene desaturase gene in tomato. In: Murata N (ed) Research in Photosynthesis, Vol III, pp 11-18. Kluwer, Dordrectht]. The phytoene desaturase enzyme in pepper was shown to contain a protein-bound FAD [see, Hugueney P, Romer S, Kuntz M and Camara B (1992) Characterization and molecular cloning of a flavoprotein catalyzing the synthesis of phytofluene and ζ-carotene in Capsicum chromoplasts. Eur J Biochem 209: 399-407]. Since phytoene desaturase is located in the membrane, an additional, soluble redox component is predicted. This hypothetical component could employ NAD(P)⁺, as suggested [see, Mayer M P, Nievelstein V and Beyer P (1992) Purification and characterization of a NADPH dependent oxidoreductase from chromoplasts of Narcissus pseudonarcissus—a redox-mediator possibly involved in carotene desaturation. Plant Physiol Biochem 30: 389-398] or another electron and hydrogen carrier, such as a quinone. The cellular location of phytoene desaturase in Synechocystis sp. strain PCC 6714 and Anabaena variabilis strain ATCC 29413 was determined with specific antibodies to be mainly (85%) in the photosynthetic thylakoid membranes [see, Serrano A, Gimenez P, Schmidt A and Sandmann G (1990) Immunocytochemical localization and functional determination of phytoene desaturase in photoautotrophic prokaryotes. J Gen Microbiol 136: 2465-2469].

In cyanobacteria, algae and plants ζ-carotene is converted to lycopene via neurosporene. Very little is known about the enzymatic mechanism, which is predicted to be carried out by a single enzyme [see, Linden H, Vioque A and Sandmann G (1993) Isolation of a carotenoid biosynthesis gene coding for ζ-carotene desaturase from Anabaena PCC 7120 by heterologous complementation. FEMS Microbiol Lett 106: 99-104]. The deduced amino acid sequence of ζ-carotene desaturase in Anabaena sp. strain PCC 7120 contains a dinucleotide-binding motif that is similar to the one found in phytoene desaturase.

Two cyclization reactions convert lycopene to β-carotene. Evidence has been obtained that in Synechococcus sp. strain PCC 7942 [see, Cunningham F X Jr, Chamovitz D, Misawa N, Gantt E and Hirschberg J (1993) Cloning and functional expression in Escherichia coli of a cyanobacterial gene for lycopene cyclase, the enzyme that catalyzes the biosynthesis of β-carotene. FEBS Lett 328: 130-138], as well as in plants [see, Camara B and Dogbo O (1986) Demonstration and solubilization of lycopene cyclase from Capsicum chromoplast membranes. Plant Physiol 80: 172-184], these two cyclizations are catalyzed by a single enzyme, lycopene cyclase. This membrane-bound enzyme is inhibited by the triethylamine compounds, CPTA and MPTA [see, Sandmann G and Boger P (1989) Inhibition of carotenoid biosynthesis by herbicides. In: Boger P and Sandmann G (eds) Target Sites of Herbicide Action, pp 25-44. CRC Press, Boca Raton, Fla.]. Cyanobacteria carry out only the β-cyclization and therefore do not contain ε-carotene, δ-carotene and α-carotene and their oxygenated derivatives. The β-ring is formed through the formation of a “carbonium ion” intermediate when the C-1,2 double bond at the end of the linear lycopene molecule is folded into the position of the C-5,6 double bond, followed by a loss of a proton from C-6. No cyclic carotene has been reported in which the 7,8 bond is not a double bond. Therefore, full desaturation as in lycopene, or desaturation of at least half-molecule as in neurosporene, is essential for the reaction. Cyclization of lycopene involves a dehydrogenation reaction that does not require oxygen. The cofactor for this reaction is unknown. A dinucleotide-binding domain was found in the lycopene cyclase polypeptide of Synechococcus sp. strain PCC 7942, implicating NAD(P) or FAD as coenzymes with lycopene cyclase.

The addition of various oxygen-containing side groups, such as hydroxy-, methoxy-, oxo-, epoxy-, aldehyde or carboxylic acid moieties, form the various xanthophyll species. Little is known about the formation of xanthophylls. Hydroxylation of β-carotene requires molecular oxygen in a mixed-function oxidase reaction.

Clusters of genes encoding the enzymes for the entire pathway have been cloned from the purple photosynthetic bacterium Rhodobacter capsulatus [see, Armstrong G A, Alberti M, Leach F and Hearst J E (1989) Nucleotide sequence, organization, and nature of the protein products of the carotenoid biosynthesis gene cluster of Rhodobacter capsulatus. Mol Gen Genet 216: 254-268] and from the nonphotosynthetic bacteria Erwinia herbicola [see, Sandmann G, Woods W S and Tuveson R W (1990) Identification of carotenoids in Erwinia herbicola and in transformed Escherichia coli strain. FEMS Microbiol Lett 71: 77-82; Hundle B S, Beyer P, Kleinig H, Englert hours and Hearst J E (1991) Carotenoids of Erwinia herbicola and an Escherichia coli HB101 strain carrying the Erwinia herbicola carotenoid gene cluster. Photochem Photobiol 54: 89-93; and, Schnurr G, Schmidt A and Sandmann G (1991) Mapping of a carotenogenic gene cluster from Erwinia herbicola and functional identification of six genes. FEMS Microbiol Lett 78: 157-162] and Erwinia uredovora [see, Misawa N, Nakagawa M, Kobayashi K, Yamano S, Izawa I, Nakamura K and Harashima K (1990) Elucidation of the Erwinia uredovora carotenoid biosynthetic pathway by functional analysis of gene products in Escherichia coli. J Bacteriol 172: 6704-6712]. Two genes, al-3 for GGPP synthase [see, Nelson M A, Morelli G, Carattoli A, Romano N and Macino G (1989) Molecular cloning of a Neurospora crassa carotenoid biosynthetic gene (albino-3) regulated by blue light and the products of the white collar genes. Mol Cell Biol 9: 1271-1276; and, Carattoli A, Romano N, Ballario P, Morelli G and Macino G (1991) The Neurospora crassa carotenoid biosynthetic gene (albino 3). J Biol Chem 266: 5854-5859] and al-1 for phytoene desaturase [see, Schmidhauser T J, Lauter F R, Russo V E A and Yanofsky C (1990) Cloning sequencing and photoregulation of al-1, a carotenoid biosynthetic gene of Neurospora crassa. Mol Cell Biol 10: 5064-5070] have been cloned from the fungus Neurospora crassa. However, attempts at using these genes as heterologous molecular probes to clone the corresponding genes from cyanobacteria or plants were unsuccessful due to lack of sufficient sequence similarity.

The first “plant-type” genes for carotenoid synthesis enzyme were cloned from cyanobacteria using a molecular-genetics approach. In the first step towards cloning the gene for phytoene desaturase, a number of mutants that are resistant to the phytoene-desaturase-specific inhibitor, norflurazon, were isolated in Synechococcus sp. strain PCC 7942 [see, Linden H, Sandmann G, Chamovitz D, Hirschberg J and Boger P (1990) Biochemical characterization of Synechococcus mutants selected against the bleaching herbicide norflurazon. Pestic Biochem Physiol 36: 46-51]. The gene conferring norflurazon-resistance was then cloned by transforming the wild-type strain to herbicide resistance [see, Chamovitz D, Pecker I and Hirschberg J (1991) The molecular basis of resistance to the herbicide norflurazon. Plant Mol Biol 16: 967-974; Chamovitz D, Pecker I, Sandmann G, Boger P and Hirschberg J (1990) Cloning a gene for norflurazon resistance in cyanobacteria. Z Naturforsch 45c: 482-486]. Several lines of evidence indicated that the cloned gene, formerly called pds and now named crtP, codes for phytoene desaturase. The most definitive one was the functional expression of phytoene desaturase activity in transformed Escherichia coli cells [see, Linden H, Misawa N, Chamovitz D, Pecker I, Hirschberg J and Sandmann G (1991) Functional complementation in Escherichia coli of different phytoene desaturase genes and analysis of accumulated carotenes. Z Naturforsch 46c: 1045-1051; and, Pecker I, Chamovitz D, Linden H, Sandmann G and Hirschberg J (1992) A single polypeptide catalyzing the conversion of phytoene to ζ-carotene is transcriptionally regulated during tomato fruit ripening. Proc Natl Acad Sci USA 89: 4962-4966]. The crtP gene was also cloned from Synechocystis sp. strain PCC 6803 by similar methods [see, Martinez-Ferez I M and Vioque A (1992) Nucleotide sequence of the phytoene desaturase gene from Synechocystis sp. PCC 6803 and characterization of a new mutation which confers resistance to the herbicide norflurazon. Plant Mol Biol 18: 981-983].

The cyanobacterial crtP gene was subsequently used as a molecular probe for cloning the homologous gene from an alga [see, Pecker I, Chamovitz D, Mann V, Sandmann G, Boger P and Hirschberg J (1993) Molecular characterization of carotenoid biosynthesis in plants: the phytoene desaturase gene in tomato. In: Murata N (ed) Research in Photosynthesis, Vol III, pp 11-18. Kluwer, Dordrectht] and higher plants [see, Bartley G E, Viitanen P V, Pecker I, Chamovitz D, Hirschberg J and Scolnik P A (1991) Molecular cloning and expression in photosynthetic bacteria of a soybean cDNA coding for phytoene desaturase, an enzyme of the carotenoid biosynthesis pathway. Proc Natl Acad Sci USA 88: 6532-6536; and, Pecker I, Chamovitz D, Linden H, Sandmann G and Hirschberg J (1992) A single polypeptide catalyzing the conversion of phytoene to ζ-carotene is transcriptionally regulated during tomato fruit ripening. Proc Natl Acad Sci USA 89: 4962-4966]. The phytoene desaturases in Synechococcus sp. strain PCC 7942 and Synechocystis sp. strain PCC 6803 consist of 474 and 467 amino acid residues, respectively, whose sequences are highly conserved (74% identities and 86% similarities). The calculated molecular mass is 51 kDa and, although it is slightly hydrophobic (hydropathy index −0.2), it does not include a hydrophobic region which is long enough to span a lipid bilayer membrane. The primary structure of the cyanobacterial phytoene desaturase is highly conserved with the enzyme from the green alga Dunalliela bardawil (61% identical and 81% similar; [see, Pecker I, Chamovitz D, Mann V, Sandmann G, Boger P and Hirschberg J (1993) Molecular characterization of carotenoid biosynthesis in plants: the phytoene desaturase gene in tomato. In: Murata N (ed) Research in Photosynthesis, Vol III, pp 11-18. Kluwer, Dordrectht]) and from tomato [see, Pecker I, Chamovitz D, Linden H, Sandmann G and Hirschberg J (1992) A single polypeptide catalyzing the conversion of phytoene to ζ-carotene is transcriptionally regulated during tomato fruit ripening. Proc Natl Acad Sci USA 89: 4962-4966], pepper [see, Hugueney P, Romer S, Kuntz M and Camara B (1992) Characterization and molecular cloning of a flavoprotein catalyzing the synthesis of phytofluene and ζ-carotene in Capsicum chromoplasts. Eur J Biochem 209: 399-407] and soybean [see, Bartley G E, Viitanen P V, Pecker I, Chamovitz D, Hirschberg J and Scolnik P A (1991) Molecular cloning and expression in photosynthetic bacteria of a soybean cDNA coding for phytoene desaturase, an enzyme of the carotenoid biosynthesis pathway. Proc Natl Acad Sci USA 88: 6532-6536] (62-65% identical and ˜79% similar; [see, Chamovitz D (1993) Molecular analysis of the early steps of carotenoid biosynthesis in cyanobacteria: Phytoene synthase and phytoene desaturase. Ph.D. Thesis, The Hebrew University of Jerusalem]). The eukaryotic phytoene desaturase polypeptides are larger (64 kDa); however, they are processed during import into the plastids to mature forms whose sizes are comparable to those of the cyanobacterial enzymes.

There is a high degree of structural similarity in carotenoid enzymes of Rhodobacter capsulatus, Erwinia sp. and Neurospora crassa [reviewed in Armstrong G A, Hundle B S and Hearst J E (1993) Evolutionary conservation and structural similarities of carotenoid biosynthesis gene products from photosynthetic and nonphotosynthetic organisms. Meth Enzymol 214: 297-311], including in the crtI gene-product, phytoene desaturase. As indicated above, a high degree of conservation of the primary structure of phytoene desaturases also exists among oxygenic photosynthetic organisms. However, there is little sequence similarity, except for the FAD binding sequences at the amino termini, between the “plant-type” crtP gene products and the “bacterial-type” phytoene desaturases (crtI gene products; 19-23% identities and 42-47% similarities). It has been hypothesized that crtP and crtI are not derived from the same ancestral gene and that they originated independently through convergent evolution [see, Pecker I, Chamovitz D, Linden H, Sandmann G and Hirschberg J (1992) A single polypeptide catalyzing the conversion of phytoene to ζ-carotene is transcriptionally regulated during tomato fruit ripening. Proc Natl Acad Sci USA 89: 4962-4966]. This hypothesis is supported by the different dehydrogenation sequences that are catalyzed by the two types of enzymes and by their different sensitivities to inhibitors.

Although not as definite as in the case of phytoene desaturase, a similar distinction between cyanobacteria and plants on the one hand and other microorganisms is also seen in the structure of phytoene synthase. The crtB gene (formerly psy) encoding phytoene synthase was identified in the genome of Synechococcus sp. strain PCC 7942 adjacent to crtP and within the same operon [see, Bartley G E, Viitanen P V, Pecker I, Chamovitz D, Hirschberg J and Scolnik P A (1991) Molecular cloning and expression in photosynthetic bacteria of a soybean cDNA coding for phytoene desaturase, an enzyme of the carotenoid biosynthesis pathway. Proc Natl Acad Sci USA 88: 6532-6536]. This gene encodes a 36-kDa polypeptide of 307 amino acids with a hydrophobic index of −0.4. The deduced amino acid sequence of the cyanobacterial phytoene synthase is highly conserved with the tomato phytoene synthase (57% identical and 70% similar; Ray J A, Bird C R, Maunders M, Grierson D and Schuch W (1987) Sequence of pTOM5, a ripening related cDNA from tomato. Nucl Acids Res 15: 10587-10588]) but is less highly conserved with the crtB sequences from other bacteria (29-32% identical and 48-50% similar with ten gaps in the alignment). Both types of enzymes contain two conserved sequence motifs also found in prenyl transferases from diverse organisms [see, Bartley G E , Viitanen P V, Pecker I, Chamovitz D, Hirschberg J and Scolnik P A (1991) Molecular cloning and expression in photosynthetic bacteria of a soybean cDNA coding for phytoene desaturase, an enzyme of the carotenoid biosynthesis pathway. Proc Natl Acad Sci USA 88: 6532-6536; Carattoli A, Romano N, Ballario P, Morelli G and Macino G (1991) The Neurospora crassa carotenoid biosynthetic gene (albino 3). J Biol Chem 266: 5854-5859; Armstrong G A, Hundle B S and Hearst J E (1993) Evolutionary conservation and structural similarities of carotenoid biosynthesis gene products from photosynthetic and nonphotosynthetic organisms. Meth Enzymol 214: 297-311; Math S K, Hearst J E and Poulter C D (1992) The crtE gene in Erwinia herbicola encodes geranylgeranyl diphosphate synthase. Proc Natl Acad Sci USA 89: 6761-6764; and, Chamovitz D (1993) Molecular analysis of the early steps of carotenoid biosynthesis in cyanobacteria: Phytoene synthase and phytoene desaturase. Ph.D. Thesis, The Hebrew University of Jerusalem]. It is conceivable that these regions in the polypeptide are involved in the binding and/or removal of the pyrophosphate during the condensation of two GGPP molecules.

The crtQ gene encoding ζ-carotene desaturase (formerly zds) was cloned from Anabaena sp. strain PCC 7120 by screening an expression library of cyanobacterial genomic DNA in cells of Escherichia coli carrying the Erwinia sp. crtB and crtE genes and the cyanobacterial crtP gene [see, Linden H, Vioque A and Sandmann G (1993) Isolation of a carotenoid biosynthesis gene coding for ζ-carotene desaturase from Anabaena PCC 7120 by heterologous complementation. FEMS Microbiol Lett 106: 99-104]. Since these Escherichia coli cells produce ζ-carotene, brownish-red pigmented colonies that produced lycopene could be identified on the yellowish background of cells producing ζ-carotene. The predicted ζ-carotene desaturase from Anabaena sp. strain PCC 7120 is a 56-kDa polypeptide which consists of 499 amino acid residues. Surprisingly, its primary structure is not conserved with the “plant-type” (crtP gene product) phytoene desaturases, but it has considerable sequence similarity to the bacterial-type enzyme (crtI gene product) [see, Sandmann G (1993) Genes and enzymes involved in the desaturation reactions from phytoene to lycopene. (abstract), 10th International Symposium on Carotenoids, Trondheim CL1-2]. It is possible that the cyanobacterial crtQ gene and crtI gene of other microorganisms originated in evolution from a common ancestor.

The crtL gene for lycopene cyclase (formerly lcy) was cloned from Synechococcus sp. strain PCC 7942 utilizing essentially the same cloning strategy as for crtP. By using an inhibitor of lycopene cyclase, 2-(4-methylphenoxy)-triethylamine hydrochloride (MPTA), the gene was isolated by transformation of the wild-type to herbicide-resistance [see, Cunningham F X Jr, Chamovitz D, Misawa N, Gantt E and Hirschberg J (1993) Cloning and functional expression in Escherichia coli of a cyanobacterial gene for lycopene cyclase, the enzyme that catalyzes the biosynthesis of β-carotene. FEBS Lett 328: 130-138]. Lycopene cyclase is the product of a single gene product and catalyzes the double cyclization reaction of lycopene to β-carotene. The crtL gene product in Synechococcus sp. strain PCC 7942 is a 46-kDa polypeptide of 411 amino acid residues. It has no sequence similarity to the crtY gene product (lycopene cyclase) from Erwinia uredovora or Erwinia herbicola.

The gene for β-carotene hydroxylase (crtZ) and zeaxanthin glycosilase (crtX) have been cloned from Erwinia herbicola [see, Hundle B, Alberti M, Nievelstein V, Beyer P, Kleinig H, Armstrong G A, Burke D H and Hearst J E (1994) Functional assignment of Erwinia herbicola Eho10 carotenoid genes expressed in Escherichia coli. Mol Gen Genet 254: 406-416; Hundle B S, Obrien D A, Alberti M, Beyer P and Hearst J E (1992) Functional expression of zeaxanthin glucosyltransferase from Erwinia herbicola and a proposed diphosphate binding site. Proc Natl Acad Sci USA 89: 9321-9325] and from Erwinia uredovora [see, Misawa N, Nakagawa M, Kobayashi K, Yamano S, Izawa I, Nakamura K and Harashima K (1990) Elucidation of the Erwinia uredovora carotenoid biosynthetic pathway by functional analysis of gene products in Escherichia coli. J Bacteriol 172: 6704-6712].

The gene encoding beta-C-4-oxygenase from H. pluvialis is described in U.S. Pat. No. 5,965,795. The gene encoding lycopene cyclase from tomato is described in U.S. Pat. No. 6,252,141.

Like all other isoprenoids carotenoids are built from the 5-carbon compound isopentenyl diphosphate (IPP). IPP in plastids is produced in the “DOXP pathway” from pyruvate and glyceraldehyde-3-phosphate [Lichtenthaler H K, Schwender J, Disch A, Rohmer M: Biosynthesis of isoprenoids in higher plant chloroplasts proceeds via a mevalonate-independent pathway. FEBS Lett. 1997, 400:271-274; Lichtenthaler H K, Rohmer M, Schwender J: Two independent biochemical pathways for isopentenyl diphosphate and isoprenoid biosynthesis in higher plants. Physiol.Plant. 1997, 101:643-652]. The first enzyme in the pathway is 1-deoxyxylulose 5-phosphate (DOXP) synthase (DXS), whose gene was cloned from pepper C. annuum [Bouvier F, d'Harlingue A, Suire C, Backhaus R A, Camara B: Dedicated roles of plastid transketolases during the early onset of isoprenoid biogenesis in pepper fruits. Plant Physiol. 1998, 117:1423-1431] Mentha piperita [Lange B M, Croteau R: Isoprenoid biosynthesis via a mevalonate-independent pathway in plants: cloning and heterologous expression of 1-deoxy-D-xylulose-5-phosphate reductoisomerase from peppermint. Arch.Biochem.Biophys. 1999, 365:170-174], tomato (L. esculentum) [Lois L M, Rodriguez-Concepcion M, Gallego F, Campos N, Boronat A: Carotenoid biosynthesis during tomato fruit development: regulatory role of 1-deoxy-D-xylulose 5-phosphate synthase. Plant J. 2000, 22:503-513] and Arabidopsis thaliana [Araki N, Kusumi K, Masamoto K, Niwa Y, Iba K: Temperature-sensitive Arabidopsis mutant defective in 1-deoxy-D-xylulose 5-phosphate synthase within the plastid non-mevalonate pathway of isoprenoid biosynthesis. Physiol.Plant. 2000, 108:19-24]. In the temperature-sensitive mutant of Arabidopsis, chs5, DXS is impaired. At the restrictive temperature chlorotic leaves develop in young leaf tissues, but not in mature leaves, indicating that DXS functions preferentially at an early stage of leaf development [Araki N, Kusumi K, Masamoto K, Niwa Y, Iba K: Temperature-sensitive Arabidopsis mutant defective in 1-deoxy-D-xylulose 5-phosphate synthase within the plastid non-mevalonate pathway of isoprenoid biosynthesis. Physiol.Plant. 2000, 108:19-24]. It has been suggested that DXS could potentially be a regulatory step in carotenoid biosynthesis during early fruit ripening in tomato [Lois L M, Rodriguez-Concepcion M, Gallego F, Campos N, Boronat A: Carotenoid biosynthesis during tomato fruit development: regulatory role of 1-deoxy-D-xylulose 5-phosphate synthase. Plant J. 2000, 22:503-513]. DOXP is converted to 2C-methyl-D-erythritol 2,4-cyclodiphosphate via 2C-methyl-D-erythritol 4-phosphate, 4-diphosphocytidyl-2C-methyl-D-erythritol and 4-diphosphocytidyl-2C-methyl-D-erythritol 2-phosphate. These steps are catalyzed by the enzymes DXR, ISPD (ygbP), ISPE and ISPF, respectively (reviewed in: [Eisenreich W, Rohdich F, Bacher A: Deoxyxylulose phosphate pathway to terpenoids. Trends Plant Sci. 2001, 6:78-84]). The gene Dxr was cloned from A. thaliana [Schwender J, Muller C, Zeidler J, Lichtenthaler H K: Cloning and heterologous expression of a cDNA encoding 1-deoxy-D-xylulose-5-phosphate reductoisomerase of Arabidopsis thaliana. FEBS Lett. 1999, 455: 140-144] and M. piperita [Lange B M, Croteau R: Isoprenoid biosynthesis via a mevalonate-independent pathway in plants: cloning and heterologous expression of 1-deoxy-D-xylulose-5-phosphate reductoisomerase from peppermint. Arch.Biochem.Biophys. 1999, 365:170-174]; The gene IspD was cloned from A. thaliana [Rohdich F, Wungsintaweekul J, Eisenreich W, Richter G, Schuhr C A, Hecht S, Zenk M H, Bacher A: Biosynthesis of terpenoids: 4-Diphosphocytidyl-2C-methyl-D-erythritol synthase of Arabidopsis thaliana. Proc.Natl.Acad.Sci.U.S.A. 2000, 97:6451-6456] and the gene ispE was cloned from M. piperita [Lange B M, Croteau R: Isoprenoid biosynthesis via a mevalonate-independent pathway in plants: cloning and heterologous expression of 1-deoxy-D-xylulose-5-phosphate reductoisomerase from peppermint. Arch.Biochem.Biophys. 1999, 365:170-174] and tomato [Rohdich F, Wungsintaweekul J, Luttgen H, Fischer M, Eisenreich W, Schuhr C A, Fellermeier M, Schramek N, Zenk M H, Bacher A: Biosynthesis of terpenoids: 4-diphosphocytidyl-2-C-methyl-D-erythritol kinase from tomato. Proc.Natl.Acad.Sci.U.S.A. 2000, 97:8251-8256]. An enzyme encoded by the gene LytB, which was recently cloned from Adonis aestivalis, has been hypothesized to catalyze a subsequent reaction that affects the ratio of IPP to dimethylallyl diphosphate (DMAPP) [Cunningham F X Jr, Lafond T P, Gantt E: Evidence of a role for LytB in the nonmevalonate pathway of isoprenoid biosynthesis. J.Bacteriol. 2000, 182:5841-5848]. IPP is isomerized to DMAPP by the enzyme IPP isomerase (encoded by the gene Ipi). There are two Ipi genes in plants and one of them is predicted to be targeted to the plastids (reviewed in: [Cunningham F X Jr, Gantt E: Genes and enzymes of carotenoid biosynthesis in plants. Ann.Rev.Plant Physiol.Plant Mol.Biol. 1998, 49:557-583]). Sequential addition of 3 IPP molecules to DMADP gives the 20-carbon molecule geranylgeranyl diphosphate (GGPP), which is catalyzed by a single enzyme GGPP synthase (GGPS). The genome of Arabidopsis contains a family of 12 genes that are similar to Ggps [Kaul S, Analysis of the genome sequence of the flowering plant Arabidopsis thaliana. Nature 2000, 408:796-815]. It is not yet clear how many of them are involved in the formation of GGPP in the plastids. Five Ggps genes were shown to be expressed in different tissues during plant development [Okada K, Saito T, Nakagawa T, Kawamukai M, Kamiya Y: Five geranylgeranyl diphosphate synthases expressed in different organs are localized into three subcellular compartments in Arabidopsis. Plant Physiol. 2000, 122:1045-1056].

The first committed step in the carotenoid pathway is the condensation of two GGPP molecules to produce 15-cis phytoene, catalyzed by a membrane-associated enzyme phytoene synthase (PSY) (FIG. 1) [Camara B: Plant phytoene synthase complex—component enzymes, immunology, and biogenesis. Methods Enzymol. 1993, 214:352-365]. PSY shares amino acid sequence similarity with GGPP synthase and other prenyl-transferases. Partial purification of PSY from tomato indicated that the enzyme is associated with the isoprenoid biosynthesis enzymes IPI and GGPPS in a protein complex that is larger than 200 kDa [Fraser P D, Schuch W, Bramley P M: Phytoene synthase from tomato (Lycopersicon esculentum) chloroplasts—partial purification and biochemical properties. Planta 2000, 211:361-369]. In tomato there are two genes for PSY, Psy-1, which encodes a fruit and flower-specific isoform, and Psy-2, which encodes an isoform that predominates in green tissues [Bartley G E , Scolnik P A: cDNA cloning, expression during development, and genome mapping of PSY2, a second tomato gene encoding phytoene synthase. J.Biol.Chem. 1993, 268:25718-25721; Fraser P D, Kiano J W, Truesdale M R, Schuch W, Bramley P M: Phytoene synthase-2 enzyme activity in tomato does not contribute to carotenoid synthesis in ripening fruit. Plant Mol.Biol. 1999, 40:687-698]. PSY is a rate limiting step in ripening tomato fruits [Fraser P D, Truesdale M R, Bird C R, Schuch W, Bramley P M: Carotenoid biosynthesis during tomato fruit development. Plant Physiol. 1994, 105:405-413], in canola (Brassica napus) seeds [Shewmaker C K, Sheehy J A, Daley M, Colburn S, Ke D Y: Seed-specific overexpression of phytoene synthase: increase in carotenoids and other metabolic effects. Plant J. 1999, 20:401-412] and in marigold flowers [Moehs C P, Tian L, Osteryoung K W, Dellapenna D: Analysis of carotenoids biosynthetic gene expression during marigold petal development. Plant Mol.Biol. 2001, 45:281-293]. This feature would make PSY suitable to be a key regulator of carotenogenesis.

Two structurally and functionally similar enzymes, phytoene desaturase (PDS) and ζ-carotene desaturase (ZDS), convert phytoene to lycopene via ζ-carotene. These FAD-containing enzymes catalyze each two symmetric dehydrogenation reactions that require plastoquinone [Mayer M P, Nievelstein V, Beyer P: Purification and characterization of a NADPH dependent oxidoreductase from chromoplasts of Narcissus pseudonarcissus—A redox-mediator possibly involved in carotene desaturation. Plant.Physiol.Biochem. 1992, 30:389-398; Norris S R, Barrette T R, Dellapenna D: Genetic dissection of carotenoid synthesis in Arabidopsis defines plastoquinone as an essential component of phytoene desaturation. Plant Cell 1995, 7:2139-2149] and a plastid terminal oxidase as electron acceptors [Carol P, Kuntz M: A plastid terminal oxidase comes to light: implications for carotenoid biosynthesis and chlororespiration. Trends Plant Sci 2001, 6:31-36]. When co-expressed in E. coli, PDS and ZDS from Arabidopsis convert phytoene to 7,9,7′,9′-tetra-cis-lycopene (poly-cis lycopene, ‘pro-lycopene’), while the bacterial CRTI phytoene desaturase produces all-trans lycopene [Bartley G E , Scolnik P A, Beyer P: Two arabidopsis thaliana carotene desaturases, phytoene desaturase and z-carotene desaturase, expressed in Escherichia coli, catalyze a poly-cis pathway to yield pro-lycopene. Eur.J.Biochem. 1999, 259:396-403]. The mechanism of carotenoid isomerization is yet unknown. However, it is predicted that a gene product of the locus tangerine (t) in tomato is involved in this process. In fruits of the recessive mutant tangerine lycopene is replaced by poly-cis lycopene and different isomers of up stream intermediates, such as neorosporene, zeta-carotene and phytofluene.

Cyclization of lycopene marks a branching point in the pathway; one route is leading to β-carotene and its derivative xanthophylls, and the other leading to α-carotene and lutein. Lycopene β-cyclase (LCY-B, CRTL-B) catalyzes a two-step reaction that creates one β-ionone ring at each end of the lycopene molecule to produce β-carotene, whereas lycopene ε-cyclase (LCY-E, CRTL-E) creates one ε-ring to give δ-carotene. It is presumed that α-carotene (β,ε-carotene) is synthesized by both enzymes. In view of the occurrence of a heterodimeric lycopene β-cyclase in Gram-positive bacteria [Krubasik P, Sandmann G: A carotenogenic gene cluster from Brevibacterium linens with novel lycopene cyclase genes involved in the synthesis of aromatic carotenoids. Mol.Gen.Genet. 2000, 263:423-432; Viveirosa M, Krubasikb P, Sandmannb G, Houssaini-Iraquic M: Structural and functional analysis of the gene cluster encoding carotenoid biosynthesis in Mycobacteriuni aurum A+. FEMS Microbiol.Lett. 2000, 187:95-101], it is alluring to consider that lycopene cyclases in plants work as dimmers as well. In this case it is possible that α-carotene is synthesized by a LCY-B/LCY-E heterodimer. Interestingly, lettuce (Lactuca sativa) contains a bi-cyclase CRTL-E that converts lycopene to ε-carotene [Cunningham F X, Jr., Gantt E: One ring or two? Determination of ring number in carotenoids by lycopene e-cyclases. Proc.Natl.Acad.Sci.U.S.A. 2001, 98:2905-2910]. There is a high degree of structural resemblance, 30% identity in amino acid sequence, between LCY-B and LCY-E in both tomato and Arabidopsis. The two enzymes contain a characteristic FAD/NAD(P)-binding sequence motif at the amino termini of the mature polypeptides. In tomato there are two lycopene β-cyclase enzymes, LCY-B (CRTL-B) [Pecker I, Gabbay R, Cunningham F X Jr, Hirschberg J: Cloning and characterization of the cDNA for lycopene beta-cyclase from tomato reveals decrease in its expression during fruit ripening. Plant Mol.Biol. 1996, 30:807-819] and CYC-B (‘B-cyclase’) [Ronen G, Carmel-Goren L, Zamir D, Hirschberg J: An alternative pathway to b-carotene formation in plant chromoplasts discovered by map-based cloning of Beta and old-gold color mutations in tomato. Proc.Natl.Acad.Sci.U.S.A. 2000, 97:11102-11107], whose amino acid sequences are 53% identical. LCY-B is active in green tissues, whereas CYC-B functions in chromoplast-containing tissues only. Interestingly, the amino acid sequence of CYC-B is more similar (86.1% identical) to capsanthin-capsorubin synthase (CCS) from pepper, an enzyme that converts antheraxanthin and violaxanthin to the red xanthophylls capsanthin and capsorubin, respectively [Bouvier F, Hugueney P, d'Harlingue A, Kuntz M, Camara B: Xanthophyll biosynthesis in chromoplasts: Isolation and molecular cloning of an enzyme catalyzing the conversion of 5,6-epoxycarotenoid into ketocarotenoid. Plant J. 1994, 6:45-54]. A deletion mutation in the Ccs gene (locus y), which results in the accumulation of violaxanthin, is responsible for the recessive phenotype of yellow fruit in pepper [Lefebvre V, Kuntz M, Camara B, Palloix A: The capsanthin-capsorubin synthase gene: a candidate gene for the y locus controlling the red fruit colour in pepper. Plant Mol.Biol. 1998, 36:785-789]. CCS exhibits low activity of lycopene β-cyclase when expressed in E. coli [Hugueney P, Badillo A, Chen H C, Klein A, Hirschberg J, Camara B, Kuntz M: Metabolism of cyclic carotenoids: A model for the alteration of this biosynthetic pathway in Capsicum annuum chromoplasts. Plant J. 1995, 8:417-424]. Similarities in function, gene structure and map position, strongly suggest that the genes Ccs and Cyc-b are orthologs that have originated by a gene duplication event from a common ancestor, most probably Lcy-b [Ronen G, Carmel-Goren L, Zamir D, Hirschberg J: An alternative pathway to b-carotene formation in plant chromoplasts discovered by map-based cloning of Beta and old-gold color mutations in tomato. Proc.Natl.Acad.Sci.U.S.A. 2000, 97:11102-11107]. While in tomato the duplicated gene has retained its original catalytic function, the second cyclase in pepper acquired during evolution a new enzymatic activity of a similar biochemical nature. Conservation of amino acid sequences as well as similar mechanisms of catalysis suggest that all plant cyclases, including CCS and perhaps also neoxanthin synthase, have evolved from a common ancestor, most probably the cyanobacterial CrtL.

Hydroxylation of cyclic carotenes at the 3C, 3′C positions is carried out by two types of enzymes, one is specific for β-rings and the other for ε-rings [Sun Z R, Gantt E, Cunningham F X Jr: Cloning and functional analysis of the β-carotene hydroxylase of Arabidopsis thaliana. J.Biol.Chem. 1996, 271:24349-24352; Pogson B, Mcdonald K A, Truong M, Britton G, Dellapenna D: Arabidopsis carotenoid mutants demonstrate that lutein is not essential for photosynthesis in higher plants. Plant Cell 1996, 8:1627-1639]. The β-carotene hydroxylase is ferredoxin dependent and requires iron, features characteristic of enzymes that exploit iron-activated oxygen to oxygenate carbohydrates [Bouvier F, Keller Y, d'Harlingue A, Camara B: Xanthophyll biosynthesis: molecular and functional characterization of carotenoid hydroxylases from pepper fruits (Capsicum annuum L.). Biochim.Biophys.Acta 1998, 1391:320-328]. Consequently, β-carotene is converted to zeaxanthin via β-cryptoxanthin. There are two β-carotene hydroxylases in both Arabidopsis [Kaul S, Analysis of the genome sequence of the flowering plant Arabidopsis thaliana. Nature 2000, 408:796-815] and tomato [GenBank Accession numbers: Y14810 and Y14809]. In the latter, one hydroxylase is expressed in green tissues and the other is exclusively expressed in the flower (unpublished data). The gene that ncodes for the ε-ring hydroxylase has not been identified yet. Zeaxanthin epoxidase (Zep1, ABA2) converts zeaxanthin to violaxanthin via antheraxanthin by introducing 5,6-epoxy groups into the 3-hydroxy-β-rings in a redox reaction that requires reduced ferredoxin [Bouvier F, d'Harlingue A, Hugueney P, Marin E, Marionpoll A, Camara B: Xanthophyll biosynthesis—Cloning, expression, functional reconstitution, and regulation of beta-cyclohexenyl carotenoid epoxidase from pepper (Capsicum annuum). J.Biol.Chem. 1996, 271:28861-28867]. Zep1 was cloned from Nicotiana plombaginifolia [Marin E, Nussaume L, Quesada A, Gonneau M, Sotta B, Hugueney P, Frey A, Marionpoll A: Molecular identification of zeaxanthin epoxidase of Nicotiana plumbaginifolia, a gene involved in abscisic acid biosynthesis and corresponding to the ABA locus of Arabidopsis thaliana. EMBO J. 1996, 15:2331-2342] and pepper [Bouvier F, d'Harlingue A, Hugueney P, Marin E, Marionpoll A, Camara B: Xanthophyll biosynthesis—Cloning, expression, functional reconstitution, and regulation of beta-cyclohexenyl carotenoid epoxidase from pepper (Capsicum annuum). J.Biol.Chem. 1996, 271:28861-28867]. In leaves violaxanthin can be converted back to zeaxanthin by violaxanthin deepoxidase (VDE), an enzyme that is activated by low pH generated in the chloroplast lumen under strong light. Zeaxanthin is effective in thermal dissipation of excess excitation energy in the light-harvesting antennae and thus plays a key role in protecting the photosynthetic system against damage by strong light. The inter-conversion of zeaxanthin and violaxanthin is known also as the “xanthophyll cycle”. Lack of the xanthophyll cycle in the Arabidopsis mutant npq1, due to a null mutation in Vde, increases the sensitivity of the plants to high light [Niyogi K K, Grossman A R, Bjorkman O: Arabidopsis mutants define a central role for the xanthophyll cycle in the regulation of photosynthetic energy conversion. Plant Cell 1998, 10:1121-1134]. The Vde gene was originally cloned from lettuce [Bugos R C, Yamamoto H Y: Molecular cloning of violaxanthin de-epoxidase from romaine lettuce and expression in Escherichia coli. Proc.Natl.Acad.Sci.U.S.A. 1996, 93:6320-6325]. The amino acid sequences of ZEP and VDE indicate that they are members of the lipocalins, a group of proteins that bind and transport small hydrophobic molecules [Hieber A D, Bugos R C, Yamamoto H Y: Plant lipocalins: violaxanthin de-epoxidase and zeaxanthin epoxidase. Biochim.Biophys.Acta 2000, 1482:84-91].

Carotenoid pigments are essential components in all photosynthetic organisms. They assist in harvesting light energy and protect the photosynthetic apparatus against harmful reactive oxygen species that are produced by over-excitation of chlorophyll. They also furnish distinctive yellow, orange and red colors to fruits and flowers to attract animals. Carotenoids are typically 40-carbon isoprenoids, which consist of eight isoprene units. The polyene chain in carotenoids contains up to 15 conjugated double bonds, a feature that is responsible for their characteristic absorption spectra and specific photochemical properties. These double bonds enable the formation of cis-trans geometric isomers in various positions along the molecule. Indeed, while the bulk of carotenoids in higher plants occur in the all-trans configuration, different cis isomers exist as well however in small proportions.

As discussed above, in plants, carotenoids are synthesized within the plastids from the central isoprenoid pathway [reviewed in Hirschberg 2001 Carotenoid biosynthesis in flowering plants Curr. Opin. Plant Biol. 4:210-218], which is incorporated herein by reference; summarized in FIG. 1). The first carotenoid in the committed pathway is phytoene, which is produced by the enzyme phytoene synthase (PSY) through a condensation of two molecules of geranylgeranyl diphosphate (GGDP). Four double bonds are subsequently introduced to phytoene by two enzymes, phytoene desaturase (PDS) and ζ-carotene desaturase (ZDS), each catalyzes two symmetric dehydrogenation steps to yield ζ-carotene and lycopene, respectively. It is recognized that cis-trans isomerizations do take place in vivo since phytoene is synthesized in the 15-cis configuration, while most of the further carotenoids are found in the all-trans form [Britton, G. (1988). Biosynthesis of carotenoids. In Plant Pigments, T. W. Goodwin, ed. (London and New York: Academic Press), pp. 133-180]. Furthermore, a small proportion of cis-isomers exist in many carotenoid species, for example 9-cis and 13-cis isomers of β-carotene, zeaxanthin and violaxanthin. However, the process of carotenoid isomerization has remained unexplained. The existence of a potential carotene isomerase enzyme could be expected from the phenotype of recessive mutation in tomato [Tomes, M. L., Quackenbush, F. W., Nelson, O. E., and North, B. (1953). The inheritance of carotenoid pigment system in the tomato. Genetics 38, 117-127] which accumulates prolycopene (7Z, 9Z, 7′Z, 9′Z tetra-cis lycopene), as well as poly-cis isomers of phytofluen, ζ-carotene and neurosporene [Zechmeister, L., LeRosen, A. L., Went, F. W., and Pauling, L. Prolycopene, a naturally occurring stereoisomer of lycopene. Proc. Natl. Acad. Sci. USA 1941: 27, 468-474; Clough, J. M., and Pattenden, G.: Naturally occurring poly-cis carotenoids: Stereochemistry of poly-cis lycopene and in congeners in ‘tangerine’ tomato fruits. J. Chem. Soc. Chem. Commun. 1979: 14, 616-619]. Co-expression of phytoene desaturase and ζ-carotene desaturase from Arabidopsis thaliana in Escherichia coli cells that synthesized phytoene produced mainly pro-lycopene whereas all-trans lycopene was produced in these cells by the bacterial phytoene desaturase [Bartley, G. E., Scolnik, P. A., and Beyer, P. (1999). Two Arabidopsis thaliana carotene desaturases, phytoene desaturase and ζ-carotene desaturase, expressed in Escherichia coli, catalyze a poly-cis pathway to yield pro-lycopene. Eur. J. Biochem. 259, 396-403]. This result supports the hypothesis that an active isomerization function is required in conjunction of the plant-type carotene desaturation reactions that yield lycopene, however, as of yet, no such enzymatic activity was described.

In view of the importance of carotenoids in physiological systems as well as the pigment and coloration industries, there is a widely recognized need for, and it would be highly advantageous to have, polypeptides having carotenoids isomerase catalytic activity and nucleic acids encoding same, which polypeptides and nucleic acids can be used in a variety of applications as is further delineated hereinbelow.

### SUMMARY OF THE INVENTION

While reducing the present invention to practice, map-based cloning was used to clone the gene that encodes the recessive mutation tangerine (t) [Tomes, M. L. (1952). Flower color modification associated with the gene t. Rep. Tomato Genet. Coop. 2, 12] in tomato (Lycopersicon esculentum). Fruits of tangerine are orange and accumulate prolycopene (7Z, 9Z, 7′Z, 9′Z tetra-cis lycopene) instead of the all-trans lycopene [(Zechmeister, L., LeRosen, A. L., Went, F. W., and Pauling, L. Prolycopene, a naturally occurring stereoisomer of lycopene. Proc. Natl. Acad. Sci. USA 1941: 27, 468-474; Clough, J. M., and Pattenden, G. Naturally occurring poly-cis carotenoids: Stereochemistry of poly-cis lycopene and in congeners in ‘tangerine’ tomato fruits. J. Chem. Soc. Chem. Commun. 1979: 14, 616-619)], which is normally synthesized in wild type fruits. The phenotype of tangerine is manifested also in yellowish young leaves and sometimes light green foliage and in pale colored flowers. The data presented herein indicates that the tangerine gene, designated CrtISO, encodes a redox-type enzyme that is structurally related to the bacterial-type phytoene desaturase, CRTI.

According to one aspect of the present invention there is provided an isolated nucleic acid comprising a polynucleotide encoding a polypeptide having an amino acid sequence at least 75, at least 80, at least 85, at least 90, at least 95 or at least 100%, similar (=identical acids+homologous acids) to SEQ ID NO:15, as determined using the Standard protein-protein BLAST [blastp] software of the NCBI, the polypeptide having carotenoids isomerase catalytic activity.

According to another aspect of the present invention there is provided an isolated nucleic acid comprising a polynucleotide at least 75, at least 80, at least 85, at least 90, at least 95 or at least 100% identical to positions 421-2265 of SEQ ID NO:14 or to positions 1341-6442 of SEQ ID NO:16, as determined using the Standard nucleotide-nucleotide BLAST [blastn] software of the NCBI.

According to further features in preferred embodiments of the invention described below, the polynucleotide comprises a cDNA.

According to still further features in the described preferred embodiments the polynucleotide comprises a genomic DNA.

According to still further features in the described preferred embodiments the polynucleotide comprises at least one intron sequence.

According to still further features in the described preferred embodiments the polynucleotide is intronless.

According to still further features in the described preferred embodiments the isolated nucleic acid further comprising a promoter operably linked to the polynucleotide in a sense orientation.

According to still further features in the described preferred embodiments the isolated nucleic acid further comprising a promoter operably linked to the polynucleotide in an antisense orientation.

According to yet another aspect of the present invention there is provided a vector comprising any of the isolated nucleic acids described herein.

According to further features in preferred embodiments of the invention described below, the vector is suitable for expression in a eukaryote.

According to still further features in the described preferred embodiments the vector is suitable for expression in a prokaryote.

According to still further features in the described preferred embodiments the vector is suitable for expression in a plant.

According to still another aspect of the present invention there is provided a transduced organism genetically transduced by any of the nucleic acids or vectors described herein, whereby the organism is a eukaryote, e.g., a plant, or prokaryote, e.g., a bacteria or cyanobacteria.

According to an additional aspect of the present invention there is provided a transduced cell expressing from a transgene a recombinant polypeptide having an amino acid sequence at least 50%, at least 55%, at least 60%, at least 65%, at least 70%, at least 75%, at least 80%, at least 85%, at least 90%, at least 95% or 100% similar to SEQ ID NO:15, as determined using the Standard protein-protein BLAST [blastp] software of the NCBI, the polypeptide having a carotenoids isomerase catalytic activity, the cell having a level of the carotenoids isomerase catalytic activity over that of a non-transduced and otherwise similar cell, whereby the cell is a eukaryote cell, e.g., a plant cell, or a prokaryote cell, e.g., a bacteria or cyanobacteria, wherein, the cell can be either isolated, grown in culture or form a part of an organism, e.g., a transgenic organism such as a transgenic plant.

According to yet an additional aspect of the present invention there is provided a transgenic plant having cells expressing from a transgene a recombinant polypeptide having an amino acid sequence at least 50%, at least 55%, at least 60%, at least 65%, at least 70%, at least 75%, at least 80%, at least 85%, at least 90%, at least 95% or 100% similar to SEQ ID NO:15, as determined using the Standard protein-protein BLAST [blastp] software of the NCBI, the polypeptide having a carotenoids isomerase catalytic activity, the cell having a level of the carotenoids isomerase catalytic activity over that of a non-transduced and otherwise similar cell.

According to still an additional aspect of the present invention there is provided a method of increasing a content of all-trans geometric isomers of carotenoids in a carotenoids producing cell, the method comprising, expressing in the cell, from a transgene, a recombinant polypeptide having an amino acid sequence at least 50%, at least 55%, at least 60%, at least 65%, at least 70%, at least 75%, at least 80%, at least 85%, at least 90%, at least 95% or 100% similar to SEQ ID NO:15, as determined using the Standard protein-protein BLAST [blastp] software of the NCBI, the polypeptide having a carotenoids isomerase catalytic activity.

According to a further aspect of the present invention there is provided a method of decreasing a content of all-trans geometric isomers of carotenoids in a carotenoids producing cell, the method comprising, expressing in the cell, from a transgene, a RNA molecule capable of reducing a level of a natural RNA encoding a carotenoids isomerase in the cell.

According to further features in preferred embodiments of the invention described below, the RNA molecule is antisense RNA, operative via antisense inhibition.

According to still further features in the described preferred embodiments the RNA molecule is sense RNA, operative via RNA inhibition.

According to still further features in the described preferred embodiments the RNA molecule is a ribozyme, operative via ribozyme cleavage inhibition.

According to still further features in the described preferred embodiments the RNA molecule comprises a sequence at least 50%, at least 55% at least 60%, at least 65%, at least 70%, at least 75%, at least 80%, at least 85%, at least 90%, at least 95% or 100% complementary (=identical to complementary strand) to a stretch of at least 15, at least 16, at least 17, at least 18, at least 19, at least 20, at least 25, at least 30, at least 40, at least 50, at least 60, at least 100, at least 200, at least 300, at least 500, at least 700, at least 1000 or at least 2000 contiguous nucleotides between positions 421-2265 of SEQ ID NO:14, as determined using the Standard nucleotide-nucleotide BLAST [blastn] software of the NCBI.

According to yet a further aspect of the present invention there is provided a method of modulating a ratio between all-trans geometric isomers of carotenoids and cis-carotenoids in a carotenoids producing cell, the method comprising, expressing in the cell, from a transgene, a RNA molecule capable of modulating a level of RNA encoding a carotenoids isomerase in the cell.

According to still further features in the described preferred embodiments the RNA molecule is sense RNA augmenting a level of the RNA encoding the carotenoids isomerase, thereby increasing the ratio.

According to still further features in the described preferred embodiments the RNA molecule comprises a sequence at least 50%, at least 55%, at least 60%, at least 65%, at least 70%, at least 75%, at least 80%, at least 85%, at least 90%, at least 95% or 100% identical to positions 421-2265 of SEQ ID NO:14, as determined using the Standard nucleotide-nucleotide BLAST [ blastn] software of the NCBI, and encoding a polypeptide having a carotenoids isomerase catalytic activity.

According to further features in preferred embodiments of the invention described below, the RNA molecule is antisense RNA, operative via antisense inhibition, thereby decreasing the ratio.

According to still further features in the described preferred embodiments the RNA molecule is sense RNA, operative via RNA inhibition, thereby decreasing the ratio.

According to still further features in the described preferred embodiments the RNA molecule is a ribozyme, operative via ribozyme cleavage inhibition, thereby decreasing the ratio.

According to still further features in the described preferred embodiments the RNA molecule comprises a sequence at least 50%, at least 55%, at least 60%, at least 65%, at least 70%, at least 75%, at least 80%, at least 85%, at least 90%, at least 95% or 100% complementary to a stretch of at least 15, at least 16, at least 17, at least 18, at least 19, at least 20, at least 25, at least 30, at least 40, at least 50, at least 60, at least 100, at least 200, at least 300, at least 500, at least 700, at least 1000 or at least 2000 contiguous nucleotides between positions 421-2265 of SEQ ID NO:14, as determined using the Standard nucleotide-nucleotide BLAST [blastn] software of the NCBI.

According to still a further aspect of the present invention there is provided a method of decreasing a content of all-trans geometric isomers of carotenoids in a carotenoids producing cell, the method comprising, introducing into the cell an antisense nucleic acid molecule capable of reducing a level of a natural mRNA encoding a carotenoids isomerase in the cell via at least one antisense mechanism.

According to further features in preferred embodiments of the invention described below, the antisense nucleic acid molecule is antisense RNA.

According to still further features in the described preferred embodiments the antisense nucleic acid molecule is an antisense oligonucleotide of at least 15, at least 16, at least 17, at least 18, at least 19, at least 20, at least 25, at least 30, at least 40, at least 50, at least 60, or at least 100 nucleotides.

According to still further features in the described preferred embodiments the antisense nucleic acid molecule comprises a sequence at least 50%, at least 55%, at least 60%, at least 65%, at least 70%, at least 75%, at least 80%, at least 85%, at least 90%, at least 95% or 100% complementary to a stretch of at least 15, at least 16. at least 17. at least 18, at least 19, at least 20, at least 25, at least 30, at least 40, at least 50, at least 60, at least 100, at least 200, at least 300, at least 500, at least 700, at least 1000 or at least 2000 contiguous nucleotides between positions 421-2265 of SEQ ID NO:14, as determined using the Standard nucleotide-nucleotide BLAST [blastn] software of the NCBI.

According to still further features in the described preferred embodiments the oligonucleotide is a synthetic oligonucleotide and comprises a man-made modification rendering the synthetic oligonucleotide more stable in cell environment.

According to still further features in the described preferred embodiments the synthetic oligonucleotide is selected from the group consisting of methylphosphonate oligonucleotide, monothiophosphate oligonucleotide, dithiophosphate oligonucleotide, phosphoramidate oligonucleotide, phosphate ester oligonucleotide, bridged phosphorothioate oligonucleotide, bridged phosphoramidate oligonucleotide, bridged methylenephosphonate oligonucleotide, dephospho internucleotide analogs with siloxane bridges, carbonate bridge oligonucleotide, carboxymethyl ester bridge oligonucleotide, carbonate bridge oligonucleotide, carboxymethyl ester bridge oligonucleotide, acetamide bridge oligonucleotide, carbamate bridge oligonucleotide, thioether bridge oligonucleotide, sulfoxy bridge oligonucleotide, sulfono bridge oligonucleotide and α-anomeric bridge oligonucleotide.

According to another aspect of the present invention there is provided an expression construct for directing an expression of a gene-of-interest in a plant tissue, the expression construct comprising a regulatory sequence of CrtISO of tomato.

According to further features in preferred embodiments of the invention described below, the plant tissue is selected from the group consisting of flower, fruit and leaves.

According to still another aspect of the present invention there is provided a method of isolating a polynucleotide encoding a polypeptide having an amino acid sequence at least 50%, at least 55%, at least 60%, at least 65%, at least 70%, at least 75%, at least 80%, at least 85%, at least 90%, at least 95% or 100% similar to SEQ ID NO:15 and hence potentially having a carotenoids isomerase catalytic activity from a carotenoid producing species, the method comprising screening a cDNA or genomic DNA library prepared from isolated RNA or genomic DNA extracted from the species with a nucleic acid probe of at least 15, at least 16, at least 17, at least 18, at least 19, at least 20, at least 25, at least 30, at least 40, at least 50, at least 60, at least 100, at least 200, at least 300, at least 500, at least 700, at least 1000 or at least 2000 nucleotides and being at least 50% identical to a contiguous stretch of nucleotides of SEQ ID NO:14 or 16 or their complementary sequences and isolating clones reacting with the probe.

According to yet another aspect of the present invention there is provided a method of isolating a polynucleotide encoding a polypeptide having an amino acid sequence at least 50% similar to SEQ ID NO:15 and hence potentially having a carotenoids isomerase catalytic activity from a carotenoid producing species, the method comprising providing at least one PCR primer of at least 15, at least 16, at least 17, at least 18, at least 19, at least 20, at least 25, at least 30, at least 40, at least 50, at least 60 or at least 100 nucleotides being at least 50%, at least 55%, at least 60%, at least 65%, at least 70%, at least 75%, at least 80%, at least 85%, at least 90%, at least 95% or 100% identical to a contiguous stretch of nucleotides of SEQ ID NO:14 or 16 or their complementary sequences and using the at least one PCR primer in a PCR reaction to amplify at least a segment of the polynucleotide from DNA or cDNA derived from the species.

According to still another aspect of the present invention there is provided an isolated polypeptide comprising an amino acid sequence at least 75%, at least 80%, at least 85%, at least 90%, at least 95% or 100% similar to SEQ ID NO:15, as determined using the Standard protein-protein BLAST [blastp] software of the NCBI, the polypeptide having carotenoids isomerase catalytic activity.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a scheme presenting the carotenoid biosynthesis pathway in plants; and

FIG. 2 is a scheme demonstrating the organization of the genomic sequences of the CrtISO gene from tomato (Lycopersicon esculentum). Filled boxes represent exons. Deletions found in CrtISO of tangerine alleles are indicated. Bar under the map corresponds to 1 kb.

FIG. 3 demonstrates the expression of CrtISO during tomato fruit development. Steady-state levels of mRNA of CrtISO, Psy and Pds were measured by RT-PCR from total RNA isolated from different stages of fruit development wild-type (WT) L. esculentum (M82) and mutant tangerine 3183. PCR products were separated by agarose gel electrophoresis and stained with ethidium bromide. G, mature green fruit; B, breaker stage; R, ripe stage 7 days after breaker. 1/3×B and 3×B are samples which contained three times or one third the total RNA from breaker stage fruits.

FIGS. 4A-B are schemes demonstrating the targeted insertion mutagenesis of gene s110033 in Synechocystis PCC 6803. FIG. 4A is a scheme demonstrating the homologous recombination event between the cloned s110033 and the chromosomal gene. FIG. 4B is a scheme demonstrating the resulting insertion with the spectinomycin resistance gene.

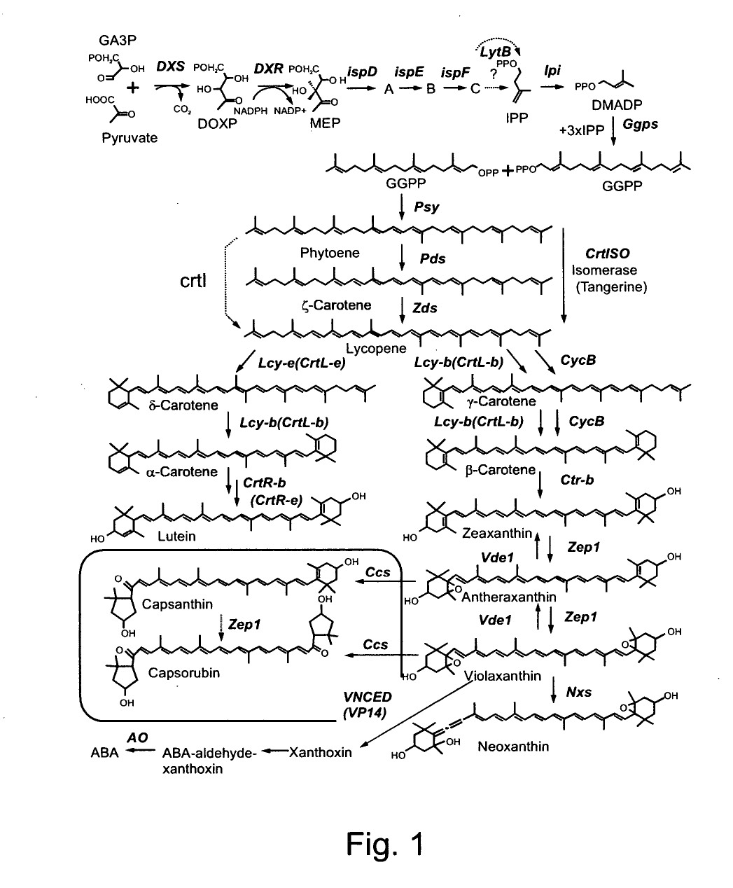

### DESCRIPTION OF THE PREFERRED EMBODIMENTS

The present invention is of (i) polypeptides having carotenoids isomerase catalytic activity; (ii) preparations including same; (iii) nucleic acids encoding same; (iv) nucleic acids controlling the expression of same; (v) vectors harboring the nucleic acids; (vi) cells and organisms, inclusive plants, algae, cyanobacteria and naturally non-photosynthetic cells and organisms, genetically modified to express the carotenoids isomerase; and (vii) cells and organisms, inclusive plants, algae and cyanobacteria that naturally express a carotenoids isomerase and are genetically modified to reduce its level of expression.

The principles and operation of the various aspects of the present invention may be better understood with reference to the drawings, examples and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

While reducing the present invention to practice, map-based cloning was used to clone the gene that encodes the recessive mutation tangerine (t) [Tomes, M. L. (1952). Flower color modification associated with the gene t. Rep. Tomato Genet. Coop. 2, 12] in tomato (Lycopersicon esculentum). Fruits of tangerine are orange and accumulate prolycopene (7Z, 9Z, 7′Z, 9′Z tetra-cis lycopene) instead of the all-trans lycopene, which is normally synthesized in wild type fruits [Zechmeister, L., LeRosen, A. L., Went, F. W., and Pauling, L. Prolycopene, a naturally occurring stereoisomer of lycopene. Proc. Nat. Acad. Sci. USA 1941: 27, 468-474; Clough, J. M., and Pattenden, G. Naturally occurring poly-cis carotenoids: Stereochemistry of poly-cis lycopene and in congeners in ‘tangerine’ tomato fruits. J. Chem. Soc. Chem. Commun. 1979: 14, 616-619]. The phenotype of tangerine is manifested also in yellowish young leaves and sometimes light green foliage and in pale colored flowers. The data presented herein indicates that the tangerine gene, designated CrtISO, encodes a redox-type enzyme that is structurally related to the bacterial-type phytoene desaturase, CRTI.

According to one aspect of the present invention there is provided an isolated nucleic acid comprising a polynucleotide encoding a polypeptide having an amino acid sequence at least 75, at least 80, at least 85, at least 90, at least 95 or at least 100%, similar to SEQ ID NO:15, as determined using the Standard protein-protein BLAST [blastp] software of the NCBI, the polypeptide having carotenoids isomerase catalytic activity.

As used herein, the terms “nucleic acid” and “polynucleotide” refer to any polymeric sequence of nucleobases capable of base-pairing with a complementary DNA or RNA. Hence a polynucleotide or a nucleic acid may be natural or synthetic and may include natural or analog nucleobases.

As used herein the term “similar” refers to the sum of identical amino acids and homologous amino acids, as accepted in the art.

As used herein, the phrase “carotenoids isomerase catalytic activity” refers to an enzymatic activity which reduces the activation energy for the conversion of a cis double bond in a carotenoid to a trans double bond, whereby conversion of all cis double bonds in a carotenoid results in an all-trans carotenoid.

As used herein the term “cis-carotenoid” refers to a carotenoid having at least one double-bond connecting two carbons in a cis orientation.

According to another aspect of the present invention there is provided an isolated nucleic acid comprising a polynucleotide at least 75, at least 80, at least 85, at least 90, at least 95 or at least 100% identical to positions 421-2265 of SEQ ID NO:14 (positions 421-2265 of SEQ ID NO:14 constitute the open reading frame of the CrtISO gene of tomato) or to positions 1341-6442 of SEQ ID NO:16 (positions 1341-6442 of SEQ ID NO:16 constitute the exons and introns of the CrtISO gene of tomato), as determined using the Standard nucleotide-nucleotide BLAST [blastn] software of the NCBI.

The polynucleotide of the present invention can be, for example, a cDNA or a genomic DNA isolated from a carotenoids producing organism or it can be a composite DNA, including mixed cDNA and genomic DNA sequences, derived from one or more carotenoids producing organisms, combined into an operative gene which may include one or more introns and one or more exons, or no introns at all (i.e., intronless), to direct the transcription of a mRNA that, when properly spliced, encodes any of the polypeptides of the present invention.

Alternatively or additionally, the polynucleotide according to this aspect of the present invention is hybridizable with SEQ ID NOs: 14, 16, 19 and/or 21.

Hybridization for long nucleic acids (e.g., above 200 bp in length) is effected preferably under stringent or moderate hybridization, wherein stringent hybridization is effected by a hybridization solution containing 10% dextrane sulfate, 1 M NaCl, 1% SDS and 5×10⁶ cpm ³²p labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1% SDS and 5×10⁶ cpm ³²p labeled probe, at 65° C., with a final wash solution of 1×SSC and 0.1% SDS and final wash at 50° C.

Isolating novel DNA sequences having potential carotenoids isomerase catalytic activity can be done either by conventional screening of DNA or cDNA libraries or by PCR amplification of DNA or cDNA, using probes or PCR primers derived from the CrtISO gene of tomato. Such probes and such PCR primers both form a part of the present invention. The preparation and use of such probes and PCR primers are well known in the art. Further details pertaining to the preparation and use of such probes and PCR primers can be found in numerous text books, including, for example, in “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); “Oligonucleotide Synthesis” Gait, M. J., ed. (1984); “Nucleic Acid Hybridization” Hames, B. D., and Higgins S. J., eds. (1985); “A Practical Guide to Molecular Cloning” Perbal, B., (1984); and “PCR Protocols: A Guide To Methods And Applications”, Academic Press, San Diego, Calif. (1990).

Hence, according to still another aspect of the present invention there is provided a method of isolating a polynucleotide encoding a polypeptide having an amino acid sequence at least 50%, at least 55%, at least 60%, at least 65%, at least 70%, at least 75%, at least 80%, at least 85%, at least 90%, at least 95% or 100% similar to SEQ ID NO:15 and hence potentially having a carotenoids isomerase catalytic activity from a carotenoid producing species, the method comprising screening a cDNA or genomic DNA library prepared from isolated RNA or genomic DNA extracted from the species with a nucleic acid probe of at least 15, at least 16, at least 17, at least 18, at least 19, at least 20, at least 25, at least 30, at least 40, at least 50, at least 60, at least 100, at least 200, at least 300, at least 500, at least 700, at least 1000 or at least 2000 nucleotides and being at least 50% identical to a contiguous stretch of nucleotides of SEQ ID NO:14 or 16 or their complementary sequences and isolating clones reacting with the probe.

According to yet another aspect of the present invention there is provided a method of isolating a polynucleotide encoding a polypeptide having an amino acid sequence at least 50% similar to SEQ ID NO:15 and hence potentially having a carotenoids isomerase catalytic activity from a carotenoid producing species, the method comprising providing at least one PCR primer of at least 15, at least 16, at least 17, at least 18, at least 19, at least 20, at least 25, at least 30, at least 40, at least 50, at least 60 or at least 100 nucleotides being at least 50%, at least 55%, at least 60%, at least 65%, at least 70%, at least 75%, at least 80%, at least 85%, at least 90%, at least 95% or 100% identical to a contiguous stretch of nucleotides of SEQ ID NO:14 or 16 or their complementary sequences and using the at least one PCR primer in a PCR reaction to amplify at least a segment of the polynucleotide from DNA or cDNA derived from the species.

The nucleic acids of the present invention may include a promoter operably linked to the polynucleotide in a sense or antisense orientation.

As used herein, the term “sense” is used to describe a sequence which has a % identity or is identical to a reference sequence.

As used herein, the term “antisense” is used to describe a sequence which has a % identity or is identical to a sequence which is complementary to a reference sequence.

As such, the phrase “sense orientation” refers to an orientation which will result in the transcription of a sense RNA, whereas the phrase “antisense orientation” refers to an orientation which will result in the transcription of an antisense RNA.

According to another aspect of the present invention there is provided a vector comprising any of the isolated nucleic acids described herein. The vector of the present invention is suitable for expression in a eukaryote, such as a higher plant, or in a prokaryote, such as a bacteria or a cyanobacteria. The vector of the present invention, as well as optional constituents thereof and methods of using same in stable and/or transient transformation and/or transfection protocols are further described in detail hereinafter.

According to still another aspect of the present invention there is provided a transduced organism genetically transduced by any of the nucleic acids or vectors described herein, whereby the organism can be a eukaryote, e.g., a plant, or a prokaryote, e.g., a bacteria or a cyanobacteria. Methods of stably and/or transiently transducing via transformation and/or transfection a variety of eukaryote and/or prokaryote organisms are further described in detail hereinafter.

Hence, according to an additional aspect of the present invention there is provided a transduced cell expressing from a transgene a recombinant polypeptide having an amino acid sequence at least 50%, at least 55%, at least 60%, at least 65%, at least 70%, at least 75%, at least 80%, at least 85%, at least 90%, at least 95% or 100% similar to SEQ ID NO:15, as determined using the Standard protein-protein BLAST [blastp] software of the NCBI, the polypeptide having a carotenoids isomerase catalytic activity, the cell having a level of the carotenoids isomerase catalytic activity over that of a non-transduced and otherwise similar cell, whereby the cell is a eukaryote cell, e.g., a plant cell, or a prokaryote cell, e.g., a bacteria or cyanobacteria, wherein, the cell can be either isolated, grown in culture or form a part of an organism, e.g., a transgenic organism such as a transgenic plant.

Similarly, according to yet an additional aspect of the present invention there is provided a transgenic plant having cells expressing from a transgene a recombinant polypeptide having an amino acid sequence at least 50%, at least 55%, at least 60%, at least 65%, at least 70%, at least 75%, at least 80%, at least 85%, at least 90%, at least 95% or 100% similar to SEQ ID NO:15, as determined using the Standard protein-protein BLAST [blastp] software of the NCBI, the polypeptide having a carotenoids isomerase catalytic activity, the cell having a level of the carotenoids isomerase catalytic activity over that of a non-transduced and otherwise similar cell.

According to still an additional aspect of the present invention there is provided a method of increasing a content of all-trans geometric isomers of carotenoids in a carotenoids producing cell, the method comprising, expressing in the cell, from a transgene, a recombinant polypeptide having an amino acid sequence at least 50%, at least 55%, at least 60%, at least 65%, at least 70%, at least 75%, at least 80%, at least 85%, at least 90%, at least 95% or 100% similar to SEQ ID NO:15, as determined using the Standard protein-protein BLAST [blastp] software of the NCBI, the polypeptide having a carotenoids isomerase catalytic activity.

Thus, this aspect of the present invention provides polynucleotides, which encode polypeptides exhibiting carotenoids isomerase catalytic activity. The isolated polynucleotides of the present invention can be expressed in variety of single cell or multicell expression systems.

According to another aspect of the present invention there is provided an isolated polypeptide comprising an amino acid sequence at least 75%, at least 80%, at least 85%, at least 90%, at least 95% or 100% similar to SEQ ID NO:15, as determined using the Standard protein-protein BLAST [blastp] software of the NCBI, the polypeptide having carotenoids isomerase catalytic activity. The polypeptide of the present invention can be expressed using the polynucleotides and vectors of the present invention in a variety of expression systems, for a variety of applications, ranging from interfering in carotenoids biosynthesis in vivo to the isolation of the polypeptide, all as is further delineated hereinbelow in detail.

For expression in a single cell system, the polynucleotides of the present invention are cloned into an appropriate expression vector (i.e., construct).

Depending on the host/vector system utilized, any of a number of suitable transcription and translation elements including constitutive and inducible promoters, transcription enhancer elements, transcription terminators, and the like, can be used in the expression vector [see, e.g., Bitter et al., (1987) Methods in Enzymol. 153:516-544].

Other then containing the necessary elements for the transcription and translation of the inserted coding sequence, the expression construct of the present invention can also include sequences engineered to enhance stability, production, purification and yield of the expressed polypeptide. For example, the expression of a fusion protein or a cleavable fusion protein comprising a polypeptide of the present invention and a heterologous protein can be engineered. Such a fusion protein can be designed so as to be readily isolated by affinity chromatography; e.g., by immobilization on a column specific for the heterologous protein. Where a cleavage site is engineered between the protein of interest and the heterologous protein, the protein of interest can be released from the chromatographic column by treatment with an appropriate enzyme or agent that disrupts the cleavage site [ e.g., see Booth et al. ( 1988) Immunol. Lett. 19:65-70; and Gardella et al., (1990) J. Biol. Chem. 265:15854-15859].

In one embodiment, the polypeptide encoded by the nucleic acid molecule of the present invention includes an N terminal transit peptide fused thereto which serves for directing the polypeptide to a specific membrane. Such a membrane can be, for example, the cell membrane or such a membrane can be the outer and preferably the inner chloroplast membrane. Transit peptides which function as herein described are well known in the art. Further description of such transit peptides is found in, for example, Johnson et al. The Plant Cell (1990) 2:525-532; Sauer et al. EMBO J. (1990) 9:3045-3050; Mueckler et al. Science (1985) 229:941-945; Von Heijne, Eur. J. Biochem. (1983) 133:17-21; Yon Heijne, J. Mol. Biol. (1986) 189:239-242; Iturriaga et al. The Plant Cell (1989) 1:381-390; McKnight et al., Nucl. Acid Res. (1990) 18:4939-4943; Matsuoka and Nakamura, Proc. Natl. Acad. Sci. USA (1991) 88:834-838. A recent text book entitled “Recombinant proteins from plants”, Eds. C. Cunningham and A. J. R. Porter, 1998 Humana Press Totowa, N.J. describe methods for the production of recombinant proteins in plants and methods for targeting the proteins to different compartments in the plant cell. The book by Cunningham and Porter is incorporated herein by reference. It will however be appreciated by one of skills in the art that a large number of membrane integrated proteins fail to poses a removable transit peptide. It is accepted that in such cases a certain amino acid sequence in said proteins serves not only as a structural portion of the protein, but also as a transit peptide.

A variety of cells can be used as host-expression systems to express the isomerase coding sequence. These include, but are not limited to, microorganisms, such as bacteria transformed with a recombinant bacteriophage DNA, plasmid DNA or cosmid DNA expression vector containing the isomerase coding sequence; yeast transformed with recombinant yeast expression vectors containing the isomerase coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the isomerase coding sequence (further described in the specifications hereinunder). Mammalian expression systems can also be used to express the isomerases. Bacterial systems are preferably used to produce recombinant isomerase, according to the present invention, thereby enabling a high production volume at low cost.

In bacterial systems, a number of expression vectors can be advantageously selected depending upon the use intended for isomerase expressed. For example, when large quantities of isomerase are desired, vectors that direct the expression of high levels of protein product, possibly as a fusion with a hydrophobic signal sequence, which directs the expressed product into the periplasm of the bacteria or the culture medium where the protein product is readily purified may be desired. Certain fusion protein engineered with a specific cleavage site to aid in recovery of the isomerase may also be desirable. Such vectors adaptable to such manipulation include, but are not limited to, the pET series of E. coli expression vectors [Studier et al. (1990) Methods in Enzymol. 185:60-89).

It will be appreciated that when codon usage for isomerase cloned from plants is inappropriate for expression in E. coli, the host cells can be co-transformed with vectors that encode species of tRNA that are rare in E. coli but are frequently used by plants. For example, co-transfection of the gene dnaY, encoding tRNA_{ArgAGA/AGG}, a rare species of tRNA in E. coli, can lead to high-level expression of heterologous genes in E. coli. [Brinkmann et al., Gene 85:109 (1989) and Kane, Curr. Opin. Biotechnol. 6:494 (1995)]. The dnaY gene can also be incorporated in the expression construct such as for example in the case of the pUBS vector (U.S. Pat. No. 6,270,0988).

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

Other expression systems such as insects and mammalian host cell systems, which are well known in the art can also be used by the present invention.

Transformed cells are cultured under conditions, which allow for the expression of high amounts of recombinant isomerase. Such conditions include, but are not limited to, media, bioreactor, temperature, pH and oxygen conditions that permit protein production. Media refers to any medium in which a cell is cultured to produce the recombinant isomerase protein of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Depending on the vector and host system used for production, resultant proteins of the present invention may either remain within the recombinant cell; be secreted into the fermentation medium; be secreted into a space between two cellular membranes, such as the periplasmic space in E. coli; or be retained on the outer surface of a cell or viral membrane.

Recovery of the recombinant protein is effected following an appropriate time in culture. The phrase “recovering the recombinant protein refers to collecting the whole fermentation medium containing the protein and need not imply additional steps of separation or purification. Not withstanding from the above, proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatography focusing and differential solubilization.

Polypeptide expression in plants, is effected by transforming plants with the polynucleotide sequences of the present invention.

For effecting plant transformation, the polynucleotides which encode isomerases are preferably included within a nucleic acid construct or constructs which serve to facilitates the introduction of the exogenous polynucleotides into plant cells or tissues and to express these enzymes in the plant.

The nucleic acid constructs according to the present invention are utilized to express in either a transient or preferably a stable manner the isomerase encoding polynucleotide of the present invention within a whole plant, defined plant tissues, or defined plant cells.

Thus, according to a preferred embodiment of the present invention, the nucleic acid constructs further include a promoter for regulating the expression of the isomerase encoding polynucleotide of the present invention.

Numerous plant functional expression promoters and enhancers which can be either tissue specific, developmentally specific, constitutive or inducible can be utilized by the constructs of the present invention, some examples are provided hereinunder.

As used herein in the specification and in the claims section that follows the phrase “plant promoter” or “promoter” includes a promoter which can direct gene expression in plant cells (including DNA containing organelles). Such a promoter can be derived from a plant, bacterial, viral, fungal or animal origin. Such a promoter can be constitutive, i.e., capable of directing high level of gene expression in a plurality of plant tissues, tissue specific, i.e., capable of directing gene expression in a particular plant tissue or tissues, inducible, i.e., capable of directing gene expression under a stimulus, or chimeric, i.e., formed of portions of at least two different promoters.

Thus, the plant promoter employed can be a constitutive promoter, a tissue specific promoter, an inducible promoter or a chimeric promoter.

Examples of constitutive plant promoters include, without being limited to, CaMV35S and CaMV19S promoters, FMV34S promoter, sugarcane bacilliform badnavirus promoter, CsVMV promoter, Arabidopsis ACT2/ACT8 actin promoter, Arabidopsis ubiquitin UBQ1 promoter, barley leaf thionin BTH6 promoter, and rice actin promoter.

Examples of tissue specific promoters include, without being limited to, bean phaseolin storage protein promoter, DLEC promoter, PHSβ promoter, zein storage protein promoter, conglutin gamma promoter from soybean, AT2S1 gene promoter, ACT11 actin promoter from Arabidopsis, napA promoter from Brassica napus and potato patatin gene promoter.

The inducible promoter is a promoter induced by a specific stimuli such as stress conditions comprising, for example, light, temperature, chemicals, drought, high salinity, osmotic shock, oxidant conditions or in case of pathogenicity and include, without being limited to, the light-inducible promoter derived from the pea rbcS gene, the promoter from the alfalfa rbcS gene, the promoters DRE, MYC and MYB active in drought; the promoters INT, INPS, prxEa, Ha hsp17.7G4 and RD21 active in high salinity and osmotic stress, and the promoters hsr203J and str246C active in pathogenic stress.

The construct according to the present invention preferably further includes an appropriate and unique selectable marker, such as, for example, an antibiotic resistance gene. In a more preferred embodiment according to the present invention the constructs further include an origin of replication.

The constructs according to the present invention can be a shuttle vector, which can propagate both in E. coli (wherein the construct comprises an appropriate selectable marker and origin of replication) and be compatible for propagation in cells, or integration in the genome, of a plant.

There are various methods of introducing nucleic acid constructs into both monocotyledonous and dicotyledenous plants (Potrykus, I., Annu. Rev. Plant. Physiol., Plant. Mol. Biol. (1991) 42:205-225; Shimamoto et al., Nature (1989) 338:274-276). Such methods rely on either stable integration of the nucleic acid construct or a portion thereof into the genome of the plant, or on transient expression of the nucleic acid construct in which case these sequences are not inherited by a progeny of the plant.

There are two principle methods of effecting stable genomic integration of exogenous sequences such as those included within the nucleic acid constructs of the present invention into plant genomes:
(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.
(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; or by the direct incubation of DNA with germinating pollen, DeWet et al. in Experimental Manipulation of Ovule Tissue, eds. Chapman, G. P. and Mantell, S. H. and Daniels, W. Longman, London, (1985)p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988)p. 1-9. A supplementary approach employs the Agrobacterium delivery system in combination with vacuum infiltration. The Agrobacterium system is especially viable in the creation of transgenic dicotyledenous plants.

There are various methods of direct DNA transfer into plant cells. In electroporation, protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals, tungsten particles or gold particles, and the microprojectiles are physically accelerated into cells or plant tissues.

Following transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Transient expression methods which can be utilized for transiently expressing the isolated nucleic acid included within the nucleic acid construct of the present invention include, but are not limited to, microinjection and bombardment as described above but under conditions which favor transient expression, and viral mediated expression wherein a packaged or unpackaged recombinant virus vector including the nucleic acid construct is utilized to infect plant tissues or cells such that a propagating recombinant virus established therein expresses the non-viral nucleic acid sequence.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, the constructions can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of the present invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that these sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of the present invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at-least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

It will be appreciated that co-transformation of the polynucleotides of the present invention together with other polynucleotides is desirable to achieve a synergistic effect, such as the combination of isomerases and other genes participating in carotenoids synthesis.

Any plant species may be transformed with the nucleic acid constructs of the present invention including species of gymnosperms as well as angiosperms, dicotyledonous plants as well as monocotyledonous plants which are commonly used in agriculture, horticulture, forestry, gardening, indoor gardening, or any other form of activity involving plants, either for direct use as food or feed, or for further processing in any kind of industry, for extraction of substances, for decorative purposes, propagation, cross-breeding or any other use.

Generally, after transformation plant cells or explants are selected for the presence of one or more markers, which are encoded by the constructed vector of the present invention, whereafter the transformed material is regenerated/propagated into a whole plant.

The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transgenic plant be produced such that the regenerated plant has the identical traits and characteristics of the parent transgenic plant. Therefore, it is preferred that the transgenic plant be regenerated by micropropagation which provides a rapid, consistent reproduction of the transgenic plants.

Micropropagation is a process of growing new generation plants from a single piece of tissue that has been excised from a selected parent plant or cultivar. This process permits the mass reproduction of plants having the preferred tissue expressing the fusion protein. The new generation plants, which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transgenic plantlets are transferred to a greenhouse for hardening where the plants' tolerance to light is gradually increased so that it can be grown in the natural environment.

Following plant transformation and propagation, selection of appropriate plants can be effected by monitoring the expression levels of the exogenous isomerase or by monitoring the transcription levels of the corresponding mRNA.

The expression levels of the exogenous isomerase can be determined using immunodetection assays (i.e., ELISA and western blot analysis, immunohistochemistry and the like), which may be effected using antibodies specifically recognizing the recombinant polypeptide. Methods of antibody generation are disclosed in “Cellular and Molecular immunology” Abbas, K. et al. (1994) 2nd ed. W B Saunders Comp ed. which is fully incorporated herein. Alternatively, the recombinant polypeptides can be monitored by SDS-PAGE analysis using different staining techniques, such as but not limited to, coomassie blue or silver staining.

Messenger RNA (mRNA) levels of the polypeptides of the present invention may also be indicative of the transformation rate and/or level. mRNA levels can be determined by a variety of methods known to those of skill in the art, such as by hybridization to a specific oligonucleotide probe (e.g., Northern analysis) or RT-PCR.

To specifically detect the polynucleotide sequences of the present invention, measures are taken to design specific oligonucleotide probes, which would not hybridize with other related genes under the hybridization conditions used.

Hybridization of short nucleic acids (below 200 bp in length, e.g. 17-40 bp in length) can be effected by the following hybridization protocols depending on the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ; (ii) hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 37° C., final wash solution of 6×SSC and final wash at 22° C.

The oligonucleotides of the present invention can be used in any technique which is based on nucleotide hybridization including, subtractive hybridization, differential plaque hybridization, affinity chromatography, electrospray mass spectrometry, northern analysis, RT-PCR and the like. For PCR-based methods a pair of oligonucleotides is used in an opposite orientation so as to direct exponential amplification of a portion thereof in a nucleic acid amplification reaction, such as a polymerase chain reaction. The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., ideally between 3° C. and 0° C.

Whenever required, any of the above transformation/transfection techniques may be employed to practice the following aspects and preferred embodiments of the present invention.

The isolated sequences prepared as described herein, can be used to prepare expression cassettes useful in a number of techniques. For example, expression cassettes of the invention can be used to suppress endogenous isomerase gene expression. Inhibiting expression can be useful, for instance, in suppressing the production of all-trans carotenoids in some or all plant parts, so as to achieve coloration effects.

A number of methods can be used to inhibit gene expression in plants. For instance, antisense technology can be conveniently used. To accomplish this, a nucleic acid segment from the desired gene is cloned and operably linked to a promoter such that the antisense strand of RNA will be transcribed. The expression cassette is then transformed into plants and the antisense strand of RNA is produced. In plant cells, it has been suggested that antisense RNA inhibits gene expression by preventing the accumulation of mRNA which encodes the enzyme of interest, see, e.g., Sheehy, et al., Proc. Nat. Acad. Sci. USA, 85:8805-8809 (1988), and Hiatt et al., U.S. Pat. No. 4,801,340.

The nucleic acid segment to be introduced generally will be substantially identical to at least a portion of the endogenous gene or genes to be repressed.

The sequence, however, need not be perfectly identical to inhibit expression. The vectors of the present invention can be designed such that the inhibitory effect applies to other proteins within a family of genes exhibiting homology or substantial homology to the target gene.

For antisense suppression, the introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. Normally, a sequence of between about 30 or 40 nucleotides and about full length nucleotides should be used, though a sequence of at least about 100 nucleotides is preferred, a sequence of at least about 200 nucleotides is more preferred, and a sequence of at least about 500 nucleotides is especially preferred.

Catalytic RNA molecules or ribozymes can also be used to inhibit expression of carotenoids isomerase genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs.

A number of classes of ribozymes have been identified. One class of ribozymes is derived from a number of small circular RNAs which are capable of self-cleavage and replication in plants. The RNAs replicate either alone (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, solanum nodiflorum mottle virus and subterranean clover mottle virus. The design and use of target RNA-specific ribozymes is described in Haseloff, et al., Nature 334:585-591 (1988).

As is further detailed hereinunder antisense oligonucleotides can also be used for suppression of gene expression.

Another method of suppression is sense suppression, also known as RNA inhibition (RNAi). Introduction of expression cassettes in which a nucleic acid is configured in the sense orientation with respect to the promoter has been shown to be an effective means by which to block the transcription of target genes. For an example of the use of this method to modulate expression of endogenous genes see, Napoli, et al., The Plant Cell 2:279-289 (1990), and U.S. Pat. Nos. 5,034,323, 5,231,020, and 5,283,184.

Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity might exert a more effective repression of expression of the endogenous sequences. Substantially greater identity of more than about 80% is preferred, though about 95% to absolute identity would be most preferred. As with antisense regulation, the effect should apply to any other proteins within a similar family of genes exhibiting homology or substantial homology.

For sense suppression, the introduced sequence in the expression cassette, needing less than absolute identity, also need not be full length, relative to either the primary transcription product or fully processed mRNA. This may be preferred to avoid concurrent production of some plants which are overexpressers. A higher identity in a shorter than full length sequence compensates for a longer, less identical sequence. Furthermore, the introduced sequence need not have the same intron or exon pattern, and identity of non-coding segments will be equally effective. Normally, a sequence of the size ranges noted above for antisense regulation is used.

Hence, according to a further aspect of the present invention there is provided a method of decreasing a content of all-trans geometric isomers of carotenoids in a carotenoids producing cell, the method comprising, expressing in the cell, from a transgene, a RNA molecule capable of reducing a level of a natural RNA encoding a carotenoids isomerase in the cell. The RNA molecule can be antisense RNA, operative via antisense inhibition, sense RNA, operative via RNA inhibition or a ribozyme, operative via ribozyme cleavage inhibition.

The RNA molecule preferably comprises a sequence at least 50%, at least 55%, at least 60%, at least 65%, at least 70%, at least 75%, at least 80%, at least 85%, at least 90%, at least 95% or 100% complementary to a stretch of at least 15, at least 16, at least 17, at least 18, at least 19, at least 20, at least 25, at least 30, at least 40, at least 50, at least 60, at least 100, at least 200, at least 300, at least 500, at least 700, at least 1000 or at least 2000 contiguous nucleotides between positions 421-2265 of SEQ ID NO:14, as determined using the Standard nucleotide-nucleotide BLAST [blastn] software of the NCBI.

As used herein, the phrase “% complementary” means % identity to a complementary sequence of a sequence identified by it's SEQ ID NO.

According to yet a further aspect of the present invention there is provided a method of modulating a ratio between all-trans geometric isomers of carotenoids and cis-carotenoids in a carotenoids producing cell, the method comprising, expressing in the cell, from a transgene, a RNA molecule capable of modulating a level of RNA encoding a carotenoids isomerase in the cell.

According to one embodiment, the RNA molecule is sense RNA augmenting a level of the RNA encoding the carotenoids isomerase, thereby increasing the ratio. For example, the RNA molecule comprises a sequence at least 50%, at least 55%, at least 60%, at least 65%, at least 70%, at least 75%, at least 80%, at least 85%, at least 90%, at least 95% or 100% identical to positions 421-2265 of SEQ ID NO:14, as determined using the Standard nucleotide-nucleotide BLAST [blastn] software of the NCBI, and encoding a polypeptide having a carotenoids isomerase catalytic activity.

According to another embodiment, the RNA molecule is antisense RNA, operative via antisense inhibition, thereby decreasing the ratio.

According to yet another embodiment the RNA molecule is sense RNA, operative via RNA inhibition, thereby decreasing the ratio.

According to still another embodiment, the RNA molecule is a ribozyme, operative via ribozyme cleavage inhibition, thereby decreasing the ratio.

For example, the RNA molecule comprises a sequence at least 50%, at least 55%, at least 60%, at least 65%, at least 70%, at least 75%, at least 80%, at least 85%, at least 90%, at least 95% or 100% complementary to a stretch of at least 15, at least 16, at least 17, at least 18, at least 19, at least 20, at least 25, at least 30, at least 40, at least 50, at least 60, at least 100, at least 200, at least 300, at least 500, at least 700, at least 1000 or at least 2000 contiguous nucleotides between positions 421-2265 of SEQ ID NO:14, as determined using the Standard nucleotide-nucleotide BLAST [blastn] software of the NCBI.

According to still a further aspect of the present invention there is provided a method of decreasing a content of all-trans geometric isomers of carotenoids in a carotenoids producing cell, the method comprising, introducing into the cell an antisense nucleic acid molecule capable of reducing a level of a natural mRNA encoding a carotenoids isomerase in the cell via at least one antisense mechanism.

According to one embodiment, the antisense nucleic acid molecule is antisense RNA or the antisense nucleic acid molecule is an antisense oligonucleotide of at least 15, at least 16, at least 17, at least 18, at least 19, at least 20, at least 25, at least 30, at least 40, at least 50, at least 60, or at least 100 nucleotides.

The antisense nucleic acid molecule preferably comprises a sequence at least 50%, at least 55%, at least 60%, at least 65%, at least 70%, at least 75%, at least 80%, at least 85%, at least 90%, at least 95% or 100% complementary to a stretch of at least 15, at least 16, at least 17, at least 18, at least 19, at least 20, at least 25, at least 30, at least 40, at least 50, at least 60, at least 100, at least 200, at least 300, at least 500, at least 700, at least 1000 or at least 2000 contiguous nucleotides between positions 421-2265 of SEQ ID NO:14, as determined using the Standard nucleotide-nucleotide BLAST [blastn] software of the NCBI.

The oligonucleotide is preferably a synthetic oligonucleotide and comprises a man-made modification rendering the synthetic oligonucleotide more stable in cell environment. Examples include, without limitation, methylphosphonate oligonucleotide, monothiophosphate oligonucleotide, dithiophosphate oligonucleotide, phosphoramidate oligonucleotide, phosphate ester oligonucleotide, bridged phosphorothioate oligonucleotide, bridged phosphoramidate oligonucleotide, bridged methylenephosphonate oligonucleotide, dephospho internucleotide analogs with siloxane bridges, carbonate bridge oligonucleotide, carboxymethyl ester bridge oligonucleotide, carbonate bridge oligonucleotide, carboxymethyl ester bridge oligonucleotide, acetamide bridge oligonucleotide, carbamate bridge oligonucleotide,thioether bridge oligonucleotide, sulfoxy bridge oligonucleotide, sulfono bridge oligonucleotide and α-anomeric bridge oligonucleotide.

Antisense oligonucleotides for use in can be designed following the teachings of Biotechnol Bioeng, 1999, 5;65(1):1-9 “Prediction of antisense oligonucleotide binding affinity to a structured RNA target” by Walton S P, Stephanopoulos G N, Yarmush M L, Roth C M; and “Prediction of antisense oligonucleotide efficacy by in vitro methods” by O. Matveeva, B. Felden, A. Tsodikov, J. Johnston, B. P. Monia, J. F. Atkins, R. F. Gesteland & S. M. Freier Nature Biotechnology 16, 1374-1375 (1998).

According to another aspect of the present invention there is provided an expression construct for directing an expression of a gene-of-interest in a plant tissue, the expression construct comprising a regulatory sequence of CrtISO of tomato. This promoter is useful in directing gene expression in, for example, flowers, fruits and leaves.

The expression construct according to the present invention may include, in addition to the regulatory sequence of CrtISO of tomato, any of the elements described above with respect to plasmid and viral expression constructs (vectors) and may hence serve in any of the transformation/transfection protocols described herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorpotaed by reference as if fully set forth herein. Other general references are provided throughout this document. The procedures therein are believed to be well known in the art and are provided for the convenience of the reader. All the information contained therein is incorporated herein by reference.

### Materials and Experimental Methods

Plant Material and Growth Conditions

Lycopersicon esculentum CV M-82 and the introgression line IL 10-2 [Eshed, Y. and Zamir, D. (1995). An introgression line population of Lycopersicon pennellii in the cultivated tomato enables the identification and fine mapping of yield-associated QTL. Genetics 141, 1147-1162] served as the wild-type tomato lines. The tangerine mutant LA3183 (tangerine³¹⁸³), which was kindly provided by Roger Chetelat, the Tomato Genetics Resource Center, University of California, Davis, was used for mapping the locus t and for characterization of the phenotype. Mutant tangerine^{mic} was identified among M2 plants of fast neutron mutagenesis of Micro-Tom tomato [Meissner, R., Jacobson, Y., Melamed, S., Levyatuv, S., Shalev, G., Ashri, A., Elkind, Y., and Levy, A. A. (1997). A new model system for tomato genetics. Plant J. 12, 1465-1472] and was kindly donated by Avi Levy, The Weizmann Institute, Rehovot, Israel.

Recombinants in the F2 generation of a cross between tangerine³¹⁸³ and IL10-2 were selfed and the F3 progeny were screened for homozygous recombination products. Fixed recombinant plants were used for fine mapping the locus t and served as isogenic lines for carotenoid analysis and measurement of gene expression. Lines 98-802 and 98-818 served as wild type and lines 98-823 and 104 served as tangerine³¹⁸³.

Seeds of the different lines were sterilized by soaking in 70% ethanol for 2 minutes, in 3.3% NaOCl and 0.1% TWEEN 20 for 10 minutes, followed by three washes with sterile water. Seeds were sowed on Murshige and Skoog (MS) basal salt mixture with 3% sucrose. The seedlings were grown in 23° C. in dark or light for two weeks before leaves were analyzed. Plants were grown in the field for crossing and in the greenhouse for fruit analysis.

Carotenoid Analysis

Extraction of carotenoids from tomato fruits followed previously described protocols [Ronen, G., Cohen, M., Zamir, D., and Hirschberg, J. (1999). Regulation of carotenoid biosynthesis during tomato fruit development: expression of the gene for lycopene epsilon-cyclase is down-regulated during ripening and is elevated in the mutant delta. Plant J. 17, 341-351; Ronen, G., Carmel-Goren, L., Zamir, D., and Hirschberg, J. (2000). An alternative pathway to β-carotene formation in plant chromoplasts discovered by map-based cloning of Beta and old-gold color mutations in tomato. Proc. Natl. Acad. Sci. U.S.A. 97, 11102-11107]. Leaf pigments were extracted from ˜70 mg of fresh cotyledons of dark or light grown seedlings. Fresh tissue was minced in acetone and filtered. The solvent was dried under stream of nitrogen and dissolved in acetone. Flower pigments were extracted from petals of fresh single flowers (for Micro-Tom two flowers were extracted for each sample). The tissues were ground in 2 ml of acetone; then 2 ml of dichloromethane were added and the samples were agitated until all pigments were extracted. Saponification of flower carotenoids was done in ethanol/KOH (60% w/vol), 9:1 for 16 hours at 4° C., The carotenoids were extracted with ether after addition of NaCl to a final concentration of 1.2%. The samples were dried and dissolved in acetone. Analysis by HPLC using photo-diode array detector has been previously described [Ronen, G., Cohen, M., Zamir, D., and Hirschberg, J. (1999). Regulation of carotenoid biosynthesis during tomato fruit development: expression of the gene for lycopene epsilon-cyclase is down-regulated during ripening and is elevated in the mutant delta. Plant J. 17, 341-351; Ronen, G., Carmel-Goren, L., Zamir, D., and Hirschberg, J. (2000). An alternative pathway to β-carotene formation in plant chromoplasts discovered by map-based cloning of Beta and old-gold color mutations in tomato. Proc. Natl. Acad. Sci. U.S.A. 97, 11102-11107]. Carotenoids were identified by their characteristic absorption spectra, distinctive retention time and, in some cases, comparison of standards. Quantification was done by integrating the peak areas of the HPLC chromatogram using the MILLENIUM chromatography software (Waters).

Map-based Cloning Techniques

Genomic DNA was prepared from 5 g of leaf tissue as described [Eshed, Y. and Zamir, D. (1995). An introgression line population of Lycopersicon pennellii in the cultivated tomato enables the identification and fine mapping of yield-associated QTL. Genetics 141, 1147-1162]. Restriction fragment length polymorphism (RFLP) in genomic DNA from tomato was carried out with markers TG-408, CT-20, CD72, CT-57, TG-1 and TG-241 [Tanksley, S. D., Ganal, M. W., Prince, J. C., de Vicente, M. C., Bonierabale, M. W., Broun, P., Fulton, T. M., Giovanonni, J. J., Grandillo, S., Martin, G. B., Messeguer, R., Miller, J. C., Miller, L., Paterson, A. H., Pineda, O., Roder, M. S., Wing, R. A., Wu, W., and Young, N. D. (1992). High density molecular linkage maps of the tomato and potato genomes. Genetics 132, 1141-1160]. Genomic library in bacterial artificial chromosomes (BAC) of L. esculentum var Heinz1706 (http:\\www.clemson.edu) was screened with the marker DNA CT-57. Sequences at the ends of the insert in BAC₂₁O12 were amplified by PCR using the primers: BAC2FA, 5′-TGTCATCACCCAATTTTCCA-3′ (SEQ ID NO:1) (“for” end of BAC2); BAC2FB, 5′-TTCCAGGAACTTGGTTTCCTT-3′ (SEQ ID NO:2) (“for” end of BAC2); BAC2RA, 5′-TGAAAGGGCATACCAAAAGG-3′ (SEQ ID NO:3) (“rev” end of BAC2); BAC2RB 5′-GGCTACGCCAAGAACTCTGA-3′ (SEQ ID NO:4) (“rev” end of BAC2). The amplified sequences were used as probes in hybridization with DNA from recombinant plants. DNA fragments of the BAC insert were subcloned in the plasmid vector pBS (Promega) and sequenced using the T3 and T7 universal primers. Assembly of sequences was accomplished with the VECTOR NTI Suit software package. cDNA clones were obtained by reverse transcription (RT) followed by PCR using total RNA isolated from flowers.

Functional Expression in E. coli of Biosynthetic Enzymes

Plasmid pAC-Zeta, which carries the genes crtE and crtE from Erwinia and crtP from Synechococcus PCC7942, has been previously described [Cunningham, F. X. Jr., Sun, Z. R., Chamovitz, D., Hirschberg, J., and Gantt, E. (1994). Molecular structure and enzymatic function of lycopene cyclase from the cyanobacterium Synechococcus sp strain PCC7942. Plant Cell 6, 1107-1121]. Plasmid pGB-Ipi was constructed by inserting the cDNA of Ipi from Haematococcus pluvialis [Cunningham, F. X., Jr. and Gantt, E. (2001). One ring or two? Determination of ring number in carotenoids by lycopene ε-cyclases. Proc. Natl. Acad. Sci. U.S.A. 98, 2905-2910] (kindly provided by F. X. Cunningham, University of Maryland) into the HindII site of plasmid vector pGB2 [Churchward, G., Belin, D., and Nagamine, Y. (1984). A pSC101-derived plasmid which shows no sequence homology to other commonly used cloning vectors. Gene 31, 165-171]. Plasmid pCrtISO was constructed by subcloning a 1631 bp PCR amplified fragment from the cDNA of the tomato (L. esculentum cv M82) CrtISO. The primers used for amplification were: 5′GTTCTAGATGTAGACAAAAGAGTGGA3′ (SEQ ID NO:5) (forward) and 5′ ACATCTAGATATCATGCTAGTGTCCTT 3′ (SEQ ID NO:6) (reverse). Both primers contain a single mismatch to create an XbaI restriction site. The PCR fragment was cut with XbaI and subcloned into the XbaI site of vector pBluescriptSK⁻. Plasmid pT-Zds was constructed by subcloning a 1643 bp PCR amplified sequence from the tomato cDNA of Zds (GeneBank Accession No. AF195507). This DNA fragment was obtained using the primers Tzds248, 5′GCTGATTTGGATATCTATGGTTTC 3′ (SEQ ID NO:7) (forward) and TZds1901, 5′AACTCGAGTTGTATTTGGATGATTTGCA 3′ (SEQ ID NO:8) (reverse). The primers contain each a single mismatch to create EcoRV and Xho restriction sites, respectively. The PCR fragment was cut with EcoRV and XhoI and subcloned into a vector pBluescriptSK⁻, which was cut with SmaI and XhoI. Plasmid pCrtISO-TZds was constructed by subcloning the CrtISO cDNA fragment, which was excised from pCrtISO with the restriction endonucleases Cfr42I and BcuI, into pTZds, which was cut with the same enzymes.

E. coli cells of the strain XLI-Blue carrying plasmid pGB-Ipi were co-transformed with plasmids pAC-Zeta and pTzds, pCrtISO and pTzds-CrtISO in various combinations and selected on LB medium containing the appropriate antibiotics: spectinomycin (50 mg/l), ampicillin (100 mg/l) and chloramphenicol (50 mg/l).

Measurement of mRNA by RT-PCR

Protocols for RNA extraction and reverse transcription have been previously described [Ronen, G., Cohen, M., Zamir, D., and Hirschberg, J. (1999). Regulation of carotenoid biosynthesis during tomato fruit development: expression of the gene for lycopene epsilon-cyclase is down-regulated during ripening and is elevated in the mutant delta. Plant J. 17, 341-351; Ronen, G., Carmel-Goren, L., Zamir, D., and Hirschberg, J. (2000). An alternative pathway to β-carotene formation in plant chromoplasts discovered by map-based cloning of Beta and old-gold color mutations in tomato. Proc. Natl. Acad. Sci. U.S.A. 97, 11102-11107]. Total RNA was isolated from 1 gram of fruit tissue using the TRI-reagent® protocol (Molecular Research Center, Cincinnati). Following reverse transcription of total mRNA the cDNAs of Psy, Pds and CrtISO, were amplified by PCR that consisted of 24, 26 and 28 cycles, respectively, of 1 min at 95° C., 1 min at 56° C., and 1 min at 72° C. Various initial concentrations of mRNA, ranging over 9-fold difference, were used to demonstrate linear ratio between the concentration of template mRNA and the final PCR products. The following primers were used for PCR amplification:

Pds, 5′-TTGTGTTTGCCGCTCCAGTGGATAT-3′ (SEQ ID NO:9) (forward) and 5′-GCGCCTTCCATTGAAGCCAAGTAT-3′ (SEQ ID NO:10) (reverse); for Psy, 5′-GGGGAATTTGGGCTTGTTGAGT-3′ (SEQ ID NO:11) (forward) and 5′-CCTTTGATTCAGGGGCGATACC-3′ (SEQ ID NO:12) (reverse); for CrtISO, 5′-GATCGCCAAATCCTTAGCAA-3′ (SEQ ID NO:13) (forward) and 5′-GCCCTGGGAAGAGTGTTTTT-3′ (SEQ ID NO:24) (reverse). Products of the PCR amplification were separated by electrophoresis in 1.5% agarose gels and stained with ethidium bromide.

Transfection into Cyanobacteria

The following protocol was used to introduce DNA into the cyanobacteria Synechocystis PCC 6803. In general, cyanobacteria were grown in BG-11 medium [Rippka, R., Deruelles, J., Waterbury, J. B. Herdman, M. and Stanier, R. Y. (1979) “Generic assignment, strain histories and properties of pure culture of cyanobacteria.” Gen. Microbiol. 111:1-16] supplemented with 10 mM TES, pH 8.23 and 5 mM glucose. When needed, 20 μg/ml spectinomycin was added. The cyanobacteria were grown at 33° C. under continuous light of 30 μE. Small suspension cultures were grown in Erlenmeyer flasks on a rotary shaker. Large suspension cultures were grown in 1 liter flat bottles aerated with filtered air. When the bacteria were incubated in darkness the bottles were completely covered with aluminum foil. Plate medium was supplemented with 1.5% w/v Difco Bactoagar and 3% w/v sodium thiosulfate. The plates where kept at a relative humidity of approximately 80%. A fresh culture of cyanobacteria Synechocystis PCC 6803 was grown in BG-11 medium to a cell density of OD₇₂₀=0.6. 30 ml of the culture were centrifuged at 3000 g for 10 minutes and the supernatant was discarded. The cells pellet was resuspended in 30 ml of sterile 10 mM NaCl and the cells were centrifuged again under the same conditions and the supernatant was discarded. The cells were resuspended in fresh BG-11 medium to a concentration equivalent to OD₇₂₀=4.8. The cells suspension was divided into 400 μl aliquots and 5-10 μg DNA was added to an aliquot. Thereafter, the cultures were grown over night at 30° C. under continuous shaking conditions. The cultures were further grown for additional 24 hours in 50 ml fresh BG-11. The cultures were centrifuged at 2000 g for 10 minutes and the cells pellet was resuspended in 1 ml fresh BG-11. 100 μl aliquots were plated onto solid BG-11 petri plates supplemented with the appropriate antibiotics. Colonies appeared following seven days of incubation.

Since cyanobacteria contain multiple copies of the genome per cell, and assuming that initial incorporation of the introduced DNA into the genome occurs in only one gene copy, it is important to continue growing the transformants under selection of the appropriate antibiotics to enable complete segregation of the transformed genome. It usually requires four weeks of continuous propagation under selective conditions to obtain a pure mutant in Synechocystis PCC 6803 [Williams, J. G. K. (1988). “Construction of specific mutations in photosystem II photosynthetic reaction center by genetic engineering methods in Synechocystis PCC 6803.” Methods Enzymol. 167: 766-778].

DNA and Protein Sequence Analysis

Sequence of DNA was determined by the ABI Prism 377 DNA (Perkin Elmer) sequencer and processed with the ABI sequence analysis software. Vector NTI suit software (InforMax Inc., Bethesda, Md.) was used for sequence analysis.

### Experimental Results

Carotenoid Composition in Wild Type and Tangerine

Carotenoids accumulated in fruits and flowers of wild type and tangerine mutants were extracted and analyzed by HPLC (Tables I and II). In wild type, 75% of total carotenoids in ripe fruits (Table I) 7 days after breaker stage consisted of all-trans lycopene and less than 15% are lycopene precursors (neurosporene, ζ-carotene, phytofluene and phytoene). In fruits of mutant tangerine³¹⁸³ the major carotenoid accumulated is pro-lycopene whereas lycopene precursors, mostly in cis configuration, comprise most of the rest of the carotenoids. Only a small fraction of less than 2% is all-trans lycopene. In the tangerine^{mic} the phenotype is similar but more severe with the main carotenoids being cis-ζ-carotene (32%) and prolycopene (15%).

TABLE I
Carotenoid composition in tomato fruits

| | M-82 (WT) | tangerine³¹⁸³ | Micro-Tom | tangerine^{mic} |
|---|---|---|---|---|
| Phytoene | 5.2 ± 2.1 | 15.3 ± 2.6 | 6.9 ± 2.1 | 16.0 ± 1.2 |
| Phytofluene | 3.6 ± 0.8 | 8.7 ± 1.53 | 5.9 ± 1.0 | 9.8 ± 1.8 |
| ζ-carotene | 1.5 ± 2.0 | 23.6 ± 6.3 | 1.1 ± 0.4 | 31.7 ± 8.4 |
| Cis- neurosporene | | 8.0 ± 4.1 | | 11.4 ± 0.6 |
| Neurosporene | 0.2 ± 0.3 | 6.0 ± 3.1 | 0.4 ± 0.4 | 6.2 ± 1.7 |
| Di-Cis-lycopene | 0 | 6.4 ± 3.2 | 0 | 3.6 ± 0.3 |
| Prolycopene | 0.4 ± 0.7 | 25.4 ± 7.7 | 0 | 15.2 ± 7.8 |
| Lycopene | 75.2 ± 10.2 | 0.6 ± 1.2 | 78.0 ± 6.0 | 0 |
| β-carotene | 5.9 ± 3.1 | 2.8 ± 3.3 | 1.2 ± 0.4 | 2.4 ± 1.0 |
| Others (+unidentified) | 8.0 | 3.2 | 6.5 | 307 |
| Total carotenoids | 77.0 ± 5.0 | 66.0 ± 17.0 | 104.0 ± 33.0 | 53.0 ± 8.0 |
| (μg · g⁻¹ fresh tissue) | | | | |

Carotenoid composition in fruits of wild type and tangerine mutants. Unless otherwise indicated, numbers correspond to percent of total carotenoids.

In the flowers of the wild type the yellow xanthophylls, neoxanthin, violaxanthin and lutein, encompass 95% of total carotenoids (Table II). In contrast, the fraction of xanthophyls is less than 40 percent of total carotenoids in flowers of tangerine³¹⁸³ and less than 10 percent in tangerine^{mic}. Instead, prolycopene and its precursors accumulate in both cases.

TABLE II
Carotenoid composition in tomato flowers (μg · g⁻¹ flower tissue)

| | M-82 (WT) | tangerine³¹⁸³ | Micro-Tom | Tangerine^{mic} |
|---|---|---|---|---|
| Phytoene^{a} | 0 | 11.5 ± 3.4 | 0.3 ± 0.1 | 25.0 ± 7.8 |
| Phytofluene^{a} | 0 | 3.3 ± 0.9 | 0 | 7.5 ± 2.8 |
| ζ-carotene^{a} | 0 | 4.6 ± 1.9 | 0 | 8.8 ± 1.3 |
| Cis-neurosporene | | | | 2.6 ± 0.1 |
| Neurosporene | 0 | 1.6 ± 1.1 | 0 | 2.0 ± 0.1 |
| Di-cis-lycopene | 0 | 0 | 0 | 4.0 ± 3.8 |
| Prolycopene | 0 | 1.0 ± 0.9 | 0 | 30.2 ± 2.8 |
| Lycopene | 0 | 1.8 ± 1.8 | 0 | 0 |
| γ-carotene | 0 | 3.2 ± 1.6 | 0 | 0 |
| β-carotene | 1.1 ± 1.7 | 5.5 ± 1.4 | 0.8 ± 0.7 | 3.5 ± 1.6 |
| Rubixanthin | 0 | 11.6 ± 4.5 | 0 | 1.8 ± 2.6 |
| β-cryptoxanthin | 0 | 2.5 ± 0.7 | 0.9 ± 1.1 | 0 |
| Violaxanthin | 37.0 ± 7.1 | 11.0 ± 6.1 | 33.2 ± 6.4 | 0 |
| Neoxanthin | 59.4 ± 7.0 | 36.5 ± 9.7 | 57.7 ± 6.3 | 9.7 ± 6.5 |
| Lutein | 2.5 ± 1.0 | 4.8 ± 0.6 | 7.1 ± 2.3 | 0 |
| Others (+unidentified) | 0 | 1.5 | 0 | 4.9 |
| Total carotenoids | 770 ± 112 | 490 ± 327 | 1,350 ± 521 | 998 |
| (μg · g⁻¹ fresh tissue) | | | | |

^{a}All isomers.
Carotenoid composition in flowers of wild type and tangerine mutants. Unless otherwise indicated, numbers correspond to percent of total carotenoids.

The tangerine mutation affects carotenoid biosynthesis also in chloroplats as is evident by the yellow color that appears in the newly developed leaves. Leaves of etiolated seedlings of tangerine^{mic}, but not tangerine³¹⁸³ or wild type, accumulate pro-lycopene and its precursors and do not contain any xanthophylls (Table III). These data indicate that the locus tangerine is involved in carotenoid isomerization that is essential for biosynthesis of cyclized carotenes and xanthophylls.

TABLE III
Carotenoid composition (percent) in 7 days
old tomato seedlings of wild type (WT) and mutant
tangerin^{mic} grown in light or dark

| | |
|---|---|
| Light | Dark |

| | WT | Tangerin^{mic} | WT | Tangerin^{mic} |
|---|---|---|---|---|
| Phytoene | 0.6 | 0.7 | 0 | 17.8 |
| Phytofluene* | 0 | 0 | 0 | 7.7 |
| Zeta-carotene* | 0 | 0 | 0 | 24.2 |
| Neurosporene* | 0 | 0 | 0 | 15.1 |
| Prolycopene | 0 | 0 | 0 | 34.8 |
| Lycopene | 0 | 0 | 0 | 0 |
| Beta-carotene | 28 | 33 | 4 | 0 |
| Violaxanthin | 3.3 | 22.5 | 19.8 | 0 |
| Neoxanthin | 7.9 | 10.6 | 0 | 0 |
| Lutein | 59.6 | 29.2 | 76.2 | 0 |
| Others | 0.6 | 0.4 | 0 | 0.4 |

*All isomers

Map-based Cloning of the t Gene

The recessive mutation tangerine was mapped to the long arm of chromosome 10, 4 cM away from the locus l2. This locus is located in a region that overlaps with IL10-2. Because none of the known carotenoid biosynthesis genes maps near this locus (data not shown) it has been predicted that tangerine is determined by a new gene. To further map tangerinec, tt×IL10-2 were crossed and analyzed 1045 F2 plants using the markers TG408 and TG241 that flank tangerine. 218 recombinant plants were obtained and these individuals were selfed to determine their genotype with respect to the recessive mutation t. The recombinants were probed with additional markers and CT57 was found to co-segregate with tangerine. Genomic library of tomato in bacterial artificial chromosomes (BAC) [Budiman, M. A., Mao, L., Wood, T. C., and Wing, R. A. (2000). A deep-coverage tomato BAC library and prospects toward development of an STC framework for genome sequencing. Genome Res. 10, 129-136] was screened with CT57 and BAC 21O21 was identified. Sequences at the ends of the insert of BAC 21O21 were amplified by PCR and used as probes in genomic DNA hybridization of the 218 recombinant plants. The results indicated that BAC 21O21 contained the entire region of the tangerine locus because both BAC ends revealed recombinations with the target gene.

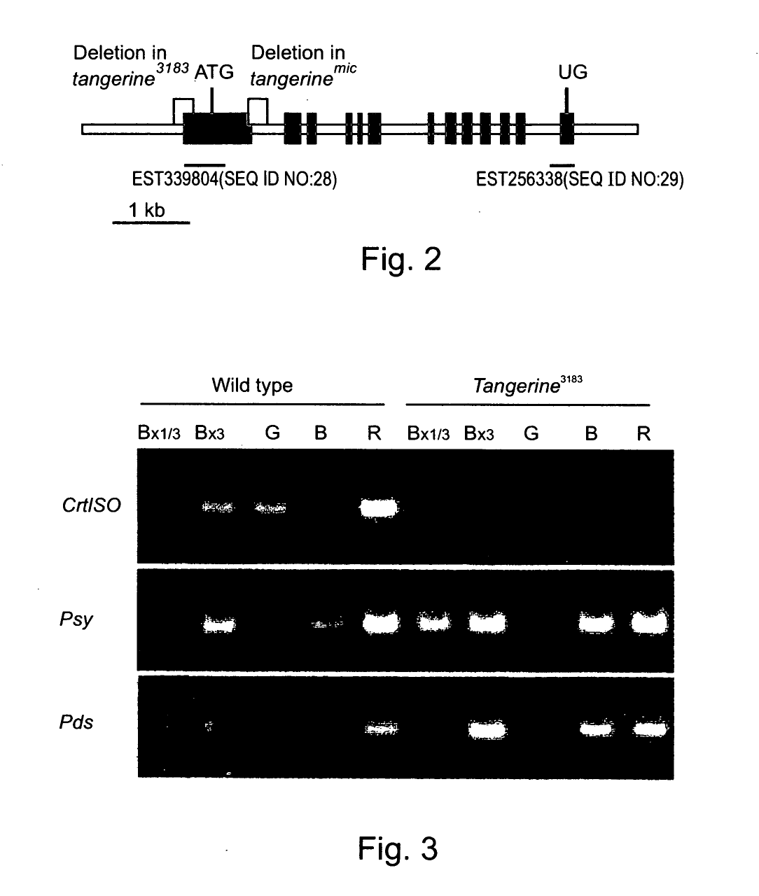

The entire insert of BAC 21O21 was sequenced. An open reading frame (ORF) sequence with some similarity to the bacterial gene for phytoene desaturase was found to co-segregate with the tangerine phenotype. The cDNA clone of this gene, CrtISO, was obtained by RT-PCR using primers 5′-TCTTGGGTTTCCAGCAATTT-3′ (forward primer) (SEQ ID NO:27) and 5′-GGAGGAACCTCAATTGGAACC-3′ (reverse primer) (SEQ ID NO:21) that were designed according to data from the tomato EST data bank [EST339804 (Accession No. AW738377, SEQ ID NO:28) and EST256338 (Accession No. A1775238, SEQ ID NO:29), see FIG. 2 for alignment of these EST sequences with the genomic sequence of CrtISO]. Comparison between the genomic and cDNA sequences revealed that the gene is composed of 13 exons and 12 introns. DNA blot hybridization with total genomic DNA indicated that CrtISO exits in a single copy in the tomato genome.

Sequence Analysis of CrtISO in Wild Type and Tangerine Alleles

The cDNA of CrtISO contains an ORF of 615 codons, which encodes a polypeptide of calculated molecular mass of 67.5 kDa. No differences in amino acid sequence were found between CRTISO from the wild type of cultivars M82, Ailsa Craig and Micro-Tom and the polypeptide in tangerine³¹⁸³. In contrast, analysis of both cDNA and genomic sequences of CrtISO from tangerine^{mic} indicated that this allele contained a deletion of 282 bp that encompasses 24 bp of the first exon and 258 bp of the first intron. Due to this deletion a splicing site is eliminated and the abnormal mRNA that is produced contains an early stop codon that aborts the synthesis of functional CRTISO. A delition of 348 bp was discovered in the promoter region of CRTISO of tangerine³¹⁸³.

The following Table IV summarizes the SEQ ID Nos. of the sequences described herein:

TABLE IV

| Sequence | SEQ ID NO: |
|---|---|
| CrtISO cDNA sequence, WT (ORF 421-2265) | 14 |
| CRTISO amino acids sequence, WT | 15 |
| CrtISO genomic DNA sequence, WT | 16 |
| CrtISO genomicDNA sequence, gene t³¹⁸³ | 17 |
| CrtISO genomic DNA sequence, gene t^{mic} | 18 |

The following Table V in conjunction with FIG. 2 describes the structure of the CrtISO, with respect to SEQ ID No:16:

TABLE V

| Identifier | Position(s) in SEQ ID NO: 16 |
|---|---|
| Promoter sequence: | 1-1341 |
| Transcription initiation: | 1341 |
| Exon No. 1: | 1341-2236 |
| Exon No. 2: | 2665-2871 |
| Exon No. 3: | 2962-3061 |
| Exon No. 4: | 3453-3535 |
| Exon No. 5: | 3623-3679 |
| Exon No. 6: | 3760-3915 |
| Exon No. 7: | 4548-4623 |
| Exon No. 8: | 4764-4912 |
| Exon No. 9: | 4991-5125 |
| Exon No. 10: | 5232-5345 |
| Exon No. 11: | 5494-5604 |
| Exon No. 12: | 5697-5805 |
| Exon No. 13: | 6248-6441 |

Functional Expression of CrtISO in E. coli

E. coli cells of the strain XLI-Blue, carrying plasmids pGB-Ipi and pAC-Zeta accumulate mainly ζ-carotene (Table VI). This plasmid contains the genes CrtE and CrtB, which encode geranylgeranyl pyrophosphate synthase and phytoene synthase, respectively, from Erwinia herbicola, and crtP from Synechococcus PCC7942, which encoded isopentyl diphosphate. When co-transformed with plasmid pT-Zds, which encodes ζ-carotene desaturase from tomato, the cells accumulated mainly prolycopene. A similar result has been previously reported [Bartley, G. E., Scolnik, P. A., and Beyer, P. (1999). Two Arabidopsis thaliana carotene desaturases, phytoene desaturase and ζ-carotene desaturase, expressed in Escherichia coli, catalyze a poly-cis pathway to yield pro-lycopene. Eur. J. Biochem. 259, 396-403]. However, expressing both Zds and CrtISO from plasmid pCrtISO-TZds, resulted in lycopene accumulation (Table VI).

TABLE VI
Functional expression of CrtISO in E. coli

| | | | | | | | | |
|---|---|---|---|---|---|---|---|---|
| | | | | | Di-cis- | | | |
| Genes | Phytoene | Phytofluene | ζ-carotene | Neurosporene | lycopene | Prolycopene | Lycopene | Other |

LIGHT

| | | | | | | | |
|---|---|---|---|---|---|---|---|
| crtE, crtB | 98.4 | | | | | | 1.6 |
| crtE, crtB, Zds, | 99.4 | | | | | | 0.6 |
| CrtISO | | | | | | | |
| crtE, crtB, crtP | 12.2 ± 1.8 | 6.8 ± 0.2 | 79.2 ± 1.5 | | | | 1.8 |
| crtE, crtB, crtP, | 15.2 ± 4.6 | 7.0 ± 0.6 | 75.3 ± 4.7 | | | | 2.5 |
| CrtISO | | | | | | | |
| crtE, crtB, crtP, Zds | 17.9 ± 0.1 | 10.4 ± 0.2 | 54.6 ± 0.6 | 3.6 ± 0.2 | 6.9 ± 0.7 | 6.2 ± 0.5 | 0.4 |
| crtE, crtB, crtP, Zds, | 19.7 ± 3.1 | 11.4 ± 1.2 | 38.5 ± 5.6 | | | 30.1 ± 9.7 | 0.3 |
| CrtISO | | | | | | | |

DARK

| | | | | | | | |
|---|---|---|---|---|---|---|---|
| crtE, crtB | 99.0 | | | | | | 1.0 |
| crtE, crtB, Zds, | 100 | | | | | | |
| CrtISO | | | | | | | |
| crtE, crtB, crtP | 8.9 ± 1.5 | 6.0 ± 0.8 | 83.2 ± 2.2 | | | | 1.9 |
| crtE, crtB, crtP, | 11.7 ± 0.9 | 7.6 ± 0.1 | 78.5 ± 1.3 | | | | 2.2 |
| CrtISO | | | | | | | |
| crtE, crtB, crtP, Zds | 13.7 ± 1.2 | 11.1 ± 1.6 | 66.2 ± 1.9 | | 8.3 ± 1.4 | | 0.7 |
| crtE, crtB, crtP, Zds, | 23.2 ± 1.1 | 16.5 ± 0.9 | 50.0 ± 1.9 | 0.6 ± 0.9 | | 8.5 ± 2.4 | 1.2 |
| CrtISO | | | | | | | |

Cells of E. coli, all carrying plasmid with the gene Ipi, were transfected with different combinations of carotenoid biosynthesis genes. crtE, geranygeranyl diphosphate synthase; crtB, phytoene synthase; crtP, phytoene desaturase; Zds, ζ-carotene desaturase; CrtISO, carotenoid isomerase.
Numbers correspond to percent of total carotenoids.
Others = other carotenoids.

These results clearly indicate that the polypeptide encoded by CrtISO is an authentic carotenoids isomerase which is able to convert in E. coli cis-carotenes to all-trans carotenes.

Expression of CrtISO During Fruit Ripening

To determine the pattern of expression of CrtISO, its mRNA level was measured in different stages of fruit development. In wild type fruits the mRNA levels of CrtISO increased 10 fold during the breaker stage of fruit ripening, similarly to the mode of the expression of the genes Psy and Pds. However, in fruits of tangerine³¹⁸³ the mRNA of CrtISO remained at a very low level throughout fruit ripening. The low levels of expression of CrtISO in tangerine³¹⁸³ is consistent with the mild phenotype compared with the null mutation of tangerine^{mic} phenotype (FIG. 3).

Null Mutation in the Gene sll0033 of Cyanobacterium Synechocystis PCC6803

Null Mutation in the Gene sll0033 of Cyanobacterium Synechocystis

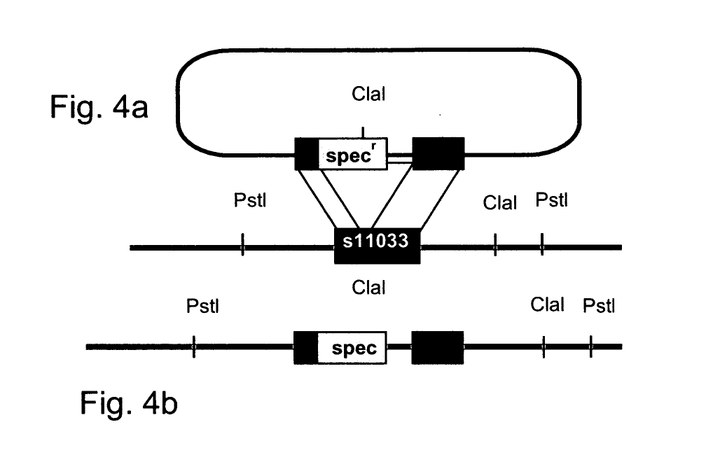

PCC6803 was generated by insertional mutagenesis (FIGS. 4A-B). To this end, two plasmids where constructed.

Plasmid pBS0033 was used to clone the sll0033 gene. The sll0033 sequence was amplified from total genomic DNA of Synechocystis PCC6803 by PCR using the primers 0033F (5′-TTGCTCCGTGTCCGTTGTTAACTT-3′, SEQ ID NO:25) and 0033R (5′-GGCGATCGTGTGAGCTCATTGCTT-3, SEQ ID NO:26) with a high precision reverse transcriptase (PFU Taq polymerase from Stratagene). Primer 0033R contains a single nucleotide mismatch that creates a SacI restriction endonuclease site. The resulting 1611 bp fragment was digested with the SacI restriction endonuclease and cloned into a pBluescript KS(−) plasmid between the sites SacI and EcoRV (blunt) in the polylinker.

Plasmid pBS0033out was used to knock out the endogenous sll0033 gene of the cyanobacterium Synechocystis PCC 6803. A spectinomycin/streptomicyn resistance cassette (M60473) was taken from the pAM1303 plasmid (kindly provided by Dr. Susan Golden; see URL: http://www.bio.tamu.edu/users/sgolden/public/1303.htm) by digestion with the restriction endonucleases BspMKI and CciNI and the ends were filled-in using T4 DNA polymerase. The resulting 2045 bp fragment was inserted in the single filled-in NcoI restriction endonuclease site in the pBS0033 plasmid. This site divides the sll0033 gene in two fragments of 440 bp and 1072 bp. The sll0033 sequences flanking the antibiotic cassette are sufficient to enable efficient homologous recombination with the native cyanobacterial gene. Transfection of the plasmid into the Synechocystis PCC 6803 was performed essentially as described hereinabove. The homologous recombination between the plasmid and the endogenous genome results in the disruption of the endogenous gene and the insertion of the antibiotic-resistance gene in the genome. Selection for stably transformed bacteria was done on spectinomycin selective medium and resistant colonies were isolated. The disruption of the native sll0033 gene as well as the full segregation of the transformed chromosome in these colonies was confirmed by southern blotting of genomic DNA from the mutant. The new strain that was obtained was called Δsll0033.

The carotenoid composition of the wild type (WT) Synechocystis PCC 6803 cyanobacteria and the mutant Δsll0033 Synechocystis grown under light or dark conditions was determined by HPLC. The cultures were grown in liquid BG11 medium under PFD of 30 μE or in complete darkness for 4 days. Carotenoids extracted from the cells were identified by their typical absorbance spectrum and characteristic retention time.

Cells of this mutant accumulated a significant proportion of prolycopene and other cis-carotenoids similarly to the phenotype observed in young or dark-grown green leaves of tangerine^{mic} tomato (Table VII)

TABLE VII
Carotenoid composition in Synechocystis strains

| | | Δsll0033, | | Δsll0033, |
|---|---|---|---|---|
| | WT, light | light | WT, dark | dark |
| ζ-carotene | 0 | 2.1 | 0 | 0.6 |
| Neurosporene | 0 | 1.3 | 0 | 0.1 |
| cis-lycopene | 0 | 3.5 | 0 | 0 |
| Prolycopene | 0 | 0 | 0 | 5.9 |
| Lycopene | 0 | 8.3 | 0 | 5.9 |
| Myxoxanthophyll | 16.1 | 33.1 | 17.1 | 20.4 |
| γ-carotene | 0 | 3.1 | 0 | 1.6 |
| Rubixanthin | 0 | 2.0 | 0 | 0.4 |
| β-carotene | 40.7 | 16.2 | 41.9 | 34.2 |
| β-cryptoxanthin | 2.6 | 1.9 | 2.0 | 1.9 |
| Zeaxanthin | 20.2 | 13.2 | 18.8 | 9.3 |
| Echinenone | 16.6 | 13.6 | 18.2 | 17.9 |
| Hydroxyechinenone | 2.2 | 1.4 | 1.4 | 1.0 |
| Others | 1.6 | 0.3 | 0.6 | 0.9 |
| (+unidentified) | | | | |

Conclusions:

The following conclusions can be derived from the above data:

CRTISO is an authentic carotenoids isomerase, an indispensable function of carotenoid biosynthesis in oxygenic photosynthetic organisms. It is an essential enzyme for producing the all-trans geometric isomers of cis-carotenoids, including phytoene, phytofluene, zeta-carotene, neurosporene and lycopene.

Transgenic expression of CRTISO from tomato in E. coli provides activity of cis to trans isomerization of carotenes, which enhances carotenoid biosynthesis and hence increases their concentration in the cells.

CRTISO is conserved among photosynthetic organisms where phytoene conversion to lycopene through four dehydrogenation steps is carried out by the enzymes PDS and ZDS. In Arabidopsis, a gene annotated as Pdh (GeneBank accession No. AC011001, SEQ ID NO:22) encodes a polypeptide (SEQ ID NO:23) that is 75% identical to CRTISO from tomato, and in the cyanobacterium Synechocystis PCC6803 the polypeptide (SEQ ID NO:20) encoded by sll0033 (http://www.kazusa.or.jp/cyano/, SEQ ID NO:19) is 60% identical to the mature CRTISO polypeptide. A null mutation in the gene sll0033 of cyanobacterium Synechocystis PCC6803 was generated by insertion mutagenesis. Cells of this mutant accumulated a significant proportion of prolycopene and other cis-carotenoids similarly to the phenotype observed in young or dark-grown green leaves of tangerine^{mic} tomato, demonstrating is has isomerase activity.

In tangerine tomato mutants, CrtISO is mutated: (a) A deletion mutation in CrtISO, which nullifies its function, was discovered in the allele tangerine^{mic} that exhibits a typical tangerine phenotype; and (b) abolition of expression in fruits of CrtISO was detected in tangerine³¹⁸³.

A dinucleotide-binding motif in the amino terminus of the CRTISO polypeptide is characteristic of all carotenoid desaturases identified up to now, and is also present in various lycopene cyclases. Its existence suggests that the carotene isomerase, possibly flavo-protein, is engaged in a redox related reaction in which a temporary abstraction of electrons takes place.

The function of carotene isomerase in plants is to enable carotenoid biosynthesis in the dark and in non-photosynthetic tissues. This is essential in germinating seedlings, in roots and in chromoplasts in the absence of chlorophyll sensitization.

CrtISO from tomato is expressed in all green tissues but is up-regulated during fruit ripening and in flowers.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. An isolated nucleic acid comprising a polynucleotide encoding a polypeptide having an amino acid sequence at least 75% similar to SEQ ID NO:15, as determined using the Standard protein-protein BLAST [blastp] software of the NCBI, said polypeptide having carotenoids isomerase catalytic activity.

2. The isolated nucleic acid of claim 1, wherein said polynucleotide comprises a cDNA.

3. The isolated nucleic acid of claim 1, wherein said polynucleotide comprises a genomic DNA.

4. The isolated nucleic acid of claim 1, wherein said polynucleotide comprises at least one intron sequence.

5. The isolated nucleic acid of claim 1, wherein said polynucleotide is intronless.

6. The isolated nucleic acid of claim 1, wherein said polypeptide has an amino acid sequence at least 80% similar to SEQ ID NO:15, as determined using the Standard protein-protein BLAST [blastp] software of the NCBI.

7. The isolated nucleic acid of claim 1, wherein said polypeptide has an amino acid sequence at least 85% similar to SEQ ID NO:15, as determined using the Standard protein-protein BLAST [blastp] software of the NCBI.

8. The isolated nucleic acid of claim 1, wherein said polypeptide has an amino acid sequence at least 90% similar to SEQ ID NO:15, as determined using the Standard protein-protein BLAST [blastp] software of the NCBI.

9. The isolated nucleic acid of claim 1, wherein said polypeptide has an amino acid sequence at least 95% similar to SEQ ID NO:15, as determined using the Standard protein-protein BLAST [blastp] software of the NCBI.

10. The isolated nucleic acid of claim 1, wherein said polypeptide comprises an amino acid sequence as set forth in SEQ ID NO:15.

11. The isolated nucleic acid of claim 1, wherein said polynucleotide comprises a nucleotide sequence as set forth between positions 421-2265 of SEQ ID NO:14.

12. The isolated nucleic acid of claim 1, wherein said polynucleotide comprises a nucleotide sequence as set forth at positions 1341-6442 of SEQ ID NO:16.

13. The isolated nucleic acid of claim 1, further comprising a promoter operably linked to said polynucleotide in a sense orientation, so as to produce a RNA encoding said polypeptide.

14. The isolated nucleic acid of claim 1, further comprising a promoter operably linked to said polynucleotide in an antisense orientation, so as to produce a RNA hybridizeable with a RNA encoding said polypeptide.

15. A vector comprising the isolated nucleic acid of claim 13.

16. A vector comprising the isolated nucleic acid of claim 14.

17. A vector comprising the isolated nucleic acid of claim 1.

18. The vector of claim 17, wherein said vector is suitable for expression in a eukaryote.

19. The vector of claim 17, wherein said vector is suitable for expression in a prokaryote.

20. The vector of claim 17, wherein said vector is suitable for expression in a plant.

21. A transduced organism genetically transduced by the nucleic acid of claim 1.

22. The transduced organism of claim 21, wherein the organism is a eukaryote.

23. The transduced organism of claim 21, wherein the organism is a prokaryote.

24. The transduced organism of claim 21, wherein the organism is a plant.

25. An isolated nucleic acid comprising a polynucleotide at least 75% identical to positions 421-2265 of SEQ ID NO:14 or to positions 1341-6442 of SEQ ID NO:16, as determined using the Standard nucleotide-nucleotide BLAST [blastn] software of the NCBI.

26. The isolated nucleic acid of claim 25, wherein said polynucleotide comprises a cDNA.

27. The isolated nucleic acid of claim 25, wherein said polynucleotide comprises a genomic DNA.

28. The isolated nucleic acid of claim 25, wherein said polynucleotide comprises at least one intron sequence.

29. The isolated nucleic acid of claim 25, wherein said polynucleotide is intronless.

30. The isolated nucleic acid of claim 25, wherein said polynucleotide is at least 80% identical to positions 421-2265 of SEQ ID NO:14 or to positions 1341-6442 of SEQ ID NO:16, as determined using the Standard nucleotide-nucleotide BLAST [blastn] software of the NCBI.

31. The isolated nucleic acid of claim 25, wherein said polynucleotide is at least 85% identical to positions 421-2265 of SEQ ID NO:14 or to positions 1341-6442 of SEQ ID NO:16, as determined using the Standard nucleotide-nucleotide BLAST [blastn] software of the NCBI.

32. The isolated nucleic acid of claim 25, wherein said polynucleotide is at least 90% identical to positions 421-2265 of SEQ ID NO:14 or to positions 1341-6442 of SEQ ID NO:16, as determined using the Standard nucleotide-nucleotide BLAST [blastn] software of the NCBI.

33. The isolated nucleic acid of claim 25, wherein said polynucleotide is at least 95% identical to positions 421-2265 of SEQ ID NO:14 or to positions 1341-6442 of SEQ ID NO:16, as determined using the Standard nucleotide-nucleotide BLAST [blastn] software of the NCBI.

34. The isolated nucleic acid of claim 25, wherein said polynucleotide is identical to positions 421-2265 of SEQ ID NO:14 or to positions 1341-6442 of SEQ ID NO:16, as determined using the Standard nucleotide-nucleotide BLAST [blastn] software of the NCBI.

35. The isolated nucleic acid of claim 25, wherein said polynucleotide comprises a nucleotide sequence as set forth between positions 421-2265 of SEQ ID NO:14.

36. The isolated nucleic acid of claim 25, wherein said polynucleotide comprises a nucleotide sequence as set forth at positions 1341-6442 of SEQ ID NO:16.

37. The isolated nucleic acid of claim 25, further comprising a promoter operably linked to said polynucleotide in a sense orientation.

38. The isolated nucleic acid of claim 25, further comprising a promoter operably linked to said polynucleotide in an antisense orientation.

39. A vector comprising the isolated nucleic acid of claim 37.

40. A vector comprising the isolated nucleic acid of claim 38.

41. A vector comprising the isolated nucleic acid of claim 25.

42. The vector of claim 41, wherein said vector is suitable for expression in a eukaryote.

43. The vector of claim 41, wherein said vector is suitable for expression in a prokaryote.

44. The vector of claim 41, wherein said vector is suitable for expression in a plant.

45. A transduced organism genetically transduced by the nucleic acid of claim 25.

46. The transduced organism of claim 45, wherein the organism is a eukaryote.

47. The transduced organism of claim 45, wherein the organism is a prokaryote.

48. The transduced organism of claim 45, wherein the organism is a plant.

49. A transduced cell expressing from a transgene a recombinant polypeptide having an amino acid sequence at least 50% similar to SEQ ID NO:15, as determined using the Standard protein-protein BLAST [blastp] software of the NCBI, said polypeptide having a carotenoids isomerase catalytic activity, the cell having a level of said carotenoids isomerase catalytic activity over that of a non-transduced and otherwise similar cell.

50. The transduced cell of claim 49, wherein said polypeptide is at least 55% similar to SEQ ID NO:15, as determined using the Standard protein-protein BLAST [blastp] software of the NCBI.

51. The transduced cell of claim 49, wherein said polypeptide is at least 60% similar to SEQ ID NO:15, as determined using the Standard protein-protein BLAST [blastp] software of the NCBI.

52. The transduced cell of claim 49, wherein said polypeptide is at least 65% similar to SEQ ID NO:15, as determined using the Standard protein-protein BLAST [blastp] software of the NCBI.

53. The transduced cell of claim 49, wherein said polypeptide is at least 70% similar to SEQ ID NO:15, as determined using the Standard protein-protein BLAST [blastp] software of the NCBI.

54. The transduced cell of claim 49, wherein said polypeptide is at least 75% similar to SEQ ID NO:15, as determined using the Standard protein-protein BLAST [blastp] software of the NCBI.

55. The transduced cell of claim 49, wherein said polypeptide is at least 80% similar to SEQ ID NO:15, as determined using the Standard protein-protein BLAST [blastp] software of the NCBI.

56. The transduced cell of claim 49, wherein said polypeptide is at least 85% similar to SEQ ID NO:15, as determined using the Standard protein-protein BLAST [blastp] software of the NCBI.

57. The transduced cell of claim 49, wherein said polypeptide is at least 90% similar to SEQ ID NO:15, as determined using the Standard protein-protein BLAST [blastp] software of the NCBI.

58. The transduced cell of claim 49, wherein said polypeptide is at least 95% similar to SEQ ID NO:15, as determined using the Standard protein-protein BLAST [blastp] software of the NCBI.

59. The transduced cell of claim 49, wherein said polypeptide comprises an amino acids sequence as set forth in SEQ ID NO:15.

60. The transduced cell of claim 49, wherein the cell is a eukaryotic cell.

61. The transduced cell of claim 49, wherein the cell is a prokaryotic cell.

62. The transduced cell of claim 49, wherein the cell is a plant cell.

63. The transduced cell of claim 49, wherein the cell forms a part of a transgenic plant.

64. A transgenic plant having cells expressing from a transgene a recombinant polypeptide having an amino acid sequence at least 50% similar to SEQ ID NO:15, as determined using the Standard protein-protein BLAST [blastp] software of the NCBI, said polypeptide having a carotenoids isomerase catalytic activity, the cell having a level of said carotenoids isomerase catalytic activity over that of a non-transduced and otherwise similar cell.

65. The transgenic plant of claim 64, wherein said polypeptide is at least 55% similar to SEQ ID NO:15, as determined using the Standard protein-protein BLAST [blastp] software of the NCBI.

66. The transgenic plant of claim 64, wherein said polypeptide is at least 60% similar to SEQ ID NO:15, as determined using the Standard protein-protein BLAST [blastp] software of the NCBI.

67. The transgenic plant of claim 64, wherein said polypeptide is at least 65% similar to SEQ ID NO:15, as determined using the Standard protein-protein BLAST [blastp] software of the NCBI.

68. The transgenic plant of claim 64, wherein said polypeptide is at least 70% similar to SEQ ID NO:15, as determined using the Standard protein-protein BLAST [blastp] software of the NCBI.

69. The transgenic plant of claim 64, wherein said polypeptide is at least 75% similar to SEQ ID NO:15, as determined using the Standard protein-protein BLAST [blastp] software of the NCBI.

70. The transgenic plant of claim 64, wherein said polypeptide is at least 80% similar to SEQ ID NO:15, as determined using the Standard protein-protein BLAST [blastp] software of the NCBI.

71. The transgenic plant of claim 64, wherein said polypeptide is at least 85% similar to SEQ ID NO:15, as determined using the Standard protein-protein BLAST [blastp] software of the NCBI.

72. The transgenic plant of claim 64, wherein said polypeptide is at least 90% similar to SEQ ID NO:15, as determined using the Standard protein-protein BLAST [blastp] software of the NCBI.

73. The transgenic plant of claim 64, wherein said polypeptide is at least 95% similar to SEQ ID NO:15, as determined using the Standard protein-protein BLAST [blastp] software of the NCBI.

74. The transgenic plant of claim 64, wherein said polypeptide comprises an amino acids sequence as set forth in SEQ ID NO:15.

75. A method of increasing a content of all-trans geometric isomers of carotenoids in a carotenoids producing cell, the method comprising, expressing in said cell, from a transgene, a recombinant polypeptide having an amino acid sequence at least 50% similar to SEQ ID NO:15, as determined using the Standard protein-protein BLAST [blastp] software of the NCBI, said polypeptide having a carotenoids isomerase catalytic activity.

76. The method of claim 75, wherein said polypeptide is at least 55% similar to SEQ ID NO:15, as determined using the Standard protein-protein BLAST [blastp] software of the NCBI.

77. The method of claim 75, wherein said polypeptide is at least 60% similar to SEQ ID NO:15, as determined using the Standard protein-protein BLAST [blastp] software of the NCBI.

78. The method of claim 75, wherein said polypeptide is at least 65% similar to SEQ ID NO:15, as determined using the Standard protein-protein BLAST [blastp] software of the NCBI.

79. The method of claim 75, wherein said polypeptide is at least 70% similar to SEQ ID NO:15, as determined using the Standard protein-protein BLAST [blastp] software of the NCBI.

80. The method of claim 75, wherein said polypeptide is at least 75% similar to SEQ ID NO:15, as determined using the Standard protein-protein BLAST [blastp] software of the NCBI.

81. The method of claim 75, wherein said polypeptide is at least 80% similar to SEQ ID NO:15, as determined using the Standard protein-protein BLAST [blastp] software of the NCBI.

82. The method of claim 75, wherein said polypeptide is at least 85% similar to SEQ ID NO:15, as determined using the Standard protein-protein BLAST [blastp] software of the NCBI.

83. The method of claim 75, wherein said polypeptide is at least 90% similar to SEQ ID NO:15, as determined using the Standard protein-protein BLAST [blastp] software of the NCBI.

84. The method of claim 75, wherein said polypeptide is at least 95% similar to SEQ ID NO:15, as determined using the Standard protein-protein BLAST [blastp] software of the NCBI.

85. The method of claim 75, wherein said polypeptide comprises an amino acids sequence as set forth in SEQ ID NO:15.

86. A method of decreasing a content of all-trans geometric isomers of carotenoids in a carotenoids producing cell, the method comprising, expressing in said cell, from a transgene, a RNA molecule capable of reducing a level of a natural RNA encoding a carotenoids isomerase in said cell.

87. The method of claim 86, wherein said RNA molecule is antisense RNA, operative via antisense inhibition.

88. The method of claim 86, wherein said RNA molecule is sense RNA, operative via RNA inhibition.

89. The method of claim 86, wherein said RNA molecule is a ribozyme, operative via ribozyme cleavage inhibition.

90. The method of claim 86, wherein said RNA molecule comprises a sequence at least 50% complementary to a stretch of at least 15 contiguous nucleotides between positions 421-2265 of SEQ ID NO:14, as determined using the Standard nucleotide-nucleotide BLAST [blastn] software of the NCBI.

91. The method of claim 86, wherein said RNA molecule comprises a sequence at least 55% complementary to a stretch of at least 15 contiguous nucleotides between positions 421-2265 of SEQ ID NO:14, as determined using the Standard nucleotide-nucleotide BLAST [blastn] software of the NCBI.

92. The method of claim 86, wherein said RNA molecule comprises a sequence at least 60% complementary to a stretch of at least 15 contiguous nucleotides between positions 421-2265 of SEQ ID NO:14, as determined using the Standard nucleotide-nucleotide BLAST [blastn] software of the NCBI.

93. The method of claim 86, wherein said RNA molecule comprises a sequence at least 65% complementary to a stretch of at least 15 contiguous nucleotides between positions 421-2265 of SEQ ID NO:14, as determined using the Standard nucleotide-nucleotide BLAST [blastn] software of the NCBI.

94. The method of claim 86, wherein said RNA molecule comprises a sequence at least 70% complementary to a stretch of at least 15 contiguous nucleotides between positions 421-2265 of SEQ ID NO:14, as determined using the Standard nucleotide-nucleotide BLAST [blastn] software of the NCBI.

95. The method of claim 86, wherein said RNA molecule comprises a sequence at least 75% complementary to a stretch of at least 15 contiguous nucleotides between positions 421-2265 of SEQ ID NO:14, as determined using the Standard nucleotide-nucleotide BLAST [blastn] software of the NCBI.

96. The method of claim 86, wherein said RNA molecule comprises a sequence at least 80% complementary to a stretch of at least 15 contiguous nucleotides between positions 421-2265 of SEQ ID NO:14, as determined using the Standard nucleotide-nucleotide BLAST [blastn] software of the NCBI.

97. The method of claim 86, wherein said RNA molecule comprises a sequence at least 85% complementary to a stretch of at least 15 contiguous nucleotides between positions 421-2265 of SEQ ID NO:14, as determined using the Standard nucleotide-nucleotide BLAST [blastn] software of the NCBI.

98. The method of claim 86, wherein said RNA molecule comprises a sequence at least 90% complementary to a stretch of at least 15 contiguous nucleotides between positions 421-2265 of SEQ ID NO:14, as determined using the Standard nucleotide-nucleotide BLAST [blastn] software of the NCBI.

99. The method of claim 86, wherein said RNA molecule comprises a sequence at least 95% complementary to a stretch of at least 15 contiguous nucleotides between positions 421-2265 of SEQ ID NO:14, as determined using the Standard nucleotide-nucleotide BLAST [blastn] software of the NCBI.

100. The method of claim 86, wherein said RNA is complementary to a stretch of at least 15 contiguous nucleotides between positions 421-2265 of SEQ ID NO:14.

101. A method of modulating a ratio between all-trans geometric isomers of carotenoids and cis-carotenoids in a carotenoids producing cell, the method comprising, expressing in said cell, from a transgene, a RNA molecule capable of modulating a level of RNA encoding a carotenoids isomerase in said cell.

102. The method of claim 101, wherein said RNA molecule is antisense RNA, operative via antisense inhibition, thereby decreasing said ratio.

103. The method of claim 101, wherein said RNA molecule is sense RNA, operative via RNA inhibition, thereby decreasing said ratio.

104. The method of claim 101, wherein said RNA molecule is a ribozyme, operative via ribozyme cleavage inhibition, thereby decreasing said ratio.

105. The method of claim 101, wherein said RNA molecule is sense RNA augmenting a level of said RNA encoding said carotenoids isomerase, thereby increasing said ratio.

106. The method of claim 101, wherein said RNA molecule comprises a sequence at least 50% identical to positions 421-2265 of SEQ ID NO:14, as determined using the Standard nucleotide-nucleotide BLAST [blastn] software of the NCBI, and encoding a polypeptide having a carotenoids isomerase catalytic activity.

107. The method of claim 101, wherein said RNA molecule comprises a sequence at least 50% complementary to a stretch of at least 15 contiguous nucleotides between positions 421-2265 of SEQ ID NO:14, as determined using the Standard nucleotide-nucleotide BLAST [blastn] software of the NCBI.

108. The method of claim 101, wherein said RNA molecule comprises a sequence at least 55% complementary to a stretch of at least 15 contiguous nucleotides between positions 421-2265 of SEQ ID NO:14, as determined using the Standard nucleotide-nucleotide BLAST [blastn] software of the NCBI.

109. The method of claim 101, wherein said RNA molecule comprises a sequence at least 60% complementary to a stretch of at least 15 contiguous nucleotides between positions 421-2265 of SEQ ID NO:14, as determined using the Standard nucleotide-nucleotide BLAST [blastn] software of the NCBI.

110. The method of claim 101, wherein said RNA molecule comprises a sequence at least 65% complementary to a stretch of at least 15 contiguous nucleotides between positions 421-2265 of SEQ ID NO:14, as determined using the Standard nucleotide-nucleotide BLAST [blastn] software of the NCBI.

111. The method of claim 101, wherein said RNA molecule comprises a sequence at least 70% complementary to a stretch of at least 15 contiguous nucleotides between positions 421-2265 of SEQ ID NO:14, as determined using the Standard nucleotide-nucleotide BLAST [blastn] software of the NCBI.

112. The method of claim 101, wherein said RNA molecule comprises a sequence at least 75% complementary to a stretch of at least 15 contiguous nucleotides between positions 421-2265 of SEQ ID NO:14, as determined using the Standard nucleotide-nucleotide BLAST [blastn] software of the NCBI.

113. The method of claim 101, wherein said RNA molecule comprises a sequence at least 80% complementary to a stretch of at least 15 contiguous nucleotides between positions 421-2265 of SEQ ID NO:14, as determined using the Standard nucleotide-nucleotide BLAST [blastn] software of the NCBI.

114. The method of claim 101, wherein said RNA molecule comprises a sequence at least 85% complementary to a stretch of at least 15 contiguous nucleotides between positions 421-2265 of SEQ ID NO:14, as determined using the Standard nucleotide-nucleotide BLAST [blastn] software of the NCBI.

115. The method of claim 101, wherein said RNA molecule comprises a sequence at least 90% complementary to a stretch of at least 15 contiguous nucleotides between positions 421-2265 of SEQ ID NO:14, as determined using the Standard nucleotide-nucleotide BLAST [blastn] software of the NCBI.

116. The method of claim 101, wherein said RNA molecule comprises a sequence at least 95% complementary to a stretch of at least 15 contiguous nucleotides between positions 421-2265 of SEQ ID NO:14, as determined using the Standard nucleotide-nucleotide BLAST [blastn] software of the NCBI.

117. The method of claim 101, wherein said RNA molecule comprises a sequence as set forth in SEQ ID NO:14.

118. A method of decreasing a content of all-trans geometric isomers of carotenoids in a carotenoids producing cell, the method comprising, introducing into the cell an antisense nucleic acid molecule capable of reducing a level of a natural mRNA encoding a carotenoids isomerase in said cell via at least one antisense mechanism.

119. The method of claim 118, wherein said antisense nucleic acid molecule is antisense RNA.

120. The method of claim 118, wherein said antisense nucleic acid molecule is an antisense oligonucleotide of at least 15 nucleotides.

121. The method of claim 118, wherein said antisense nucleic acid molecule comprises a sequence at least 50% complementary to a stretch of at least 15 contiguous nucleotides between positions 421-2265 of SEQ ID NO:14, as determined using the Standard nucleotide-nucleotide BLAST [blastn] software of the NCBI.

122. The method of claim 118, wherein said antisense nucleic acid molecule comprises a sequence at least 55% complementary to a stretch of at least 15 contiguous nucleotides between positions 421-2265 of SEQ ID NO:14, as determined using the Standard nucleotide-nucleotide BLAST [blastn] software of the NCBI.

123. The method of claim 118, wherein said antisense nucleic acid molecule comprises a sequence at least 60% complementary to a stretch of at least 15 contiguous nucleotides between positions 421-2265 of SEQ ID NO:14, as determined using the Standard nucleotide-nucleotide BLAST [blastn] software of the NCBI.

124. The method of claim 118, wherein said antisense nucleic acid molecule comprises a sequence at least 65% complementary to a stretch of at least 15 contiguous nucleotides between positions 421-2265 of SEQ ID NO:14, as determined using the Standard nucleotide-nucleotide BLAST [blastn] software of the NCBI.

125. The method of claim 118, wherein said antisense nucleic acid molecule comprises a sequence at least 70% complementary to a stretch of at least 15 contiguous nucleotides between positions 421-2265 of SEQ ID NO:14, as determined using the Standard nucleotide-nucleotide BLAST [blastn] software of the NCBI.

126. The method of claim 118, wherein said antisense nucleic acid molecule comprises a sequence at least 75% complementary to a stretch of at least 15 contiguous nucleotides between positions 421-2265 of SEQ ID NO:14, as determined using the Standard nucleotide-nucleotide BLAST [blastn] software of the NCBI.

127. The method of claim 118, wherein said antisense nucleic acid molecule comprises a sequence at least 80% complementary to a stretch of at least 15 contiguous nucleotides between positions 421-2265 of SEQ ID NO:14, as determined using the Standard nucleotide-nucleotide BLAST [blastn] software of the NCBI.

128. The method of claim 118, wherein said antisense nucleic acid molecule comprises a sequence at least 85% complementary to a stretch of at least 15 contiguous nucleotides between positions 421-2265 of SEQ ID NO:14, as determined using the Standard nucleotide-nucleotide BLAST [blastn] software of the NCBI.

129. The method of claim 118, wherein said antisense nucleic acid molecule comprises a sequence at least 90% complementary to a stretch of at least 15 contiguous nucleotides between positions 421-2265 of SEQ ID NO:14, as determined using the Standard nucleotide-nucleotide BLAST [blastn] software of the NCBI.

130. The method of claim 118, wherein said antisense nucleic acid molecule comprises a sequence at least 95% complementary to a stretch of at least 15 contiguous nucleotides between positions 421-2265 of SEQ ID NO:14, as determined using the Standard nucleotide-nucleotide BLAST [blastn] software of the NCBI.

131. The method of claim 118, wherein said antisense nucleic acid molecule is complementary to a stretch of at least 15 contiguous nucleotides between positions 421-2265 of SEQ ID NO:14, as determined using the Standard nucleotide-nucleotide BLAST [blastn] software of the NCBI.

132. The method of claim 120, wherein said oligonucleotide is a synthetic oligonucleotide and comprises a man-made modification rendering said synthetic oligonucleotide more stable in cell environment.

133. The method of claim 132, wherein said synthetic oligonucleotide is selected from the group consisting of methylphosphonate oligonucleotide, monothiophosphate oligonucleotide, dithiophosphate oligonucleotide, phosphoramidate oligonucleotide, phosphate ester oligonucleotide, bridged phosphorothioate oligonucleotide, bridged phosphoramidate oligonucleotide, bridged methylenephosphonate oligonucleotide, dephospho internucleotide analogs with siloxane bridges, carbonate bridge oligonucleotide, carboxymethyl ester bridge oligonucleotide, carbonate bridge oligonucleotide, carboxymethyl ester bridge oligonucleotide, acetamide bridge oligonucleotide, carbamate bridge oligonucleotide, thioether bridge oligonucleotide, sulfoxy bridge oligonucleotide, sulfono bridge oligonucleotide and α-anomeric bridge oligonucleotide.

134. An expression construct for directing an expression of a gene-of-interest in a plant tissue, the expression construct comprising a regulatory sequence of CrtISO of tomato.

135. The expression construct of claim 134, wherein said plant tissue is selected from the group consisting of flower, fruit and leaves.

136. A method of isolating a polynucleotide encoding a polypeptide having an amino acid sequence at least 50% similar to SEQ ID NO:15 and hence potentially having a carotenoids isomerase catalytic activity from a carotenoid producing species, the method comprising screening a cDNA or genomic DNA library prepared from isolated RNA or genomic DNA extracted from said species with a nucleic acid probe of at least 15 nucleotides and being at least 50% identical to a contiguous stretch of nucleotides of SEQ ID NO:14 or 16 or their complementary sequences and isolating clones reacting with said probe.

137. A method of isolating a polynucleotide encoding a polypeptide having an amino acid sequence at least 50% similar to SEQ ID NO:15 and hence potentially having a carotenoids isomerase catalytic activity from a carotenoid producing species, the method comprising providing at least one PCR primer of at least 15 nucleotides being at least 50% identical to a contiguous stretch of nucleotides of SEQ ID NO:14 or 16 or their complementary sequences and using said at least one PCR primer in a PCR reaction to amplify at least a segment of said polynucleotide from DNA or cDNA derived from said species.

138. An isolated polypeptide comprising an amino acid sequence at least 75% similar to SEQ ID NO:15, as determined using the Standard protein-protein BLAST [blastp] software of the NCBI, said polypeptide having carotenoids isomerase catalytic activity.

139. The isolated polypeptide of claim 138, wherein said amino acid sequence is at least 80% similar to SEQ ID NO:15, as determined using the Standard protein-protein BLAST [blastp] software of the NCBI.

140. The isolated polypeptide of claim 138, wherein said amino acid sequence is at least 85% similar to SEQ ID NO:15, as determined using the Standard protein-protein BLAST [blastp] software of the NCBI.

141. The isolated polypeptide of claim 138, wherein said amino acid sequence is at least 90% similar to SEQ ID NO:15, as determined using the Standard protein-protein BLAST [blastp] software of the NCBI.

142. The polypeptide acid of claim 138, wherein said amino acid sequence is at least 95% similar to SEQ ID NO:15, as determined using the Standard protein-protein BLAST [blastp] software of the NCBI.

143. The polypeptide acid of claim 138, wherein said amino acid sequence is as set forth in SEQ ID NO:15.
